(19) United States
(12) Patent Application Publication
(10) Pub. No.: US 2023/0302120 A1
(43) Pub. Date: Sep. 28, 2023

(54) PROTEINS, POLYNUCLEOTIDES, AND METHODS FOR TREATING CORONAVIRUS INFECTION

(71) Applicants: THE BOARD OF REGENTS OF THE UNIVERSITY OF TEXAS SYSTEM, Austin, TX (US); WESTPORT BIO, LLC, Sugar Land, TX (US)

(72) Inventors: Ashok K. Chopra, Galveston, TX (US); Jian Sha, Galveston, TX (US); Eric Rothe, Sugar Land, TX (US); Snehal Patel, Sugar Land, TX (US)

(21) Appl. No.: 18/020,659

(22) PCT Filed: Aug. 11, 2021
(86) PCT No.: PCT/US2021/045547

§ 371 Date: Feb. 10, 2023

Related U.S. Application Data
(60) Provisional application No. 63/064,083, filed on Aug. 11, 2020.

(51) Int. Cl. A61K 39/215; A61P 37/04

(52) U.S. Cl. CPC A61K 39/215; A61P 37/04; A61K 2039/575

(57) ABSTRACT

Provided herein are proteins that include different combinations of structural and nonstructural proteins from SARS-CoV-2 and MERS-CoV. Also provided are polynucleotides encoding the proteins of the present disclosure. In one embodiment, a polynucleotide encoding a protein is present as a viral vector, such as adenovirus. In one embodiment, a polynucleotide encoding a protein is present as a mRNA. Also provided are methods for using the proteins and polynucleotides of the present disclosure.

### CROSS-REFERENCE TO RELATED APPLICATIONS

This application claims priority to U.S. Provisional Patent Application Ser. No. 63/064,083, filed Aug. 11, 2020, the disclosure of which is incorporated herein by reference.

### SEQUENCE LISTING

This application contains a Sequence Listing electronically submitted via EFS-Web to the United States Patent and Trademark Office as an ASCII text file entitled “0265-000094WO01-Seq-Listing_ST25.txt” having a size of 201 kilobytes and created on Aug. 10, 2021. The information contained in the Sequence Listing is incorporated by reference herein.

### BACKGROUND

Coronaviruses (CoVs) are notorious in crossing animal-to-animal and animal-to-human species barriers, with some having been emerged as significant pathogens posing unprecedented threats to public health, and grossly affecting world's economy and the healthcare systems (Chan et al., Emerg Microbes Infect 9, 221-236 (2020); Shang et al., NPJ Vaccines 5, 18 (2020)). Prior to December 2019, six CoVs were known to infect humans and included: HCoV-229E, HKU-NL63, HCoV-OC43, HCoV-HKU1, Severe Acute Respiratory Syndrome (SARS)-CoV and MERS (Middle East Respiratory Syndrome)-CoV. The first four CoVs, HCoV-229E, HKU-NL63, HCoV-OC43 and HCoV-HKU1, generally lead to self-limiting upper respiratory tract infections in immunocompetent hosts, and occasionally, lower respiratory tract infections in immunocompromised individuals and elderly (Chan et al., Emerg Microbes Infect 9, 221-236 (2020)). In contrast, SARS-CoV and MERS-CoV emerged in 2003 and 2012, respectively, and were the cause of severe lower respiratory tract infections which were associated with acute respiratory distress syndrome and extrapulmonary manifestations, and a mortality rate of ˜10-36% (Chan et al., Emerg Microbes Infect 9, 221-236 (2020); Peiris et al., Lancet 361, 1319-1325 (2003); Yeung et al., Nat Microbiol 1, 16004 (2016)). The ongoing pandemic linked to a novel highly transmissible coronavirus, designated as SARS-CoV-2 or nCoV, which originated in China (mid-December 2019), and an etiological agent of atypical pneumonia, has now spread to almost all the nations in the world with more than 196 million confirmed cases and over 4 million deaths, with the numbers still rising (available on the world wide web atcoronavirus.jhu.edu/map.html).

### SUMMARY OF THE APPLICATION

The present disclosure is directed to proteins that include proteins encoded by a coronavirus, such as SARS-CoV-2 and/or MERS-CoV. Also provided are polynucleotide sequences encoding the proteins and antibody that binds a protein. A polynucleotide can be present in a vector, such as a plasmid vector or a viral vector. Also provided herein are compositions that include one or more proteins, one or more polynucleotides, and/or one or more antibody. Further provided by the present disclosure are methods of using the proteins, polynucleotides, antibody, and compositions that include proteins, polynucleotides, and/or antibody. The methods include inducing an immune response, treating an infection, treating a sign of infection, and/or treating a condition. In some embodiments, methods that include administrations of alternating doses, simultaneous doses, combinations of compositions disclosed herein, and/or administrations preceding or following administrations of currently available vaccines increase the efficacy and safety beyond that of current treatments.

Terms used herein will be understood to take on their ordinary meaning in the relevant art unless specified otherwise. Several terms used herein, and their meanings are set forth below.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, fusion protein, and polypeptide are all included within the definition of protein and these terms are used interchangeably.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. In one embodiment, a polynucleotide is isolated. A polynucleotide can be linear or circular in topology. A polynucleotide can be, for example, a portion of a vector, such as an expression or cloning vector, or a fragment.

As used herein, an “isolated” substance is one that has been removed from a cell and many of the proteins, nucleic acids, and other cellular material of its natural environment, or the environment in which it was expressed, are no longer present. A substance may be purified, i.e., at least 60% free, at least 75% free, or at least 90% free from other components with which they are naturally associated. Proteins and polynucleotides that are produced by recombinant, enzymatic, or chemical techniques are considered to be isolated and purified by definition, since they were never present in a cell. For instance, a protein, a polynucleotide, or a viral particle can be isolated or purified.

As used herein, the terms “coding region,” “coding sequence,” and “open reading frame” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

While the polynucleotide sequences described herein are listed as DNA sequences, it is understood that the complements, reverse sequences, and reverse complements of the DNA sequences can be easily determined by the skilled person. It is also understood that the sequences disclosed herein as DNA sequences can be converted from a DNA sequence to a RNA sequence by replacing each thymidine nucleotide with a uridine nucleotide. For instance, in embodiments where a polynucleotide described herein is a mRNA that can be used as a vaccine, the polynucleotide at SEQ ID NO:12, 14, 16, 18, 20, 22, 24, 26, 28, 30, or 32 can be converted from a DNA sequence to a RNA sequence by replacing each thymidine nucleotide with a uridine nucleotide.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

The terms “comprises,” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided. The term “consisting of” means including, and limited to, whatever follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

Unless otherwise specified, “a,” “an,” “the,” and “at least one” are used interchangeably and mean one or more than one.

Conditions that are “suitable” for an event to occur, or “suitable” conditions, are conditions that do not prevent such events from occurring. Thus, these conditions permit, enhance, facilitate, and/or are conducive to the event.

As used herein, “providing” in the context of a composition, an article, or a nucleic acid, means making the composition, article, or nucleic acid, purchasing the composition, article, or nucleic acid, or otherwise obtaining the compound, composition, article, or nucleics acid.

Also, herein, the recitations of numerical ranges by endpoints include all numbers subsumed within that range (e.g., 1 to 5 includes 1, 1.5, 2, 2.75, 3, 3.80, 4, 5, etc.).

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible Subranges as well as individual numerical values within that range. For example, description of a range such as from 1 to 6 should be considered to have specifically disclosed Subranges such as from 1 to 3, from 1 to 4, from 1 to 5, from 2 to 4, from 2 to 6, from 3 to 6 etc., as well as individual numbers within that range, for example, 1, 2, 2.7.3, 4, 5, 5.3, and 6. This applies regardless of the breadth of the range.

### BRIEF DESCRIPTION OF THE FIGURES

The following detailed description of illustrative embodiments of the present disclosure may be best understood when read in conjunction with the following drawings.

FIG. 1 shows examples of amino acid sequences of domains that can be present in proteins described herein.

FIG. 2 shows examples of amino acid sequences of proteins described herein and examples of nucleotide sequences encoding the proteins.

FIG. 3 shows expression of gfp gene encoded by vaccine constructs.

FIG. 4 shows expression of transgenes in A549 cells. A549 cells were grown in MEM with 10% FBS at 37° C.+5% CO₂ to 80% confluency. The cells were infected with 1000 virus particles of construct #s 1 and 6. After 72 h of infection, the host cells were harvested with RIPA buffer and briefly sonicated to shear DNA. Cell lysates were diluted 1:10 in PBS, and 10 μL was added to 10 μL of 4×SDS-PAGE loading buffer with β-mercaptoethanol. Samples were boiled for 5 min, loaded to a 4-20% Mini-PROTEAN Bio-Rad gel, and run for 1 h at 150V before transferring to the PVDF membrane. After transfer, the membrane was blocked with 5% skim milk powder-PBS (pH 7.4) at room temperature (RT) for 1 h with gentle shaking. Polyclonal anti-Spike protein primary antibodies were then added to the blots (1:1000 dilution) and incubated overnight at 4° C. in PBS-5% BSA, followed by five times rinsing in PBST buffer (1×PBS and 0.05% Tween 20). The following day, the goat-anti-mouse secondary antibody, an HRP-conjugated, was applied at 1:2000 dilution in 5% BSA-PBST for 1 h at RT with gentle shaking. After rinsing five times in PBST, binding was visualized with an enhanced chemiluminescence substrate using the GE Amersham 680 System and integrated software according to the manufacturer's instructions (GE). Ladder refers to molecular weight markers and their sizes are depicted. Arrows indicate correct size proteins.

FIG. 5 shows expression of transgenes in HEK293 cells. HEK293 cells were grown as described above and infected with 1000 virus particles of construct #s 2, 4, and 6, as well as with Ad5 vector alone. After 30 min of infection, medium was aspirated and replaced with the fresh medium. After 24-48 h of infection, the host cells were harvested with RIPA buffer and briefly sonicated to shear DNA. Cell lysates were diluted 1:10 in PBS, and 10 μL was added to 10 μL of 4×SDS-PAGE loading buffer with â-mercaptoethanol. Samples were boiled for 5 min, loaded to a 4-20% Mini-PROTEAN Bio-Rad gel, and run for 1 hour at 150V before transferring to the PVDF membrane. After transfer, the membrane was blocked with 5% skim milk powder-PBS (pH 7.4) at room temperature (RT) for 1 hour with gentle shaking. Polyclonal anti-Spike protein primary antibodies were then added to the blots (1:1000 dilution) and incubated overnight at 4° C. in PBS-5% BSA, followed by five times rinsing in PBST buffer (1×PBS and 0.05% Tween 20). The following day, the goat-anti-mouse secondary antibody, an HRP-conjugated, was applied at 1:2000 dilution in 5% BSA-PBST for 1 hour at RT. After rinsing five times in PBST, binding was visualized with an enhanced chemiluminescence substrate using the GE Amersham 680 System and integrated software according to the manufacturer's instructions (GE). Ladder refers to molecular weight markers and their sizes are depicted. Arrows indicate correct size proteins.

FIG. 6 shows protective efficacy of Ad5 vaccine candidates in mice. Percentage body weight of immunized mice post-challenge with 105-PFU of SARS-CoV-2 MA10 (intranasal route). At day 4 post infection when mice showed maximum weight loss in the control group. Animals immunized with the Ad5 vaccine candidates lost none to 5% of body weight on day 4. The data were presented as mean±SD.

FIG. 7 shows protective efficacy of Ad5 vaccine candidates in mice. Percentage body weight of immunized mice post-challenge with 10⁵-PFU of SARS-CoV-2 MA10 (intranasal route). Upper panel—animals were immunized with a higher dose of the vaccine. At day 2 post infection when mice showed maximum weight loss in the control group (10-15%). Lower panel—animals were immunized with a lower dose of the vaccine. The Ad5 vaccine candidates lost none to 5% of body weight on day 2. The data were presented as mean.

FIG. 8 shows antibody titers to S, M, and N proteins with various Ad5 constructs. The description of each construct is shown in Table 1.

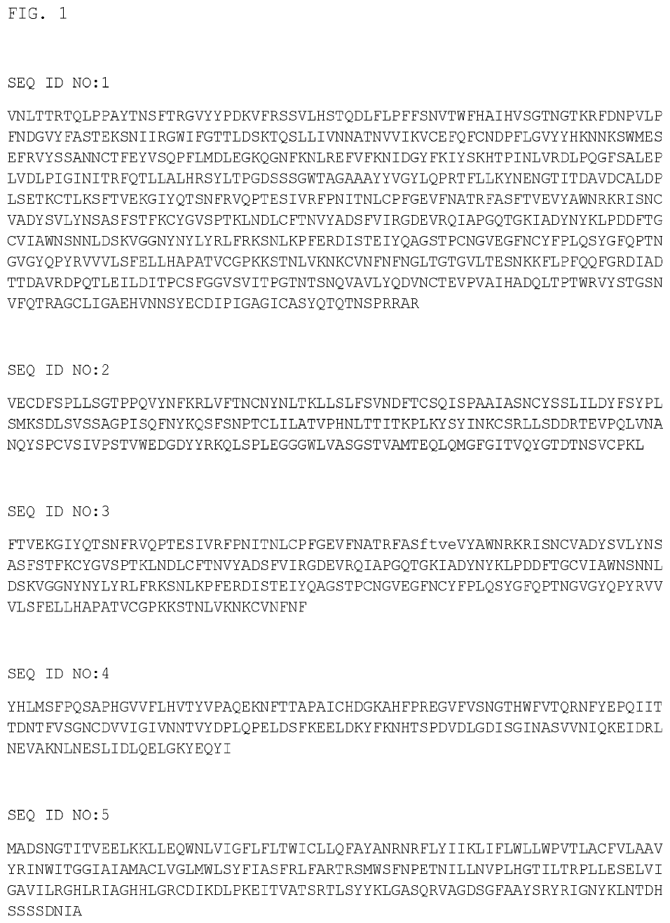

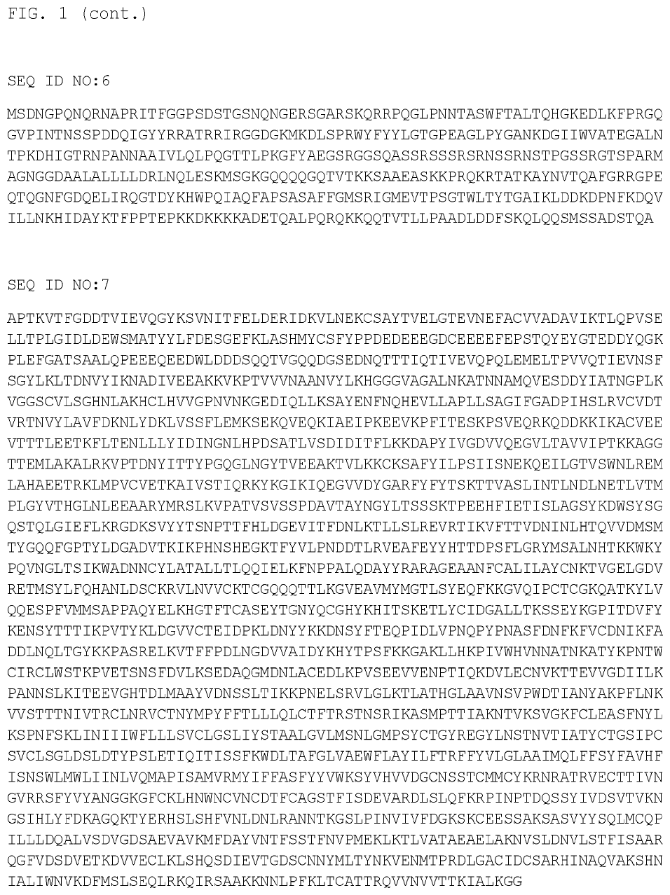

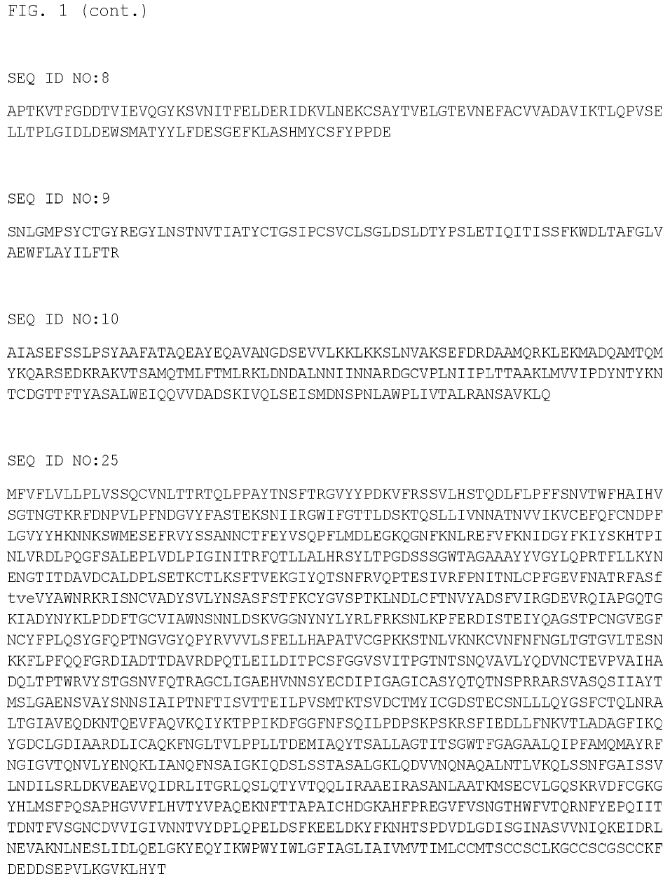

### DETAILED DESCRIPTION

Proteins

Provided herein are proteins and methods for making and using the proteins. A protein described herein can contain from 1 to 5 domains. The domains correspond to different proteins, or a region of a protein, produced by a SARS-CoV-2 or a MERS-CoV. In one embodiment, a domain is a SARS-CoV-2 S1-spike protein (an example of which is shown in SEQ ID NO:1), a MERS S1-RBD region (an example of which is shown in SEQ ID NO:2), a SARS-CoV-2 S1-RBD region (an example of which is shown in SEQ ID NO:3), a SARS-CoV-2 S2-HR2 region (an example of which is shown in SEQ ID NO:4), a SARS-CoV-2 M protein (an example of which is shown in SEQ ID NO:5), a SARS-CoV-2 N protein (an example of which is shown in SEQ ID NO:6), a SARS-CoV-2 Nsp3 protein (an example of which is shown in SEQ ID NO:7), a SARS-CoV-2 Ubl1-Nsp3 region (an example of which is shown in SEQ ID NO:8), a SARS-CoV-2 3Ecto-Nsp3 region (an example of which is shown in SEQ ID NO:9), a SARS-CoV-2 Nsp8 protein (an example of which is shown in SEQ ID NO:10). In one embodiment, the protein includes a domain that is a SARS-CoV-2 full spike protein (an example of which is shown in SEQ ID NO:25).

Other examples of the domains include those having sequence similarity with the amino acid sequence of SEQ ID NO:1, 2, 3, 4, 5, 6, 7, 8, 9, 10, or 25. Unless a specific level of sequence similarity and/or identity is expressly indicated herein (e.g., at least 80% sequence similarity, at least 90% sequence identity, etc.), reference to the amino acid sequence of an identified SEQ ID NO includes variants having the levels of sequence similarity and/or the levels of sequence identity described herein.

A protein of the present disclosure can include any combination of proteins produced by a SARS-CoV-2 virus or a MERS-CoV virus. In one embodiment, the protein includes a combination of a subset of the domains described herein, e.g., SEQ ID NOs:1-10 and SEQ ID NO:25. In one embodiment, the protein includes SEQ ID NO:25. A protein of the present disclosure can be isolated, and optionally purified. In one embodiment, the protein has a first domain that includes the amino acid sequence of a SARS-CoV-2 S1-spike protein and a second domain that includes the amino acid sequence of a MERS S1-RBD region. For example, the first domain can have at least 70% identity to SEQ ID NO:1 and the second domain can have at least 70% identity to SEQ ID NO:2. The order of the domains in the protein can be in the order of first domain-second domain, or second domain-first-domain.

An example of a protein having the amino acid sequence of a SARS-CoV-2 S1-spike protein and the amino acid sequence of a MERS S1-RBD region is shown at SEQ ID NO:11. Amino acids 16-689 are the SARS-CoV-2 S1-spike protein and amino acids 705-912 are the MERS S1-RBD region. Amino acids 1-15 are a leader sequence and amino acids 690-704 are a linker. Leader sequences and linkers are described herein.

In one embodiment, the protein has a first domain that includes the amino acid sequence of a SARS-CoV-2 S1-RBD region, a second domain that includes the amino acid sequence of a SARS-CoV-2 S2-HR2 region, and a third domain that includes the amino acid sequence of a SARS-CoV-2 M protein. For example, the first domain can have at least 70% identity to SEQ ID NO:3, the second domain can have at least 70% identity to SEQ ID NO:4, and the third domain can have at least 70% identity to SEQ ID NO:5. The order of the domains in the protein can be in any order, and in one embodiment are in the order of first domain-second domain-third domain.

An example of a protein having the amino acid sequence of a SARS-CoV-2 S1-RBD region, the amino acid sequence of a SARS-CoV-2 S2-HR2 region, and the amino acid sequence of a SARS-CoV-2 M protein is shown at SEQ ID NO:13. Amino acids 16-257 are the SARS-CoV-2 S1-RBD region, amino acid 273-436 are the SARS-CoV-2 S2-R2 region, and amino acids 452-669 are the SARS-CoV-2 M protein. Amino acids 1-15 are a leader sequence and amino acids 258-272 and 437-451 are linkers.

In one embodiment, the protein has a first domain that includes the amino acid sequence of a SARS-CoV-2 S1-RBD region, a second domain that includes the amino acid sequence of a SARS-CoV-2 S2-HR2 region, and a third domain that includes the amino acid sequence of a SARS-CoV-2 N protein. For example, the first domain can have at least 70% identity to SEQ ID NO:3, the second domain can have at least 70% identity to SEQ ID NO:4, and the third domain can have at least 70% identity to SEQ ID NO:6. The order of the domains in the protein can be in any order, and in one embodiment are in the order of first domain-second domain-third domain.

An example of a protein having the amino acid sequence of a SARS-CoV-2 S1-RBD region, the amino acid sequence of a SARS-CoV-2 S2-HR2 region, and the amino acid sequence of a SARS-CoV-2 N protein is shown at SEQ ID NO:15. Amino acids 16-257 are the SARS-CoV-2 S1-RBD region, amino acids 273-436 are the SARS-CoV-2 S2-HR2 region, and amino acids 452-870 are the SARS-CoV-2 N protein. Amino acids 1-15 are a leader sequence and amino acids 258-272 and 437-451 are linkers.

In one embodiment, the protein has a first domain that includes the amino acid sequence of a SARS-CoV-2 S1-RBD region, a second domain that includes the amino acid sequence of a SARS-CoV-2 S2-HR2 region, a third domain that includes the amino acid sequence of a SARS-CoV-2 M protein, and a fourth domain that includes the amino acid sequence of a SARS-CoV-2 N protein. For example, the first domain can have at least 70% identity to SEQ ID NO:3, the second domain can have at least 70% identity to SEQ ID NO:4, the third domain can have at least 70% identity to SEQ ID NO:5, and the fourth domain can have at least 70% identity to SEQ ID NO:6. The order of the domains in the protein can be in any order, and in one embodiment are in the order of first domain-second domain-third domain-fourth domain.

An example of a protein having the amino acid sequence of a SARS-CoV-2 S1-RBD, the amino acid sequence of a SARS-CoV-2 S2-HR2 region, the amino acid sequence of a SARS-CoV-2 M protein, and the amino acid sequence of a SARS-CoV-2 N protein is shown at SEQ ID NO:17. Amino acids 16-257 are the SARS-CoV-2 S1-RBD, amino acids 273-436 are the SARS-CoV-2 S2-HR2 region, amino acids 452-669 are the SARS-CoV-2 M protein, and amino acids 685-1,103 are the SARS-CoV-2 N protein. Amino acids 1-15 are a leader sequence and amino acids 258-272, 437-451, and 670-684 are linkers.

In one embodiment, the protein has a first domain that includes the amino acid sequence of a SARS-CoV-2 S1-RBD region, and a second domain that includes the amino acid sequence of a SARS-CoV-2 Nsp3 protein. For example, the first domain can have at least 70% identity to SEQ ID NO:3, and the second domain can have at least 70% identity to SEQ ID NO:7. The order of the domains in the protein can be in the order of first domain-second domain, or second domain-first-domain.

An example of a protein having the amino acid sequence of a SARS-CoV-2 S1-RBD region and the amino acid sequence of a SARS-CoV-2 Nsp3 protein is shown at SEQ ID NO:19. Amino acids 16-257 are the SARS-CoV-2 S1-RBD region and amino acids 273-2,217 are the SARS-CoV-2 nsp3 protein. Amino acids 1-15 are a leader sequence and amino acids 258-272 are a linker.

In one embodiment, the protein has a first domain that includes the amino acid sequence of a SARS-CoV-2 S1-RBD region, a second domain that includes the amino acid sequence of a SARS-CoV-2 S2-HR2 region, a third domain that includes the amino acid sequence of a SARS-CoV-2 Ubl1-Nsp3 region, a fourth domain that includes the amino acid sequence of a SARS-CoV-2 3Ecto-Nsp3 region, and a fifth domain that includes the amino acid sequence of a SARS-CoV-2 Nsp8 protein. For example, the first domain can have at least 70% identity to SEQ ID NO:3, the second domain can have at least 70% identity to SEQ ID NO:4, the third domain can have at least 70% identity to SEQ ID NO:8, the fourth domain can have at least 70% identity to SEQ ID NO:9, and the fifth domain can have at least 70% identity to SEQ ID NO:10. The order of the domains in the protein can be in any order, and in one embodiment are in the order of first domain-second domain-third domain-fourth domain-fifth domain.

An example of a protein having the amino acid sequence of a SARS-CoV-2 S1-RBD region, the amino acid sequence of a SARS-CoV-2 S2-HR2 region, the amino acid sequence of a SARS-CoV-2 Ubl1-Nsp3 region, the amino acid sequence of a SARS-CoV-2 3Ecto-Nsp3 region, and the amino acid sequence of a SARS-CoV-2 Nsp8 protein is shown at SEQ ID NO:21. Amino acids 16-257 are the SARS-CoV-2 S1-RBD region, amino acids 273-436 are the SARS-CoV-2 S2-HR2 region, amino acids 452-562 are the SARS-CoV-2 Ubl1-Nsp3 region, amino acids 578-659 are the SARS-CoV-2 3Ecto-Nsp3 region, and amino acids 675-872 are the SARS-CoV-2 Nsp8 protein. Amino acids 1-15 are a leader sequence and amino acids 258-272, 437-451, 563-577, and 660-674 are linkers.

In one embodiment, the protein has a first domain that includes the amino acid sequence of a SARS-CoV-2 S1-spike protein and a second domain that includes the amino acid sequence of a SARS-CoV-2 Nsp8 protein. For example, the first domain can have at least 70% identity to SEQ ID NO:1, and the second domain can have at least 70% identity to SEQ ID NO:10. The order of the domains in the protein can be in the order of first domain-second domain, or second domain-first-domain.

An example of a protein having the amino acid sequence of a SARS-CoV-2 S1-spike protein and the amino acid sequence of a SARS-CoV-2 Nsp8 protein is shown at SEQ ID NO:23. Amino acids 16-689 are the SARS-CoV-2 S1-spike protein and amino acids 705-902 are the SARS-CoV-2 Nsp8 protein. Amino acids 1-15 are a leader sequence and amino acids 690-704 are a linker.

In one embodiment, the protein has a domain that includes the amino acid sequence of a SARS-CoV-2 full spike protein. For example, the domain can have at least 70% identity to SEQ ID NO:25. An example of a protein having the amino acid sequence of a SARS-CoV-2 full spike protein is shown at SEQ ID NO:25. Amino acids 1-15 are a leader sequence.

In one embodiment, the protein has a first domain that includes the amino acid sequence of a SARS-CoV-2 full spike protein, a second domain that includes the amino acid sequence of a SARS-CoV-2 Ubl1-Nsp3 protein, a third domain that includes the amino acid sequence of a SARS-CoV-2 3Ecto-Nsp3 protein, and a fourth domain that includes the amino acid sequence of a SARS-CoV-2 Nsp8 protein. For example, the first domain can have at least 70% identity to SEQ ID NO:25, the second domain can have at least 70% identity to SEQ ID NO:8, the third domain can have at least 70% identity to SEQ ID NO:9, and the fourth domain can have at least 70% identity to SEQ ID NO:10. The order of the domains in the protein can be in any order, and in one embodiment are in the order first domain-second domain-third domain-fourth domain.

An example of a protein having the amino acid sequence of a SARS-CoV-2 full spike protein, the amino acid sequence of a SARS-CoV-2 Ubl1-Nsp3 protein, the amino acid sequence of a SARS-CoV-2 3ecto-Nsp3 protein, and the amino acid sequence of a SARS-CoV-2 Nsp8 is shown at SEQ ID NO:27. Amino acids 16-1277 are the SARS-CoV-2 full spike protein, amino acids 1293-1403 are the SARS-CoV-2 Ubl1-Nsp3 protein, amino acids 1419-1500 are the SARS-CoV-2 3ecto-Nsp3 protein, and amino acids 1516-1713 are the SARS-CoV-2 Nsp8 protein. Amino acids 1-15 are a leader sequence and amino acids 1278-1292, 1404-1418, and 1501-1515 are linkers.

In one embodiment, the protein has a first domain that includes the amino acid sequence of a SARS-CoV-2 S1-spike protein, a second domain that includes the amino acid sequence of a SARS-CoV-2 M protein, a third domain that includes the amino acid sequence of a SARS-CoV-2 N protein, and a fourth domain that includes the amino acid sequence of a SARS-CoV-2 Nsp8 protein. For example, the first domain can have at least 70% identity to SEQ ID NO:1, the second domain can have at least 70% identity to SEQ ID NO:5, the third domain can have at least 70% identity to SEQ ID NO:6, and the fourth domain can have at least 70% identity to SEQ ID NO:10. The order of the domains in the protein can be in any order, and in one embodiment are in the order first domain-second domain-third domain-fourth domain.

An example of a protein having the amino acid sequence of a SARS-CoV-2 S1-spike protein, the amino acid sequence of a SARS-CoV-2 M protein, the amino acid sequence of a SARS-CoV-2 N protein, and the amino acid sequence of a SARS-CoV-2 Nsp8 is shown at SEQ ID NO:29. Amino acids 16-689 are the SARS-CoV-2 S1-spike protein, amino acids 705-922 are the SARS-CoV-2 M protein, amino acids 938-1356 are the SARS-CoV-2 N protein, and amino acids 1372-1569 are the SARS-CoV-2 Nsp8 protein. Amino acids 1-15 are a leader sequence and amino acids 690-704, 923-937, and 1357-1371 are linkers.

In one embodiment, the protein has a first domain that includes the amino acid sequence of a SARS-CoV-2 M protein, a second domain that includes the amino acid sequence of a SARS-CoV-2 N protein, a third domain that includes the amino acid sequence of a SARS-CoV-2 Ubl1-Nsp3 protein, a fourth domain that includes the amino acid sequence of a SARS-CoV-2 3Ecto-Nsp3 protein, and a fifth domain that includes the amino acid sequence of a SARS-CoV-2 Nsp8 protein. For example, the first domain can have at least 70% identity to SEQ ID NO:5, the second domain can have at least 70% identity to SEQ ID NO:6, the third domain can have at least 70% identity to SEQ ID NO:7, the fourth domain can have at least 70% identity to SEQ ID NO:9, and the fifth domain can have at least 70% identity to SEQ ID NO:10. The order of the domains in the protein can be in any order, and in one embodiment are in the order first domain-second domain-third domain-fourth domain-fifth domain.

An example of a protein having the amino acid sequence of a SARS-CoV-2 M protein, the amino acid sequence of a SARS-CoV-2 N protein, the amino acid sequence of a SARS-CoV-2 Ubl1-Nsp3 protein, the amino acid sequence of a SARS-CoV-2 3Ecto-Nsp3 protein, and the amino acid sequence of a SARS-CoV-2 Nsp8 protein is shown at SEQ ID NO:31. Amino acids 1-218 are the SARS-CoV-2 M protein, amino acids 234-652 are the SARS-CoV-2 N protein, amino acids 668-778 are the SARS-CoV-2 Ubl1-Nsp3 protein, amino acids 794-875 are the SARS-CoV-2 3ecto-Nsp3 protein, and amino acids 891-1088 are the SARS-CoV-2 Nsp8 protein. Amino acids 219-233, 653-667, 779-793, and 876-890 are linkers.

A protein described herein has immunological activity. “Immunological activity” refers to the ability of a protein to elicit an immunological response in a subject. An immunological response to a protein is the development in a subject of a cellular and/or humoral, e.g., antibody-mediated, immune response to the protein. Usually, an immunological response includes but is not limited to one or more of the following effects: the production of antibodies, B cells, helper T cells, suppressor T cells, and/or cytotoxic T cells, directed to an epitope or epitopes of the protein. “Epitope” refers to the site on an antigen to which specific B cells and/or T cells respond. The immunological activity may be protective. “Protective immunological activity” refers to the ability of a protein to elicit an immunological response in a subject that prevents or inhibits infection by a coronavirus, such as SARS-CoV-2. Whether a protein has protective immunological activity can be determined by methods known in the art such as, for example, the methods described in Example 1. For example, a protein described herein, or combination of proteins described herein, protects a subject against challenge with a SARS-CoV-2 virus.

Sequence similarity of two proteins can be determined by aligning the residues of the two proteins (for example, a candidate protein domain and a reference protein, e.g., one of SEQ ID NO:1, 2, 3, 4, 5, 6, 7, 8, 9, 10, or 25) to optimize the number of identical amino acids along the lengths of their sequences; gaps in either or both sequences are permitted in making the alignment in order to optimize the number of identical amino acids, although the amino acids in each sequence must nonetheless remain in their proper order. A reference protein may be a protein described herein. A candidate protein is the protein being compared to the reference protein. A candidate protein may be isolated, for example, from a virus such as a SARS-CoV-2 or a MERS-CoV, or can be produced using recombinant techniques, or chemically or enzymatically synthesized. When the candidate protein includes more than one domain, only those amino acids of the protein domain are compared with a reference protein. For instance, if the candidate protein includes a SARS-CoV-2 S1-spike protein, only those residues of a SARS-CoV-2 S1-spike protein domain of the protein are aligned with a reference protein.

Unless modified as otherwise described herein, a pair-wise comparison analysis of amino acid sequences can be carried out using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on. Alternatively, proteins may be compared using the BESTFIT algorithm in the GCG package (version 10.2, Madison WI).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a protein described herein may be selected from other members of the class to which the amino acid belongs. For example, it is known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice-a-versa to maintain a positive charge; Glu for Asp and vice-a-versa to maintain a negative charge; Ser for Thr so that a free —OH is maintained; and Gln for Asn to maintain a free —NH₂.

Guidance concerning how to make phenotypically silent amino acid substitutions is provided in Bowie et al. (1990, Science, 247:1306-1310), wherein the authors indicate proteins are surprisingly tolerant of amino acid substitutions. For example, Bowie et al. disclose that there are two main approaches for studying the tolerance of a protein sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As stated by the authors, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

Guidance on how to modify the amino acid sequences of the protein domains disclosed herein can also be obtained by producing a protein alignment of a reference protein (e.g., SEQ ID NO:1, 2, 3, 4, 5, 6, 7, 8, 9, 10, or 25) with other related polypeptides. For instance, the reference protein SEQ ID NO:1 can be aligned in a multiple protein alignment with other SARS-CoV-2 S1-spike proteins. Such an alignment shows the locations of residues that are identical between each of the proteins, the locations of residues that are conserved between each of the proteins, and the locations of residues that are not conserved between each of the proteins. The identification of identical, conserved, and non-conserved regions and individual amino acids is indicative of correlation between structure and function. By reference to such an alignment, the skilled person can predict which alterations to an amino acid sequence are likely to modify activity, as well as which alterations are unlikely to modify activity. Methods for producing multiple protein alignments are routine, and algorithms such as ClustalW (Larkin et al., 2007, ClustalW and ClustalX version 2, Bioinformatics 23(21): 2947-2948) and Clustl Omega (Sievers et al., 2011, Molecular Systems Biology 7: 539, doi:10.1038/msb.2011.75; Goujon et al., 2010, Nucleic acids research 38 (Suppl 2):W695-9, doi:10.1093/nar/gkg313) are readily available.

Thus, as used herein, a candidate protein domain useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% amino acid sequence identity, or complete identity to a reference amino acid sequence, e.g., SEQ ID NO:1, 2, 3, 4, 5, 6, 7, 8, 9, 10, or 25.

In one embodiment, a protein described herein includes a linker between one or more of the protein domains. A linker is an amino acid sequence that joins protein domains in a protein. A linker can be flexible or rigid, and in one embodiment is flexible. In one embodiment, a linker can be at least 1, at least 2, at least 3, at least 4, at least 5, or at least 6 amino acids in length. It is expected that there is no upper limit on the length of a linker used in a protein described herein; however, in one embodiment, a linker is no greater than 20, no greater than 19, no greater than 18, no greater than 17, or no greater than 16 amino acids in length. Many linkers are known to a skilled person (see Chen et al. 2013, Adv, Drug Deliv. Rev., 65(10):1357-1369). Specific examples of linkers include GGGGSGGGGSGGGGS (SEQ ID NO:33). In one embodiment, a protein can include more than one type of linker, e.g., one type of linker between a first domain and a second domain, and another type of linker between a second domain and a third domain.

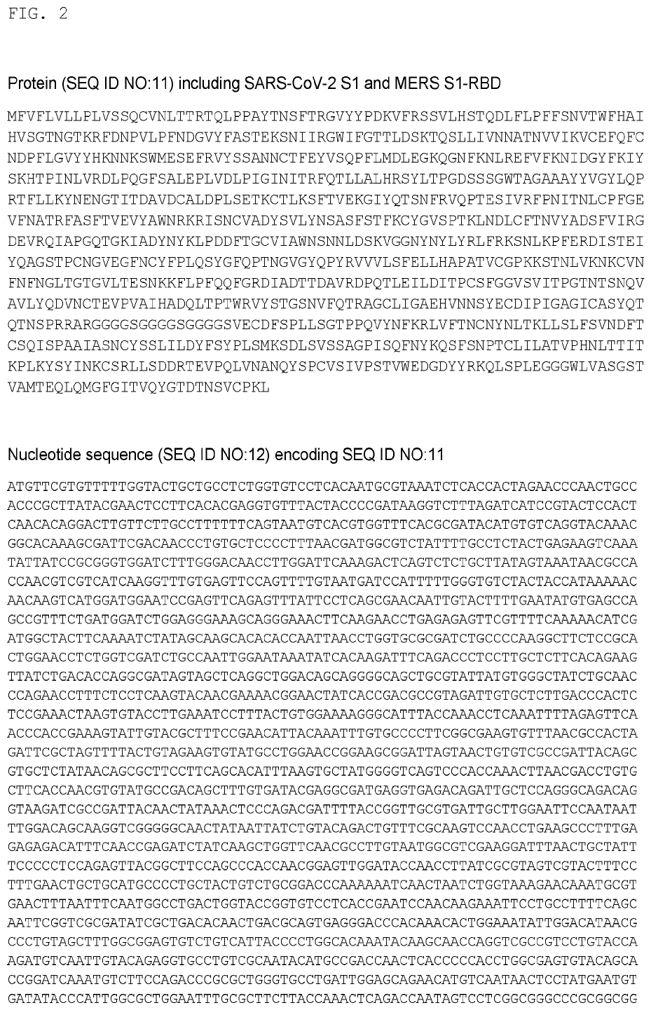

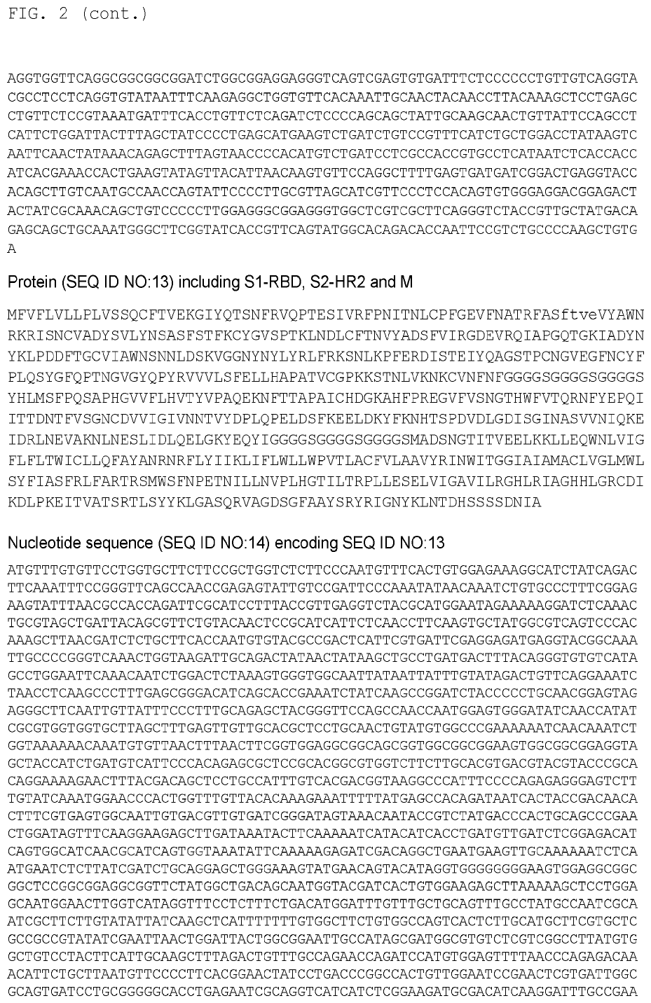

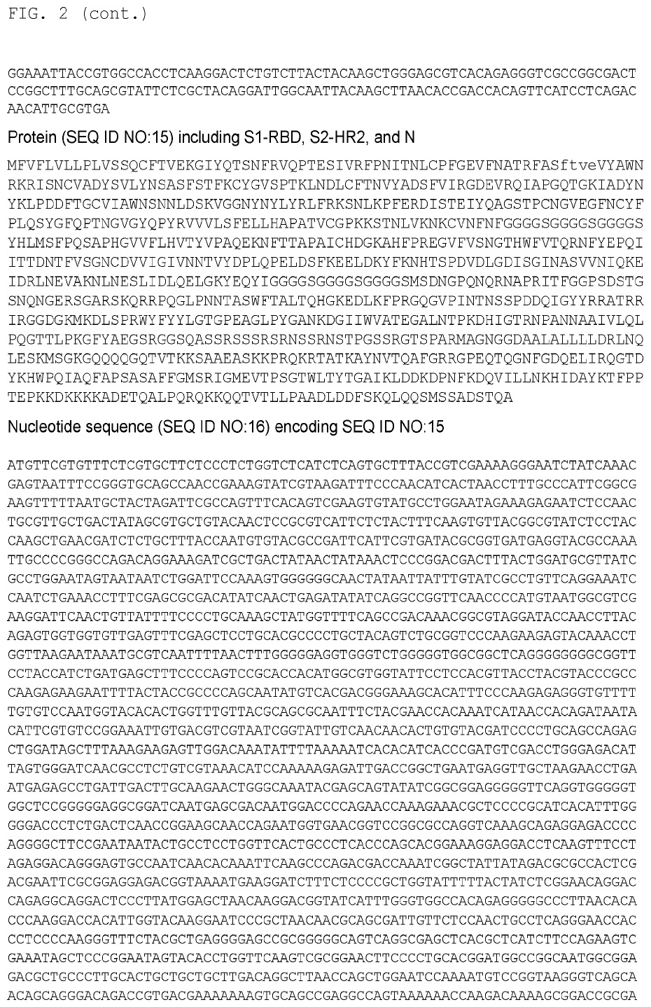

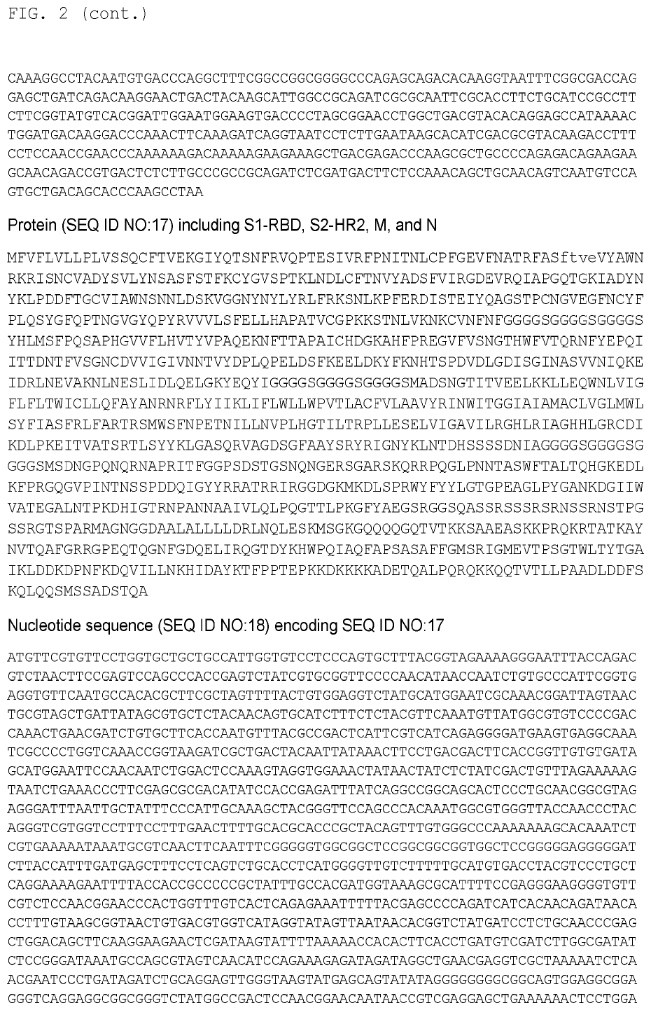

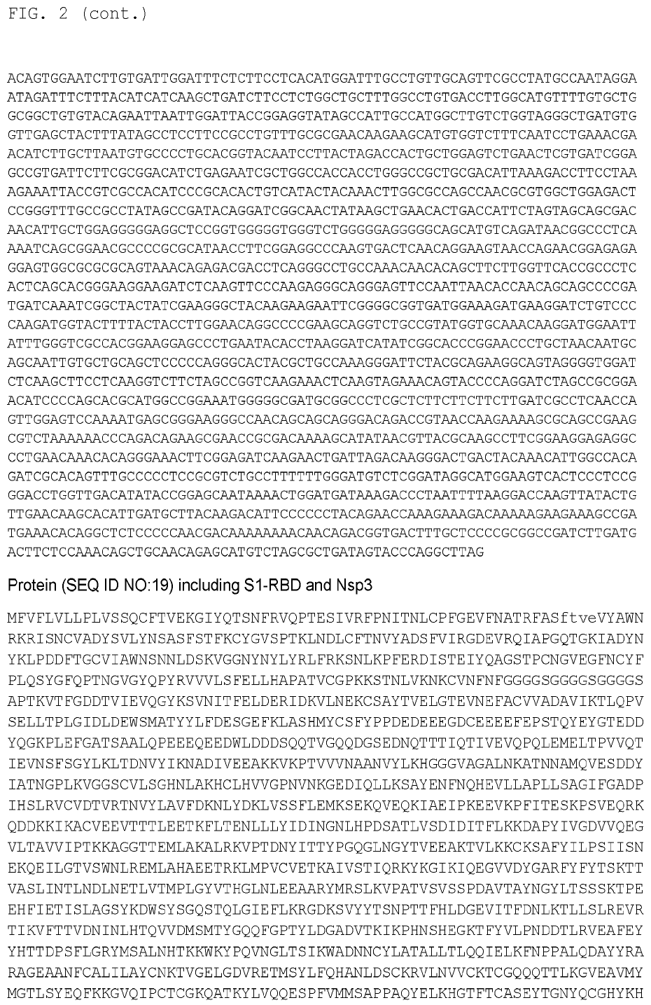

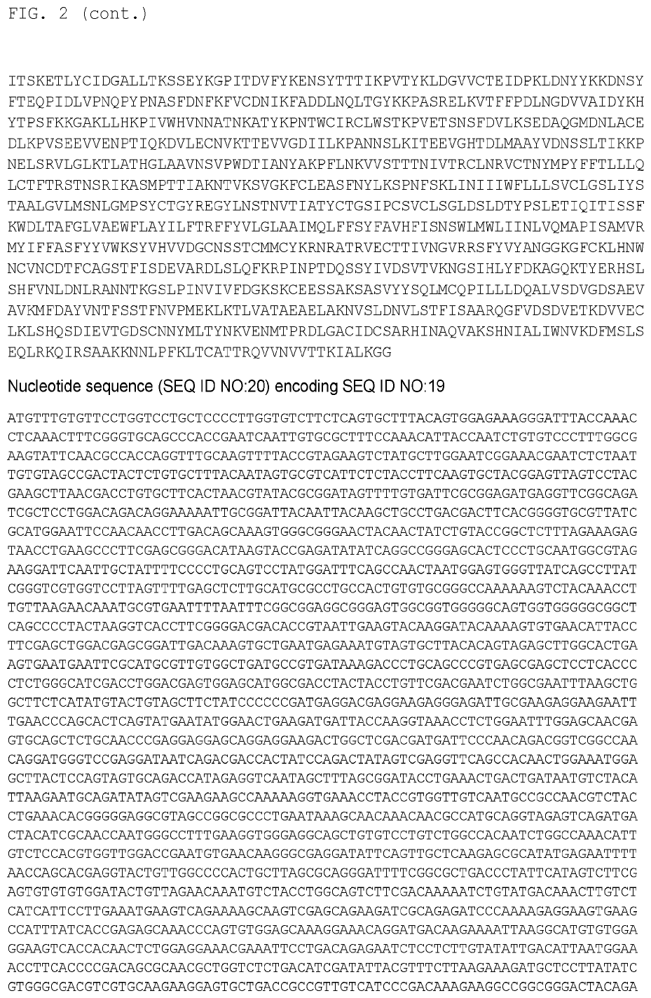

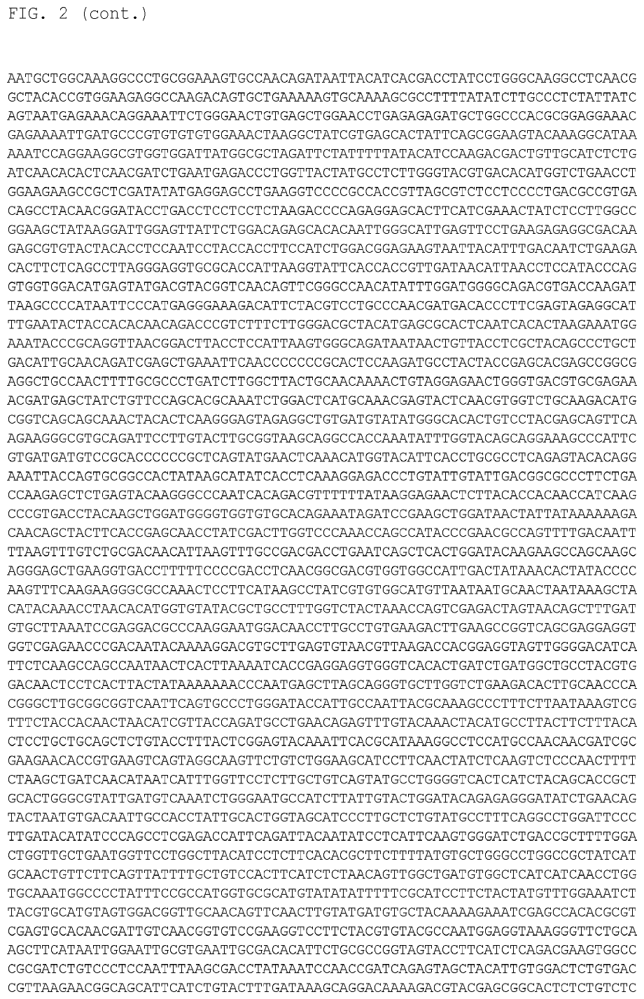

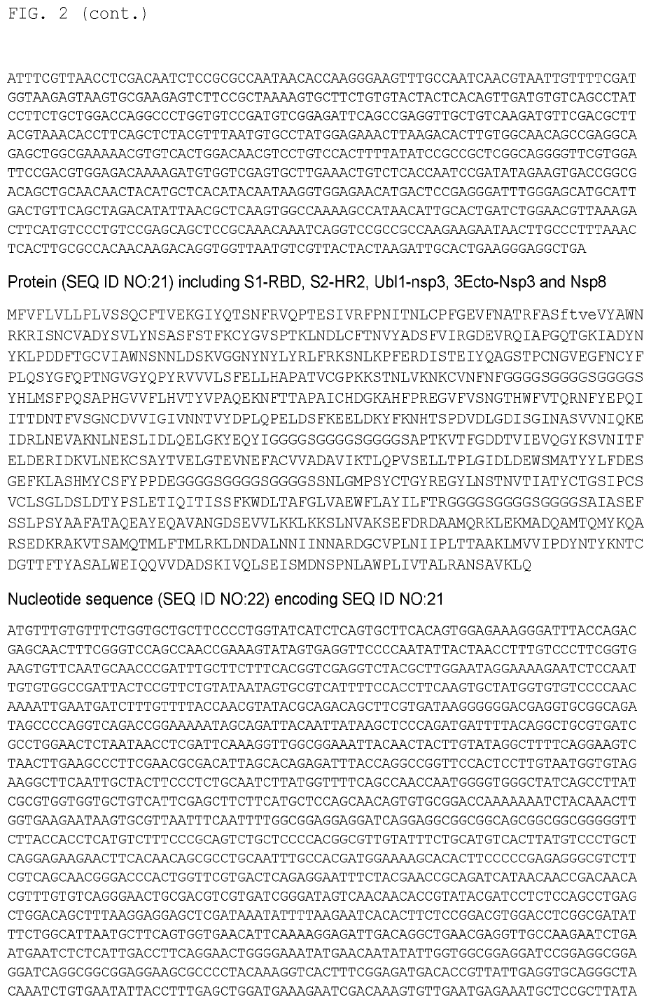

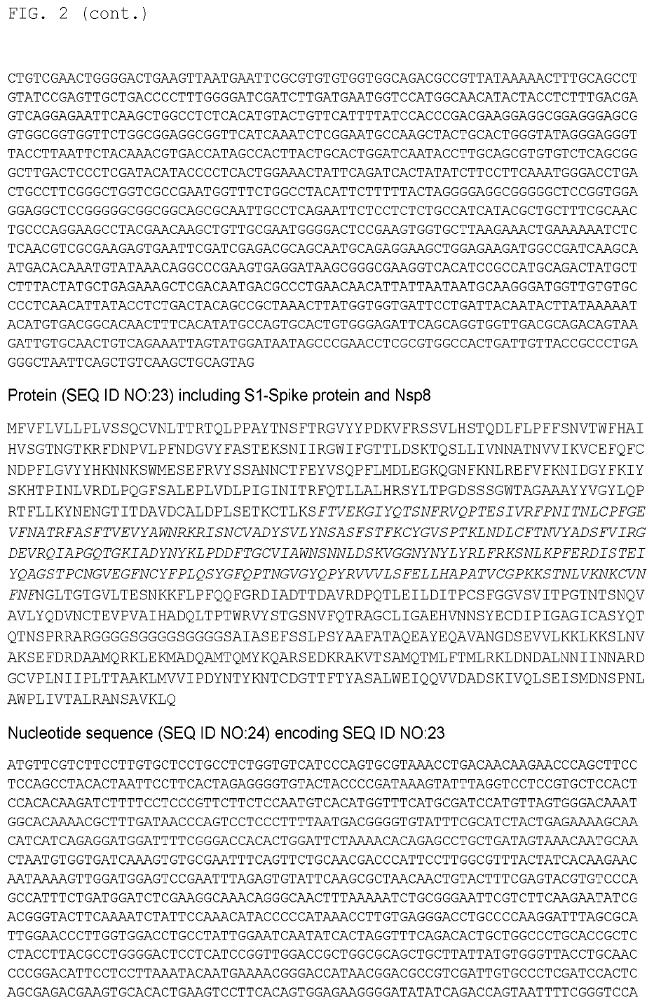

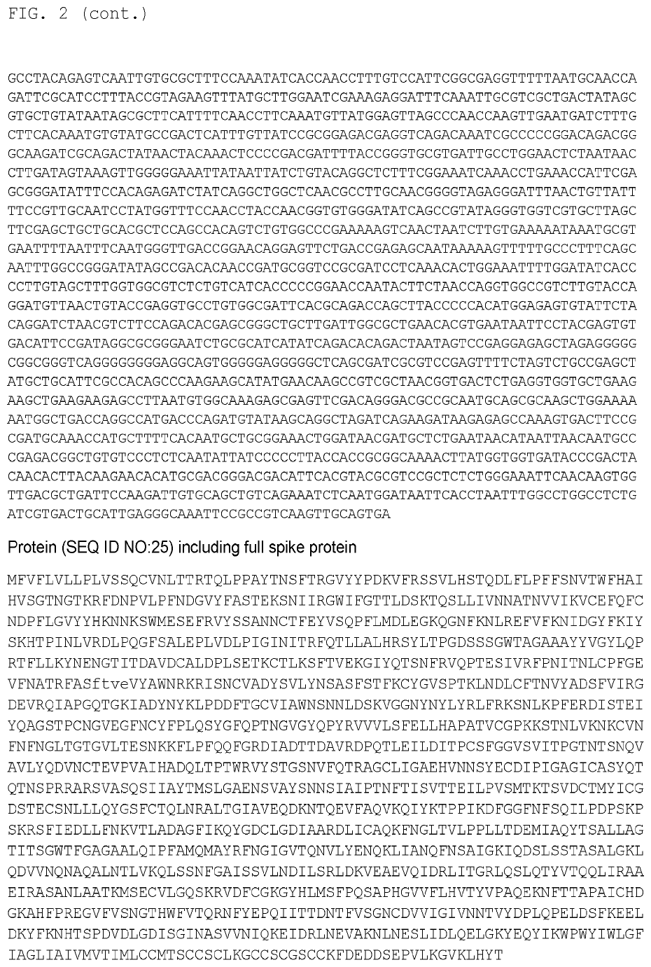

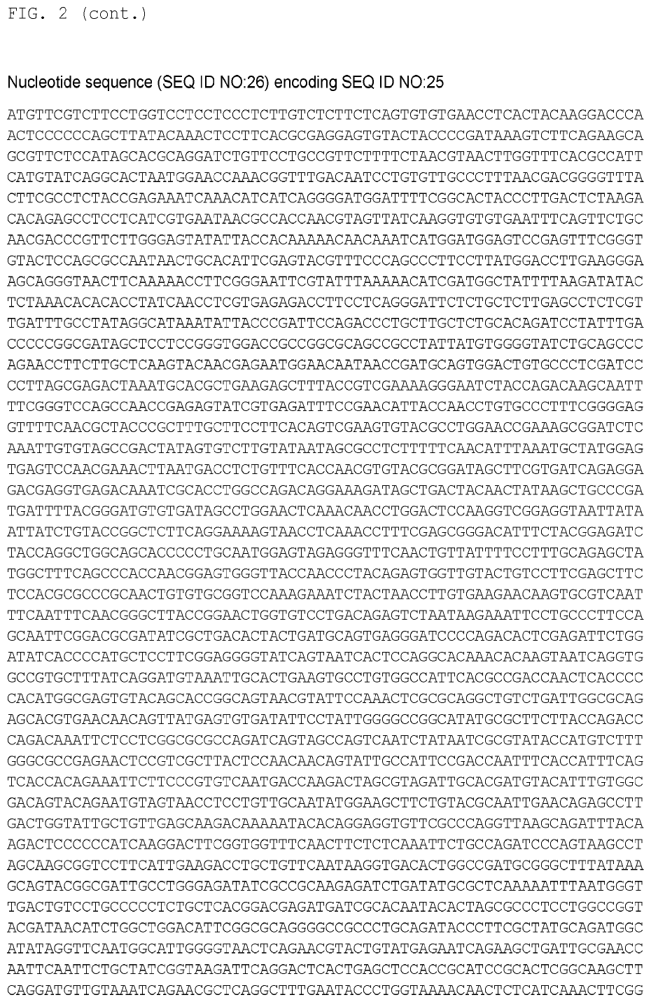

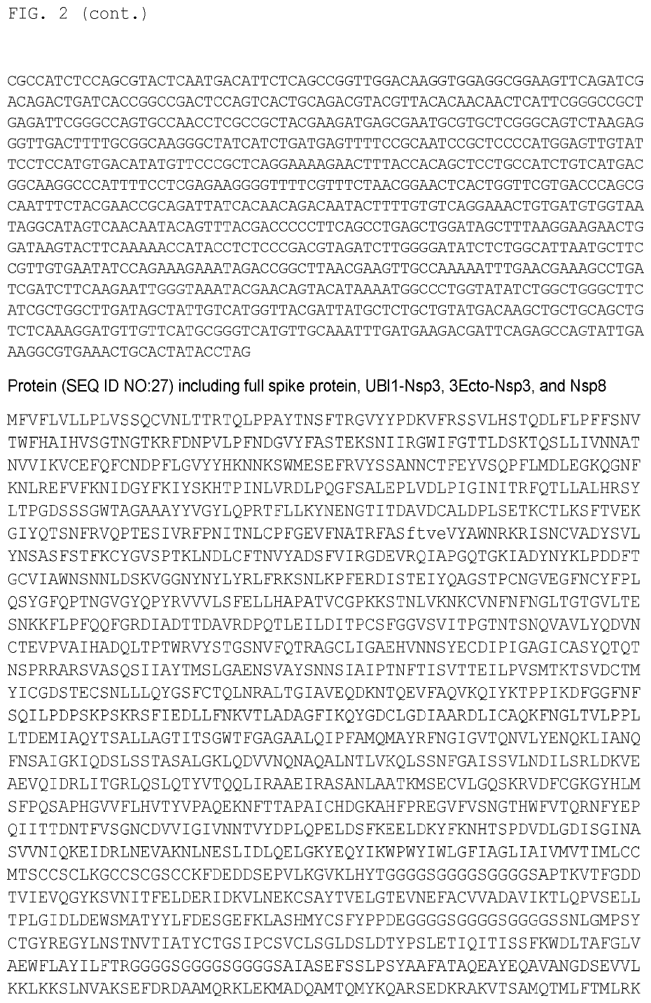

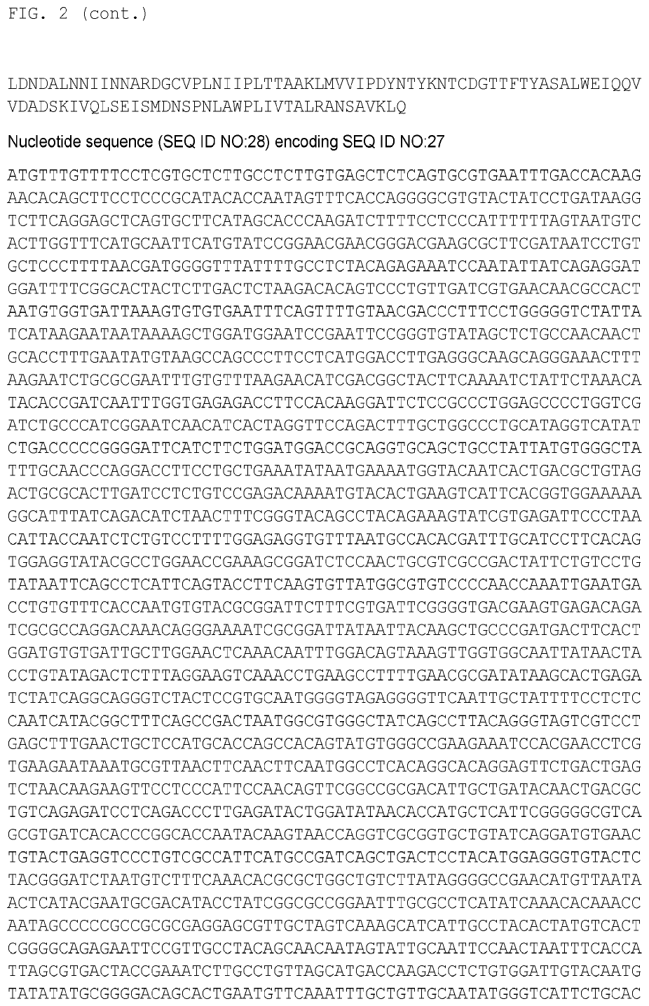

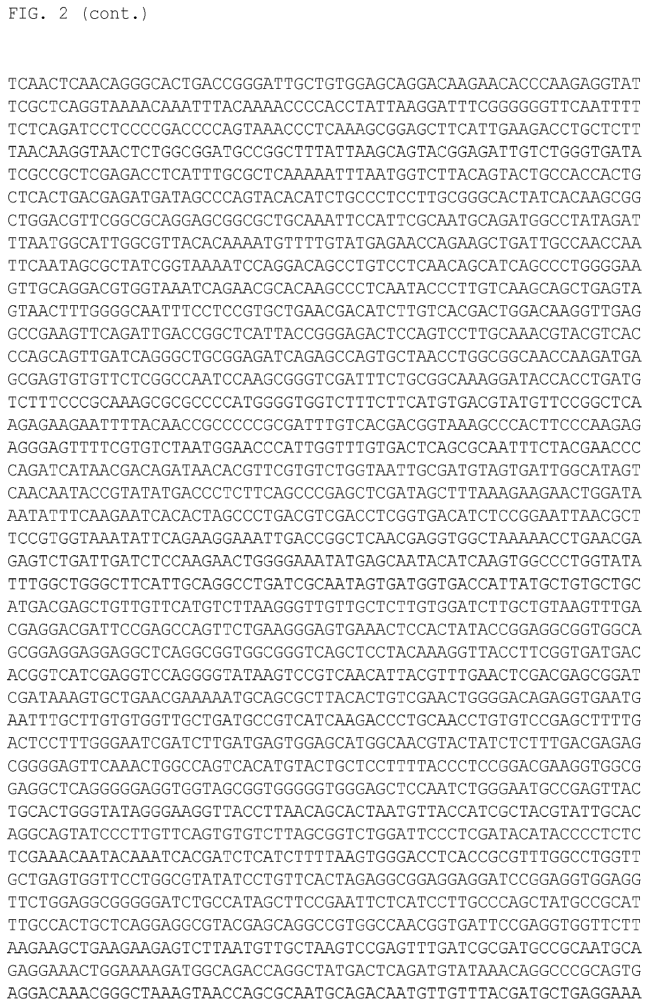

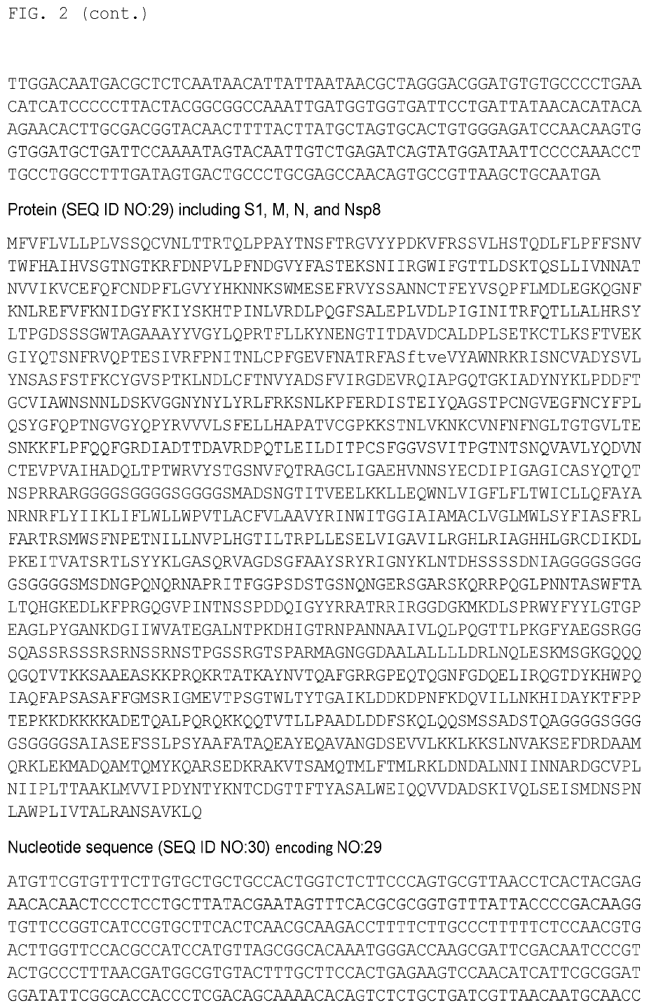

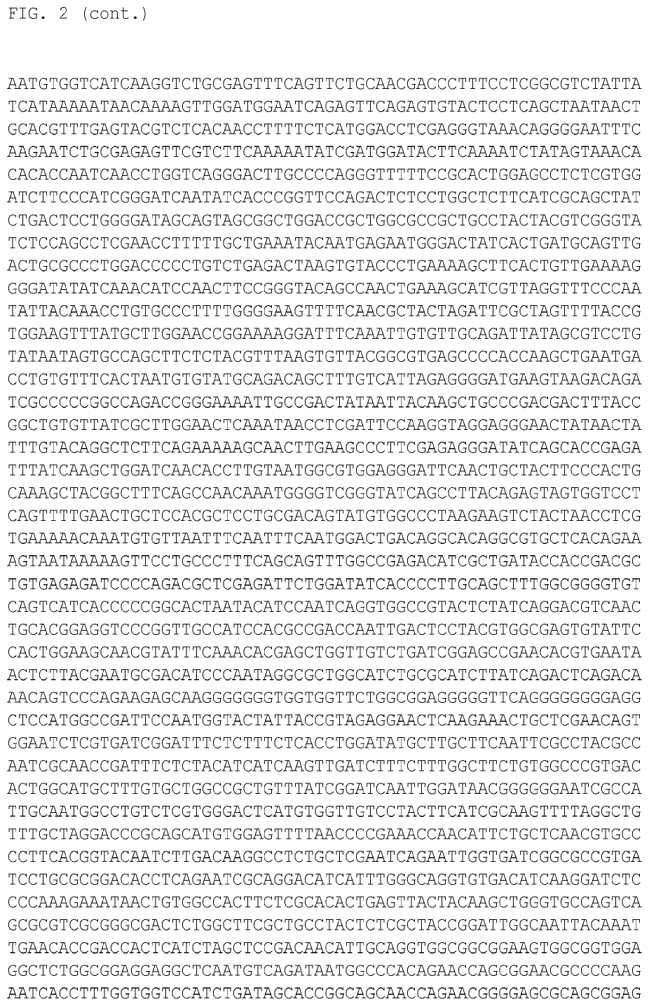

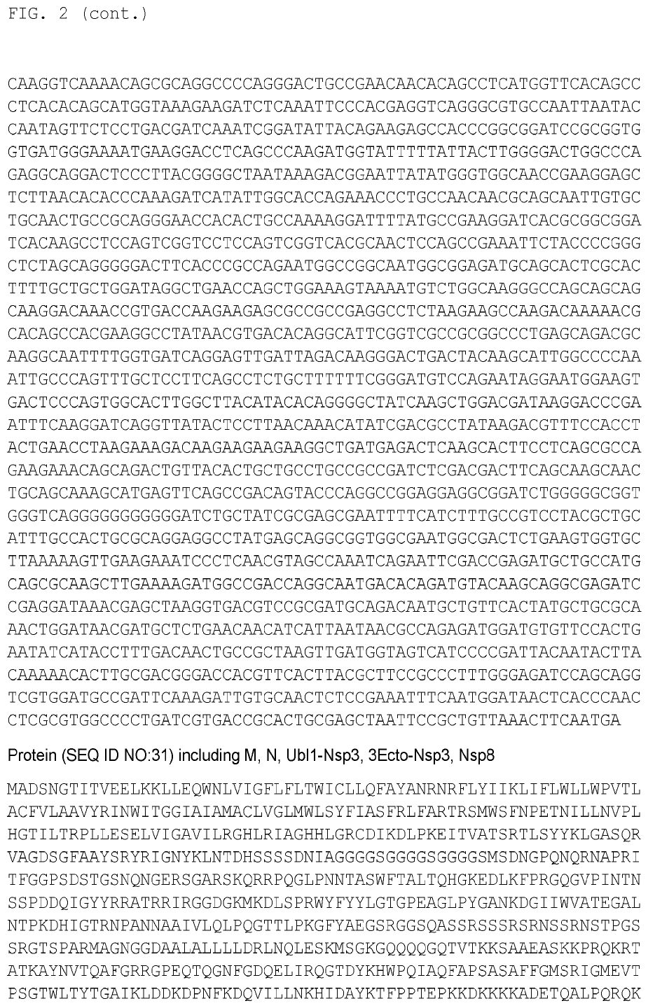

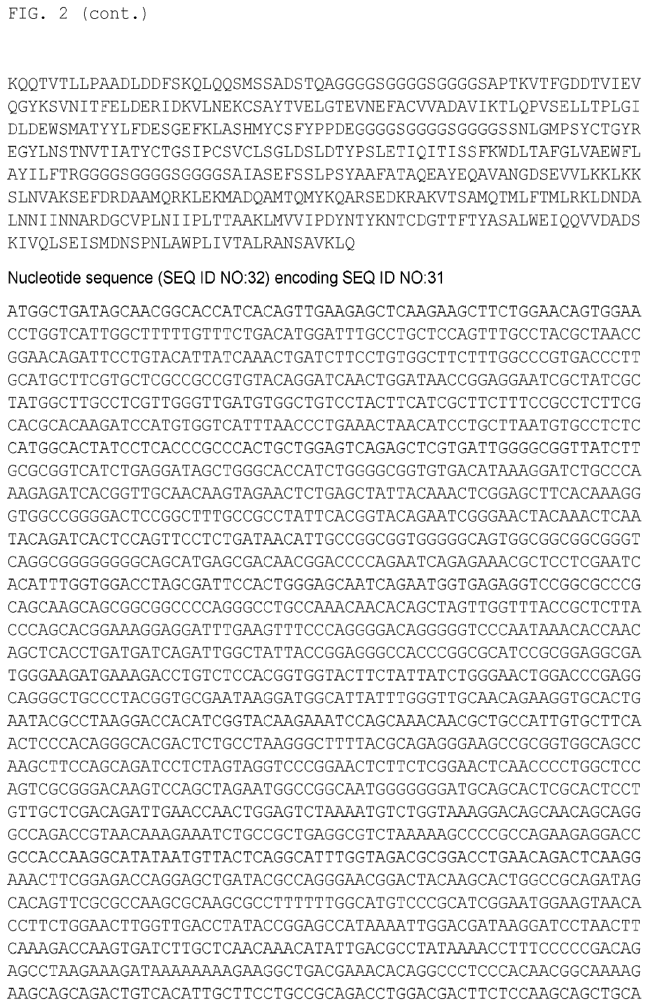

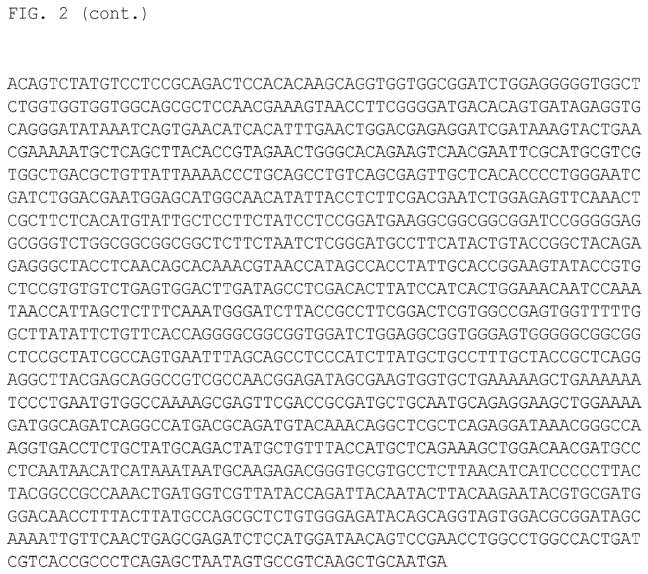

A protein as described herein also can be designed to include one or more additional sequences such as, for example, the addition of C-terminal and/or N-terminal amino acids. In one embodiment, additional amino acids may facilitate purification by trapping on columns or use of antibodies. Such additional amino acids include, for example, histidine-rich tags that allow purification of proteins on nickel columns. In another embodiment, additional amino acids are present at the amino terminal end of the protein and act as a signal to target the protein for export out of the cell in which it is being expressed. This type of amino acid sequence is typically referred to as a leader sequence, signal sequence, and other terms. Some of the proteins shown in FIG. 2 each include the same leader sequence; however, the proteins of the present disclosure are not limited by the leader sequence that may be present.

Polynucleotides

Also provided by the present disclosure are polynucleotides encoding a protein described herein. The polynucleotide can be DNA, RNA, or a combination thereof. Given the amino acid sequence of a protein described herein, a person of ordinary skill in the art can determine the full scope of polynucleotides that encode that amino acid sequence using conventional, routine methods. The class of nucleotide sequences encoding a selected protein sequence is large but finite, and the nucleotide sequence of each member of the class may be readily determined by one skilled in the art by reference to the standard genetic code, wherein different nucleotide triplets (codons) are known to encode the same amino acid. Examples of nucleotide sequences encoding embodiments of proteins described herein are shown in FIG. 2.

In one embodiment, a polynucleotide is a mRNA. A mRNA that includes a polynucleotide encoding a protein disclosed herein and useful as a vaccine typically includes a 5′ cap structure and a 3′ region, each of which aid in translation stability and mRNA stability, and modified nucleosides to aid in stability and translation, and reduce a subject's innate immune response to the mRNA (Pardi et al., 2018, Nature Reviews-Drug Discovery, 17:261-279; U.S. Pat. Nos. 10,703,789; 10,702,600; 10,577,403; 10,442,756; 10,266,485; 10,064,959; 9,868,692). In one embodiment, the mRNA is complexed with a carrier, such as a lipid carrier.

A polynucleotide encoding a protein described herein, may include additional nucleotides flanking the coding region encoding the protein. The boundaries of a coding region are generally determined by a translation start codon at its 5′ end and a translation stop codon at its 3′ end. In one embodiment, the additional nucleotides include a 5′ cap structure and a 3′ region typical of a mRNA for use as a vaccine. In one embodiment, the additional nucleotides include vector nucleotides. In another embodiment, the additional nucleotides aid in expression of the protein, such as expression for subsequent isolation and optional purification.

A polynucleotide that encodes a protein described herein can be present in a vector. A vector is a replicating polynucleotide, such as a plasmid, phage, or cosmid, to which another polynucleotide may be attached so as to bring about the replication of the attached polynucleotide. Construction of vectors containing a polynucleotide described herein employs standard ligation techniques known in the art. A vector can provide for further cloning (amplification of the polynucleotide), e.g., a cloning vector, or for expression of the polynucleotide, e.g., an expression vector. The term vector includes, but is not limited to, plasmid vectors, viral vectors, cosmid vectors, and transposon vectors. Non-limiting examples of viral vectors include, but are not limited to, an adenovirus vector, a poxvirus vector, an alphavirus vector, a retrovirus vector, a vaccinia virus vector, and a lentivirus vector. A vector may be replication-proficient or replication-deficient. A vector may result in integration into a cell's genomic DNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include mammalian cells, such as yeast cells, murine cells, and human cells. Suitable prokaryotic cells include eubacteria, such as Gram-negative organisms, for example, E. coli. Suitable eukaryotic cells include, but are not limited to, human embryonic kidney 293 (HEK293) cells.

An expression vector optionally includes regulatory sequences operably linked to a polynucleotide encoding the protein. An example of a regulatory sequence is a promoter. A promoter may be functional in a host cell used, for instance, in the construction and/or characterization of a polynucleotide encoding a protein described herein, and/or may be functional in the ultimate recipient of the vector. A promoter may be inducible, repressible, or constitutive, and examples of each type are known in the art. A polynucleotide encoding a protein described herein may also include a transcription terminator. Suitable transcription terminators are known in the art.

A vector introduced into a host cell optionally includes one or more marker sequences, which typically encode a molecule that inactivates or otherwise detects or is detected by a compound in the growth medium. Certain selectable markers may be used to confirm that the vector is present within the target cell. For example, the inclusion of a marker sequence may render the transformed cell resistant to an antibiotic, or it may confer compound-specific metabolism on the transformed cell. Examples of a marker sequence include, but are not limited to, sequences that confer resistance to kanamycin, ampicillin, chloramphenicol, tetracycline, streptomycin, neomycin, puromycin, hygromycin, DHFR, GPT, zeocin, histidinol, and others.

In one embodiment, the vector is an adenoviral vector. Adenoviruses are non-enveloped viruses 70-90 nm in diameter with an icosahedral capsid. Their genome is linear, double stranded DNA varying between 25-45 kilobases in size with inverted terminal repeats (ITRs) at both termini and a terminal protein attached to the 5′ ends (Russell, 2000, J Gen Virol., 90:1-20). Their genome also encompasses an encapsidation sequence (Psi), early genes, and late genes. The principal early genes are contained in the regions E1, E2, E3 and E4. Of these, the genes contained in the E1 region are required for viral propagation. The principal late genes are contained in the regions L1 to L5.

Adenoviruses have been used as the basis for a variety of vectors which incorporate various coding regions. In each of these constructs, the adenovirus has been modified in such a way as to render it unable to replicate following gene transfer. Thus, available constructs are adenoviruses in which genes of the early region, adenoviral E1, E2A, E2B, E3, E4, or combinations thereof, are deleted and into the sites of which a DNA sequence encoding a desired protein can be inserted. One example of an adenoviral vector routinely used is adenovirus serotype 5 (Ad5). In the first Ad5 vectors, E1 and/or E3 regions were deleted enabling insertion of foreign DNA to the vectors (Danthinne and Imperiale, 2000, Gene Ther., 7:1707-14; see also Rankii et al., U.S. Pat. No. 9,410,129, and Crouset et al., U.S. Pat. No. 6,261,807). Furthermore, deletions of other regions as well as further mutations have provided extra properties to viral vectors. An example of an adenovirus encoding a protein described herein is disclosed in Clarke (US Patent Publication 2010/0209451). Other examples of adenovirus vectors useful in SARS-CoV-2 vaccines include, but are not limited to, Ad26, ChAd (also referred to as ChAdOx1 (Mendonca et al., 2021, npj Vaccines, 6:97). A viral vector, such as an adenoviral vector, can be present as a polynucleotide or as a polynucleotide inside a viral particle. Methods for producing viral particles for administration to a subject are known in the art and include, for instance, growth of a viral vector encoding a protein described herein in a cell line, followed by purification of infectious viral particles.

Also provided by the present disclosure are compositions. In one embodiment, a composition includes at least one protein described herein, such as a protein. In one embodiment, a composition includes polynucleotide encoding a protein described herein. In one embodiment, the polynucleotide is part of a vector, such as a viral vector, for instance an adenovirus vector, and the vector can be present in a viral particle. In one embodiment, the composition the polynucleotide is a mRNA

The compositions as described herein optionally further include a pharmaceutically acceptable carrier. “Pharmaceutically acceptable” refers to a diluent, carrier, excipient, salt, etc., that is compatible with the other ingredients of the composition, and not deleterious to the recipient thereof. Typically, the composition includes a pharmaceutically acceptable carrier when the composition is used as described herein. The compositions as described herein may be formulated in pharmaceutical preparations in a variety of forms adapted to the chosen route of administration, including routes suitable for stimulating an immune response to an antigen. Thus, a composition as described herein can be administered via known routes including, for example, orally, parenterally including intradermal, subcutaneous, intramuscular, intravenous, intraperitoneal, etc., and topically, such as, intranasal, intrapulmonary, intradermal, transcutaneous, and rectally, etc. In one embodiment a composition is formulated for administration to a mucosal surface, such as by administration to the nasal or respiratory mucosa (e.g., via a spray or aerosol), in order to stimulate mucosal immunity, such as production of secretory IgA antibodies, throughout the subject's body.

A polynucleotide, protein, and composition described herein can be referred to as a vaccine. The term “vaccine” as used herein refers to a polynucleotide, protein, or composition that, upon administration to a subject, will result in an immune response to antigens or antigens encoded by a polynucleotide, such as a viral vector of a mRNA, present in the composition and increase the likelihood the recipient is protected against a coronavirus such as SARS-CoV-2. The immune response can be a primary or initial immune response, e.g., an immune response to antigens to which the subject has not been exposed to before. The immune response can be a secondary immune response, e.g., a pre-existing immune response to antigens to which the subject has been exposed to previously. In one embodiment, a polynucleotide, protein, or composition described herein is administered to a subject that has already received a vaccine that resulted in an immune response to one or more SARS-CoV-2 antigens, such as a subject that has received a vaccine including, but not limited to, the Pfizer-BioNTech COVID-19 vaccine, Moderna COVID-19 vaccine, Astra Zeneca COVID-19 vaccine, or Johnson & Johnson COVID-19 vaccine.

A composition of the present disclosure may be administered to a subject in an amount sufficient to result in an immune response. The immune response can be humoral (e.g., antibody is produced), cellular (e.g., T cells are stimulated) or a combination thereof. A composition of the present disclosure may be administered in an amount sufficient to treat certain conditions as described herein. The amount of protein or vector present in a composition as described herein can vary. In one embodiment, a dosage of viral particles containing a vector that encodes a protein described herein can be at least 1×10⁸, at least 5×10⁸, at least 1×10⁹, at least 5×10⁹, or at least 1×10¹⁰ viral particles, and no greater than 1×10¹², no greater than 5×10¹¹, no greater than 1×10¹¹, no greater than 5×10¹⁰, or no greater than 1×10¹⁰ viral particles. In one embodiment, a dosage of viral particles containing a vector that encodes a protein described herein can be at least 1×10⁸, to no greater than 1×10¹². In one embodiment, a dosage of a protein described herein can be at least 0.01 micrograms (μg), at least 0.1 μg, at least 1 μg, or at least 10 μg, and no greater than 20 μg, no greater than 50 μg, or no greater than 100 μg.

Therapeutic efficacy and toxicity of active compounds (e.g., a viral particle or protein as described herein) can be determined by standard pharmaceutical procedures in cell cultures or experimental animals. The data obtained from cell culture assays and animal studies can be used in formulating a range of dosage for use in humans. The dosage of active compounds lies preferably within a range of circulating concentrations that include the ED₅₀ with little or no toxicity. The dosage may vary within this range depending upon the dosage form employed and the route of administration used. A dose may be formulated in animal models to achieve a circulating plasma concentration range that is effective to achieve an immune response. Such information can be used to more accurately determine useful doses in humans.

The formulations may be conveniently presented in unit dosage form and may be prepared by methods well known in the art of pharmacy. Methods of preparing a composition with a pharmaceutically acceptable carrier include the step of bringing the active compound (e.g., a viral particle or protein as described herein) into association with a carrier that constitutes one or more accessory ingredients. In general, the formulations are prepared by uniformly and intimately bringing the active compound into association with a liquid carrier, a finely divided solid carrier, or both, and then, if necessary, shaping the product into the desired formulations.

A composition can also include an adjuvant. An “adjuvant” refers to an agent that can act in a nonspecific manner to enhance an immune response to a particular antigen, thus potentially reducing the quantity of antigen necessary in any given immunizing composition, and/or the frequency of injection necessary in order to generate an adequate immune response to the antigen of interest. Adjuvants may include, for example, IL-1, IL-2, emulsifiers, muramyl dipeptides, dimethyl dioctadecyl ammonium bromide (DDA), avridine, aluminum hydroxide, alum, magnesium hydroxide, oils, saponins, alpha-tocopherol, polysaccharides, emulsified paraffins, ISA-70, RIBI, TLR agonists, and other substances known in the art. It is expected that proteins as described herein will have immunoregulatory activity and that such proteins may be used as adjuvants that directly act as T cell and/or B cell activators or act on specific cell types that enhance the synthesis of various cytokines or activate intracellular signaling pathways. Such proteins are expected to augment the immune response to increase the protective index of the existing composition.

In another embodiment, a composition as described herein including a pharmaceutically acceptable carrier can include a biological response modifier, such as, for example, IL-2, IL-4 and/or IL-6, TNF, IFN-α, IFN-7, and other cytokines that effect immune cells. A composition can also include other components known in the art such as an antibiotic, a preservative, an anti-oxidant, or a chelating agent.

Also provided are methods of using the proteins, polynucleotides, and compositions described herein. The methods include administering to a subject an effective amount of one or more protein, polynucleotide, and/or composition described herein. In one embodiment, the polynucleotide can be present in a vector, such as an adenoviral vector, and in another embodiment the polynucleotide can be a mRNA. The subject can be, for instance, a mammal, including a human, a non-human primate, a murine (such as a mouse or a rat) animal, a hamster, or a ferret. In one embodiment, the animal is a model system that is recognized as correlating to the protective activity of a protein, polynucleotide, and/or composition described herein against a coronavirus, such as SARS-CoV-2, in a human (Chan et al., Clin Infect Dis, (2020); Bao et al., Nature, (2020); Yuan et al., Emerg Microbes Infect 9, 949-961 (2020); Chandrashekar et al., 2020, Science, 369(6505):812-817).

In some aspects, the methods may further include additional administrations (e.g., one, two, three, four, or more additional primary administrations or booster administrations) of the composition to the subject to enhance an initial immune response to achieve a desired protective effect or to stimulate a secondary immune response. An additional primary administration or booster can be administered at a time after the first administration, for instance, one to eight weeks, such as two to four weeks, after the first administration of the composition. In one embodiment, a booster can be used to sustain a subject's immune response. Subsequent additional primary administrations or boosters can be administered one, two, three, four, or more times annually. An additional primary administration or booster administration can use the same route as the initial administration, or use a different route. For instance, the initial administration can be intranasal, and an additional primary administration or a booster administration can be intranasal or intramuscular. Conversely, an initial administration can be intramuscular or any other method of delivery, followed by additional primary administrations or boosters, which can be intranasal or any other method or combination of methods of delivery. An additional administration can be a different dosage form than the initial administration. For instance, an initial administration can be a mRNA-based composition such as the Pfizer-BioNTech COVID-19 vaccine or the Moderna COVID-19 vaccine, and the additional administration can be a composition of the present disclosure. Likewise, booster administrations can be a different dosage form than an additional primary administration. For instance, the initial administration can be a viral vector and an additional primary administration and/or booster can be a composition that includes viral vector and/or a protein. Without intending to be limited by theory, it is expected that in some aspects annual boosters will or may not be necessary or desired, as a subject will be challenged by exposure to virus expressing proteins having epitopes that are identical to or structurally related to epitopes present on proteins administered to, or expressed in, the subject.

In one embodiment, a method includes an administration of a vector that includes a coding region encoding a protein described herein. The vector can be a viral vector, and the viral vector can be present in a viral particle. In one embodiment, a viral vector is an adenovirus. In one embodiment, the administration of the vector is topical, such as delivery to the nasal or respiratory mucosa. In one embodiment, the administration of the vector can be followed by one or more additional administrations. In one embodiment, the vector can follow administration of another vaccine, such as a mRNA vaccine. The one or more additional administrations can be topical or parenteral, such as intramuscular, intradermal, or subcutaneous. Optionally, more than one administration of the vector can occur.

In one embodiment, a method includes an administration of a mRNA encoding a protein described herein. In one embodiment, the administration of the vector is parenteral, such as intramuscular. In one embodiment, the administration of the mRNA can be followed by one or more additional administrations. In one embodiment, the mRNA can follow administration of another vaccine, such as a vector, including one described herein. The one or more additional administrations can be topical or parenteral, such as intramuscular, intradermal, or subcutaneous. Optionally, more than one administration of the mRNA can occur.

For example, methods of using the proteins, polynucleotides, and compositions described herein include administering to a subject an effective amount of one or more protein, polynucleotide, and/or composition described herein as a single dose. The single dose can be a first administration used to initiate an immune response (e.g., it can be the first immunizing composition administered to a subject) or can be an additional administration (e.g., an additional primary administration or booster administration) after a subject has already received a dose of a composition that has initiated an immune response (e.g., a protein, polynucleotide, or composition described herein, or a different vaccine such as the Pfizer-BioNTech, Moderna, Astra Zeneca, or Johnson & Johnson COVID-19 vaccine). In one embodiment, the administration of a single dose of a composition described herein can be intranasal or intramuscular. In one embodiment, the first administration can be more than one of the proteins, polynucleotides, and/or compositions described herein combined in a single dose, for instance two of the proteins, polynucleotides, and/or compositions described herein combined in a single dose. In one embodiment, the additional administration can be more than one of the proteins, polynucleotides, and/or compositions described herein combined in a single dose, for instance two of the proteins, polynucleotides, and/or compositions described herein combined in a single dose.

In another example, methods of using the proteins, polynucleotides, and compositions described herein include administering to a subject an effective amount of one or more protein, polynucleotide, and/or composition described herein as two or more doses, where the same one or more protein, polynucleotide, and/or composition is administered in each dose. The first dose can be a first administration used to initiate an immune response (e.g., it can be the first immunizing composition administered to a subject), and the second dose can be an additional administration (e.g., an additional primary administration or booster administration) after the first dose. In one embodiment, the first dose can be intranasal or intramuscular, and the additional administration can be intranasal or intramuscular. In one embodiment, the first dose can be intranasal and the additional administration can be intramuscular. In one embodiment, the first dose can be intramuscular and the additional administration can be intranasal. In one embodiment, the first dose and the additional administration can be more than one of the proteins, polynucleotides, and/or compositions described herein combined in a single dose, for instance two of the proteins, polynucleotides, and/or compositions described herein combined in a single dose.

In another example, methods of using the proteins, polynucleotides, and compositions described herein include administering to a subject an effective amount of one or more protein, polynucleotide, and/or composition described herein as two or more doses, where the each of the two or more doses include a different one or more protein, polynucleotide, and/or composition. The first dose can be a first administration used to initiate an immune response (e.g., it can be the first immunizing composition administered to a subject), and the second dose can be an additional administration (e.g., an additional primary administration or booster administration) after the first dose. In one embodiment, the first dose can be intranasal or intramuscular, and the additional administration can be intranasal or intramuscular. In one embodiment, the first dose can be intranasal and the additional administration can be intramuscular. In one embodiment, the first dose can be intramuscular and the additional administration can be intranasal. In one embodiment, the first dose and the additional administration can be more than one of the proteins, polynucleotides, and/or compositions described herein combined in a single dose, for instance two of the proteins, polynucleotides, and/or compositions described herein combined in a single dose.

In another example, methods of using the proteins, polynucleotides, and compositions described herein include administering to a subject an effective amount of one or more protein, polynucleotide, and/or composition described herein as two doses that are administered at approximately the same time, but by different or similar routes. For instance, a first dose can be intranasal and the second dose can be intramuscular or intranasal. For instance, a first dose can be intramuscular and the second dose can be intramuscular or intranasal. The two doses can include the same one or more protein, polynucleotide, and/or composition is administered in each dose, or the two doses can include different one or more proteins, polynucleotides, and/or compositions. The two co-administered doses can be a first administration used to initiate an immune response (e.g., it can be the first immunizing composition administered to a subject), or the two co-administered doses can be an additional administration (e.g., an additional primary administration or booster administration) after a first administration.

In one aspect, the disclosure is directed to methods for producing an immune response in the recipient subject. An immune response can be humoral, cellular, or a combination thereof. Antibody produced includes antibody that specifically binds a protein of the present disclosure. A cellular immune response includes immune cells that are activated by a protein of the present disclosure. In this aspect, an “effective amount” is an amount effective to result in the production of an immune response in the subject. Methods for determining whether a subject has produced antibodies that specifically bind a protein, and determining the presence of a cellular immune response, are routine and known in the art.

In one aspect the disclosure is also directed to conferring immunity to a coronavirus, such as a member of the genus Betacoronavirus. Due to similar homology, the disclosure can be directed to confer immunity to any strain or mutation of any coronavirus. In one embodiment, the coronavirus is SARS-CoV virus in a subject, including a human. In one embodiment, the coronavirus is SARS-CoV-2 virus in a subject, including a human. In one embodiment, the coronavirus is MERS-CoV virus in a subject, including a human. In other embodiments, the coronavirus is any current or future strain or mutation of coronavirus in a subject, including a human. Conferring immunity is typically prophylactic, e.g., initiated before a subject is infected by the virus, and is referred to herein as treatment of a subject that is “at risk” of infection. As used herein, the term “at risk” refers to a subject that may or may not actually possess the described risk. Thus, typically, a subject “at risk” of infection by the virus is a subject present in an area where subjects have been identified as infected by the virus and/or is likely to be exposed to the virus even if the subject has not yet manifested any detectable indication of infection by the virus and regardless of whether the subject may harbor a subclinical amount of the virus. With respect to SARS-CoV-2, as this virus is the cause of the pandemic that has spread to almost all nations in the world, essentially all humans are at risk. While the methods described herein are of use in prophylactic treatment, the methods can also be used to treat a subject after the subject is infected by the virus. Accordingly, administration of a composition can be performed before, during, or after the subject has first contact with the virus. Treatment initiated before the subject's first contact with the virus can result in increased immunity to infection by the virus.

In another aspect, the method is directed to treating one or more symptoms or clinical signs of certain conditions in a subject that can be caused by infection by a coronavirus such as SARS-CoV-2. As used herein, the term “symptom” refers to subjective evidence of a disease or condition experienced by the patient and caused by infection by a virus. As used herein, the term “clinical sign” or, simply, “sign” refers to objective evidence of disease or condition caused by infection by a virus. The method includes administering an effective amount of a protein, polynucleotide, or composition described herein to a subject having a condition, or exhibiting symptoms and/or clinical signs of a condition, and determining whether at least one symptom and/or clinical sign of the condition is changed, preferably, reduced. Examples of symptoms and/or clinical signs caused by a coronavirus are known to the person skilled in the art. Infection by SARS-CoV-2 can include, but is not limited to, an atypical pneumonia. Individuals with SARS-CoV-2 infection have reported symptoms and/or signs ranging from mild to severe illness. Illness may appear 2 to 14 days after exposure to the virus. Symptoms and/or signs may include fever or chills, cough, shortness of breath, fatigue, muscle or body aches, headache, new loss of taste or smell, sore throat, congestion or runny nose, nausea or vomiting, and diarrhea.

In one embodiment, a method of the present disclosure also include isolating from the subject (i) an antibody that specifically binds to an epitope of one of the proteins, (ii) a nucleic acid encoding an antibody that specifically binds to an epitope of one of the proteins, (iii) a cell comprising a nucleic acid sequence encoding an antibody that specifically binds to an epitope of one of the proteins, or (iv) any immune protective components, with therapeutic effectiveness or specificity against coronavirus, that result from the administrations, that may be isolated, identified, modified, and/or used to diagnose, treat, or prevent coronavirus infections. Such compositions can be used to provide antibodies (polyclonal or monoclonal), nucleic acids, cells, or other immune components which can be used for research, diagnostic, and/or therapeutic purposes according to methods known in the art.

Also provided herein is a kit for immunizing a subject to protect against infection by a coronavirus such as SARS-CoV-2. In one embodiment, the kit includes a vector described herein, such as an adenoviral vector, which includes a coding region encoding a protein described herein in a suitable packaging material in an amount sufficient for at least one administration. In one embodiment, the kit includes a protein described herein, in a suitable packaging material in an amount sufficient for at least one administration. Optionally, other reagents such as buffers and solutions needed to administer the polynucleotide, or the protein are also included. Instructions for use of the packaged materials are also typically included. As used herein, the phrase “packaging material” refers to one or more physical structures used to house the contents of the kit. The packaging material is constructed by known methods, generally to provide a sterile, contaminant-free environment. The packaging material may have a label which indicates that the materials can be used for conferring immunity to a subject. In addition, the packaging material contains instructions indicating how the materials within the kit are employed to immunize a subject to protect against viral infection. As used herein, the term “package” refers to a container such as glass, plastic, paper, foil, and the like, capable of holding within fixed limits the materials and other optional reagents. “Instructions for use” typically include a tangible expression describing the reagent concentration or at least one assay method parameter, such as the relative amounts of reagent and sample to be admixed, maintenance time periods for reagent/sample admixtures, temperature, buffer conditions, and the like.

The invention is defined in the claims. However, below there is provided a non-exhaustive listing of non-limiting exemplary aspects. Any one or more of the features of these aspects may be combined with any one or more features of another example, embodiment, or aspect described herein.

Exemplary Aspects

Aspect 1. A polynucleotide encoding a protein, wherein the protein comprises:
a first protein comprising in any order a first domain comprising an amino acid sequence having at least 70% sequence identity to the amino acid sequence of SEQ ID NO:5, a second domain comprising an amino acid sequence having at least 70% sequence identity to the amino acid sequence of SEQ ID NO:6, a third domain comprising an amino acid sequence having at least 70% sequence identity to the amino acid sequence of SEQ ID NO:7, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a second protein comprising a first domain comprising an amino acid sequence having at least 70% sequence identity to the amino acid sequence of SEQ ID NO:25;
a third protein comprising in any order a first domain comprising an amino acid sequence having at least 70% sequence identity to the amino acid sequence of SEQ ID NO:1, a second domain comprising an amino acid sequence having at least 70% sequence identity to the amino acid sequence of SEQ ID NO:5, a third domain comprising an amino acid sequence having at least 70% sequence identity to the amino acid sequence of SEQ ID NO:6, and a fourth domain comprising an amino acid sequence having at least 70% sequence identity to the amino acid sequence of SEQ ID NO:10;
a fourth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:2;
a fifth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:5;
a sixth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:6;
a seventh protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:5, and a fourth domain comprising an amino acid sequence having at least 70% sequence identity to the amino acid sequence of SEQ ID NO:6;
an eighth protein comprising in any order a first domain comprising an amino acid sequence having at least 70% sequence identity to the amino acid sequence of SEQ ID NO:3, and a second domain comprising an amino acid sequence having at least 70% sequence identity to the amino acid sequence of SEQ ID NO:7;
a ninth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:8, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a tenth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:10; or
an eleventh protein comprising in any order a first domain comprising an amino acid sequence having at least 70% sequence identity to the amino acid sequence of SEQ ID NO:25, a second domain comprising an amino acid sequence having at least 70% sequence identity to the amino acid sequence of SEQ ID NO:8, a third domain comprising an amino acid sequence having at least 70% sequence identity to the amino acid sequence of SEQ ID NO:9, and a fourth domain comprising an amino acid sequence having at least 70% sequence identity to the amino acid sequence of SEQ ID NO:10.

Aspect 2. A polynucleotide encoding a protein comprising at least two different domains and no greater than six different domains, wherein each domain is selected from an amino acid sequence having at least 70% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:25.

Aspect 3. The polynucleotide of Aspect 1 or 2, wherein the polynucleotide sequence is present in a vector.

Aspect 4. The polynucleotide of any one of Aspects 1-3, wherein the vector comprises a plasmid vector or a viral vector.

Aspect 5. The polynucleotide of any one of Aspects 1-4, wherein the viral vector is an adenovirus vector, a poxvirus vector, an alphavirus vector, a retrovirus vector, a vaccinia virus vector, or a lentivirus vector.

Aspect 6. The polynucleotide of any one of Aspects 1-5, wherein the adenovirus vector is a replication defective adenovirus vector.

Aspect 7. The polynucleotide of any one of Aspects 1-6, wherein the replication defective adenovirus vector is type-5 (Ad5).

Aspect 8. The polynucleotide of any one of Aspects 1-7, wherein the polynucleotide is a mRNA.

Aspect 9. The polynucleotide of any one of Aspects 1-8, wherein the mRNA is complexed with a lipid carrier.

Aspect 10. The polynucleotide of any one of Aspects 1-9, wherein the mRNA comprises a 5′ cap structure and a 3′ region.

Aspect 11. A genetically modified cell comprising the polynucleotide sequence of any one of Aspects 1-10.

Aspect 12. A viral particle comprising the polynucleotide of any one of Aspects 1-10.

Aspect 13. The viral particle of Aspect 12, wherein the viral particle is an adenovirus viral particle.

Aspect 14. A protein, wherein the protein comprises:
a first protein comprising in any order a first domain comprising an amino acid sequence having at least 70% sequence identity to the amino acid sequence of SEQ ID NO:5, a second domain comprising an amino acid sequence having at least 70% sequence identity to the amino acid sequence of SEQ ID NO:6, a third domain comprising an amino acid sequence having at least 70% sequence identity to the amino acid sequence of SEQ ID NO:7, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a second protein comprising a first domain comprising an amino acid sequence having at least 70% sequence identity to the amino acid sequence of SEQ ID NO:25;
a third protein comprising in any order a first domain comprising an amino acid sequence having at least 70% sequence identity to the amino acid sequence of SEQ ID NO:1, a second domain comprising an amino acid sequence having at least 70% sequence identity to the amino acid sequence of SEQ ID NO:5, a third domain comprising an amino acid sequence having at least 70% sequence identity to the amino acid sequence of SEQ ID NO:6, and a fourth domain comprising an amino acid sequence having at least 70% sequence identity to the amino acid sequence of SEQ ID NO:10;
a fourth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:2;
a fifth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:5;
a sixth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:6;
a seventh protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:5, and a fourth domain comprising an amino acid sequence having at least 70% sequence identity to the amino acid sequence of SEQ ID NO:6;
an eighth protein comprising in any order a first domain comprising an amino acid sequence having at least 70% sequence identity to the amino acid sequence of SEQ ID NO:3, and a second domain comprising an amino acid sequence having at least 70% sequence identity to the amino acid sequence of SEQ ID NO:7;
a ninth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:8, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a tenth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:10; or
an eleventh protein comprising in any order a first domain comprising an amino acid sequence having at least 70% sequence identity to the amino acid sequence of SEQ ID NO:25, a second domain comprising an amino acid sequence having at least 70% sequence identity to the amino acid sequence of SEQ ID NO:8, a third domain comprising an amino acid sequence having at least 70% sequence identity to the amino acid sequence of SEQ ID NO:9, and a fourth domain comprising an amino acid sequence having at least 70% sequence identity to the amino acid sequence of SEQ ID NO:10.

Aspect 15. A protein comprising at least two different domains and no greater than six different domains, wherein each domain is selected from an amino acid sequence having at least 70% identity to SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and SEQ ID NO:25.

Aspect 16. The protein of any one of Aspects 14-15, wherein the protein further comprises a linker between the first and second domains of the fourth, eighth, or tenth protein.

Aspect 17. The protein of any one of Aspects 14-16, wherein the protein further comprises a linker between at least 2 domains of the first, third, fifth, sixth, seventh, ninth, or eleventh protein.

Aspect 18. The protein of any one of Aspects 14-17, wherein at least one linker comprises one or more glycine residues.

Aspect 19. The protein of any one of Aspects 14-18, wherein at least one linker comprises the amino acid sequence GGGGSGGGGSGGGGS (SEQ ID NO:33).

Aspect 20. The protein of any one of Aspects 14-19, further comprising a protein encoded by a SARS-CoV-2 genome.

Aspect 21. A composition comprising the protein of any one of Aspects 14-20.

Aspect 22. The composition of Aspect 21, further comprising a pharmaceutically acceptable carrier.

Aspect 23. The composition of Aspect 22, further comprising an adjuvant.

Aspect 24. A composition comprising the polynucleotide of any one of Aspects 1-10.

Aspect 25. The composition of Aspect 24, further comprising a pharmaceutically acceptable carrier.

Aspect 26. The composition of Aspect 25 further comprising an adjuvant.

Aspect 27. A method comprising:
administering to a subject an amount of the composition of any one of Aspects 21-26 effective to induce an immune response in the subject, wherein the immune response comprises (i) antibody, (ii) helper T cells, (iii) suppressor T cells, and/or (iv) cytotoxic T cells, directed to an epitope of a protein present in the composition or encoded by the polynucleotide.

Aspect 28. The method of Aspect 27, wherein a single dose of the composition is administered.

Aspect 29. The method of any one of Aspects 27-28, wherein a first dose of the composition is administered and a second dose is administered as an additional administration.

Aspect 30. The method of any one of Aspects 27-29, wherein a first dose and a second dose are administered at the same time by different or similar routes.

Aspect 31. The method of any one of Aspects 27-30, wherein the first dose and the second dose are the same composition.

Aspect 32. The method of any one of Aspects 27-31, wherein the first dose and the second dose are different compositions.

Aspect 33. The method of any one of Aspects 27-32, wherein a first dose is administered and a second dose administered at least one week later.

Aspect 34. The method of any one of Aspects 27-33, wherein the first dose and the second dose are the same composition.

Aspect 35. The method of any one of Aspects 27-34, wherein the first dose and the second dose are different compositions.

Aspect 36. The method of Aspect any one of Aspects 27-35, wherein the subject has a pre-existing immune response to a SARS-CoV-2 and the second dose comprises the composition.

Aspect 37. The method of any one of Aspects 27-36, wherein the pre-existing immune response is the result of prior immunization.

Aspect 38. The method of any one of Aspects 27-37, wherein the prior immunization comprises immunization with a vaccine comprising an mRNA or a DNA.

Aspect 39. A method for treating an infection in a subject, the method comprising:
administering an effective amount of the composition of any one of Aspects 21-26 to a subject at risk of having an infection caused by a coronavirus.

Aspect 40. A method for treating a sign of infection in a subject, the method comprising:
administering an effective amount of the composition of any one of Aspects 21-26 to a subject having or at risk of having an infection caused by a coronavirus.

Aspect 41. A method for treating a condition in a subject, the method comprising:
administering an effective amount of the composition of any one of Aspects 21-26 to a subject in need thereof, wherein the subject has or is at risk of having a condition caused by a coronavirus.

Aspect 42. The method of any one of Aspects 39-41, wherein the coronavirus is SARS-CoV-2.

Aspect 43. The method of any one of Aspects 27-, wherein the administering comprises a topical administration or an intramuscular administration.

Aspect 44. The method of any one of Aspects 27-43, wherein the topical administration comprises delivery to the nasal or respiratory mucosa, or a combination thereof.

Aspect 45. The method of any one of Aspects 27-44, wherein the method further comprises at least one additional primary administration.

Aspect 46. The method of any one of Aspects 27-45, wherein the method further comprises at least one booster administration.

Aspect 47. The method of any one of Aspects 27-46, wherein the booster administration comprises a topical administration or an intramuscular administration.

Aspect 48. The method of any one of Aspects 27-47, wherein the booster administration comprises delivery to the nasal or respiratory mucosa, or a combination thereof.

Aspect 49. The method of any one of Aspects 27-48, wherein the subject is a mammal.

Aspect 50. The method of any one of Aspects 27-49, wherein the mammal is a human.

Aspect 51. The method of any one of Aspects 27-50, wherein the mammal is a mouse, hamster, ferret, or non-human primate.

Aspect 52. The method of any one of Aspects 27-51, wherein the composition administered comprises (i) more than one of the polynucleotides, wherein each polynucleotide encodes a different protein, or (ii) more than one of the proteins.

Aspect 53. The method of any one of Aspects 27-52, wherein the composition comprises 2 different polynucleotides, 3 different polynucleotides, 4 different polynucleotides, 5 different polynucleotides, 6 different polynucleotides, 7 different polynucleotides, 8 different polynucleotides, 9 different polynucleotides, or 10 different polynucleotides.

Aspect 54. The method of any one of Aspects 27-53, wherein the composition comprises 2 different proteins, 3 different proteins, 4 different proteins, 5 different proteins, 6 different proteins, 7 different proteins, 8 different proteins, 9 different proteins, or 10 different proteins.

Aspect 55. The method of any one of Aspects 27-54, wherein the administering comprises separate administration of two or more compositions, wherein each composition comprises (i) a different polynucleotide, or (ii) a different protein.

Aspect 56. The method of any one of Aspects 27-55, wherein the administration comprises separate administration of two compositions, three compositions, four compositions, five compositions, six compositions, seven compositions, eight compositions, nine compositions, or ten compositions, wherein each composition comprises a different polynucleotide.

Aspect 57. The method of any one of Aspects 27-56, wherein the administration comprises separate administration of two different proteins, three different proteins, four different proteins, five different proteins, six different proteins, seven different proteins, eight different proteins, nine different proteins, or ten different proteins.

Aspect 58. The method of any one of Aspects 27-57, wherein at least one additional primary administration comprises (i) a polynucleotide that encodes a protein that is different than the protein of first administration, or (ii) a protein that is different than the protein of the first administration.

Aspect 59. The method of any one of Aspects 27-58, wherein at least one booster administration comprises (i) a polynucleotide that encodes a protein that is different than the protein of first administration, or (ii) a protein that is different than the protein of the first administration.

Aspect 60. The method of any one of Aspects 27-59, wherein at least one additional primary administration comprises administration of a composition comprising 2 different polynucleotides, 3 different polynucleotides, 4 different polynucleotides, 5 different polynucleotides, 6 different polynucleotides, 7 different polynucleotides, 8 different polynucleotides, 9 different polynucleotides, or 10 different polynucleotides.

Aspect 61. The method of any one of Aspects 27-60, wherein at least one booster comprises administration of a composition comprising 2 different polynucleotides, 3 different polynucleotides, 4 different polynucleotides, 5 different polynucleotides, 6 different polynucleotides, 7 different polynucleotides, 8 different polynucleotides, 9 different polynucleotides, or 10 different polynucleotides.

Aspect 62. The method of any one of Aspects 27-61, wherein at least one additional primary administration comprises administration of a composition comprising 2 different proteins, 3 different proteins, 4 different proteins, 5 different proteins, 6 different proteins, 7 different proteins, 8 different proteins, 9 different proteins, or 10 different proteins.

Aspect 63. The method of any one of Aspects 27-62, wherein at least one booster comprises administration of a composition comprising 2 different proteins, 3 different proteins, 4 different proteins, 5 different proteins, 6 different proteins, 7 different proteins, 8 different proteins, 9 different proteins, or 10 different proteins.

Aspect 64. An isolated antibody-producing cell, helper T cell, suppressor T cell, or cytotoxic T cell that is stimulated by an epitope of a protein of any one of Aspects 14-20.

Aspect 65. The method of any one of Aspects 27-63, further comprising a step of isolating from the subject:
an antibody that specifically binds to an epitope of one of the proteins;
a nucleic acid encoding an antibody that specifically binds to an epitope of one of the proteins; or
a cell comprising a nucleic acid sequence encoding an antibody that specifically binds to an epitope of one of the proteins; or
an immune protective component, with therapeutic effectiveness or specificity against coronavirus, that result from the administering, that may be isolated, identified, modified, and/or used to diagnose, treat, or prevent coronavirus infections.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Vaccines for the Treatment of COVID (Corona Virus Disease)-19 Caused by Severe Acute Respiratory Syndrome Coronavirus (SARS-CoV)-2; an etiological agent of the current pandemic

Introduction and Vaccine Design

Coronaviruses (CoVs) are enveloped, positive-sense, and single-stranded RNA viruses and belong to the subfamily Coronavirinae, family Coronavirdiae, and order Nidovirales. There are four genera of CoVs, namely Alphacoronavirus (αCoV), Betacoronavirus (βCoV), Deltacoronavirus (δCoV), and Gammacoronavirus (γCoV). HCoV-229E and HKU-NL63 are αCoVs, while HCoV-OC43, HCoV-HKU1, SARS-CoV, MERS-CoV, and SARS-CoV-2 belong to βCoVs (1, 5).

Structure of SARS-CoV-2 and functionality of various proteins: The SARS-CoV-2 genome comprises of 29,891 nucleotides, which encode 12 open reading frames (ORFs) responsible for the synthesis of viral structural and nonstructural proteins (2). A mature SARS-CoV-2 has four structural proteins (Sps), namely envelope (E), membrane (M), nucleocapsid (N), and spike (S). All these proteins serve as antigens to stimulate neutralizing antibodies against the virus and to trigger CD4+/CD8+ T-cell responses (2, 6, 7). The S protein consists of S1 (685 amino acid [aa] residues) and S2 (588 aa) regions, with the S1 encompassing the receptor (Angiotensin-Converting Enzyme [ACE]-2)-binding domain (RBD), and S2 allowing virus entry into the host cell (1, 2). The S1-spike protein of SARS-CoV-2 shares ˜70% and 20% identity, respectively, with that of human SARS-CoV and MERS-CoV (1, 2). The highly variable region within the S1-spike protein is the RBD subdomain, while, the S2 region is relatively conserved across CoVs (1, 2). Within S2, there are two heptad repeat domains (HR1 and HR2), and antibodies to HR2 have strong virus-neutralizing activity (8, 9). The E and M proteins function in viral assembly, while the N protein is essential for viral RNA synthesis (2, 10). The M protein augments N protein induced immune responses (2, 11). Although the N protein is more conserved among SARS-CoV-2, SARS-CoV, and MERS-CoV, low sequence similarity is noted among the other four human coronaviruses that lead to mild symptoms in humans (12).

In addition to the above structural proteins, some non-structural proteins (Nsps), especially Nsp3 and Nsp8, are predicted to be adhesins, based on in silico Vaxign reverse vaccinology programs, which are crucial to the viral adhering and host invasion (12). Importantly, Nsp3 and Nsp8 also contain promiscuous major histocompatibility complex (MHC)-I and MHC-II T-cell epitopes as well as linear B-cell epitopes, localized in specific locations and functional domains of the proteins (12). Nsp3 is the largest protein encoded by the CoV genomes, with an average molecular mass of about 200 kDa (13).

Nsp3 is an essential component of the replication/transcription complex (13). It is comprised of various domains, the organization of which differs among CoV genera, due to duplication or absence of some domains (13). However, eight domains of Nsp3 exist in all known CoVs and include: 1) the ubiquitin-like domain 1 (Ubl1), 2) the Glu-rich acidic domain, 3) a macrodomain, 4) the ubiquitin-like domain 2 (Ubl2), 5) the papain-like protease 2 (PL2pro) domain, 6) the Nsp3 ectodomain (3Ecto, also called “zinc-finger domain”), as well as the domains Y1 (7) and CoV-Y (8) of unknown functions (13). In addition, the two transmembrane regions, TM1 and TM2, exist in all CoVs (13). Nsp3 was found to be more conserved among SARS-CoV-2, SARS-CoV, and MERS-CoV than other coronaviruses infecting human and other animals (12). The Ubl1 is located at the N-terminus of Nsp3 and functions in single stranded (ss) RNA binding and interacting with the nucleocapsid (N) protein (13-16). It seems to be important for virus replication as well as in initiating viral infection (13). Nsp3 of CoVs is thought to pass the endoplasmic reticulum (ER) membrane twice, and the 3Ecto of Nsp3 is the only domain located on the luminal side of the ER (13). The transmembrane regions plus the 3Ecto are important for the PL2pro to process the Nsp3↓4 cleavage site in SARS-CoV (13, 17). It has been shown that interaction of the 3Ecto with the luminal loop of Nsp4 is essential for the ER rearrangements occurring in cells infected with the SARS-CoV (13, 18).

The replication of SARS-CoV genome is believed to involve two RNA-dependent RNA polymerase (RdRp) activities, which is unique among RNA viruses (19). The first is primer-dependent and associated with a 106-kDa non-structural protein 12 (Nsp12), whereas the second is catalyzed by a 22-kDa Nsp8 (19). This latter enzyme is capable of de novo initiation and has been proposed to operate as a primase (19). Interestingly, this protein has only been crystallized together with a 10-kDa Nsp7, forming a hexadecameric, dsRNA-encircling ring structure [i.e., Nsp(7+8), consisting of 8 copies of both Nsps]. The Nsp8's N-terminus is critical for both the protein's ability to associate with Nsp7 and to boost its RdRp activity (19). We have described above-mentioned antigens as they play pivotal roles in SARS-CoV-2 virulence and in designing effective vaccines.

Vaccines for COVID-19: Four vaccines have been approved for Emergency Use by the FDA or have sought full FDA approval; Pfizer-BioNTech COVID-19 vaccine, Moderna COVID-19 vaccine, Astra Zeneca COVID-19 vaccine, and Johnson & Johnson COVID-19 vaccine, and an antiviral remdesivir has shown some promise and approved by FDA for emergency purposes in a hospital setting (20, 21). In view of the surging infections due to the Delta variant continued development of SARS-CoV-2-based vaccines is urgently required, and currently, number of SARS-CoV-2 vaccine candidates are under development and include: 1) the inactivated or attenuated virus particle-based vaccines, 2) the virus protein-based subunit vaccines, 3) DNA or mRNA vaccines, and 4) the viral vector-based vaccines (https://www.who.int/who-documents-detail/draft-landscape-of-covid-19-candidate-vaccines). These vaccine candidates are in different developmental stages and some of them are in expedited phase III clinic trials. Based on the previous experience with SARS and MERS-CoVs (2, 12, 24, 25), the S-based vaccines have some drawbacks related to the lack in inducing complete protection and safety concerns (e.g., lung pathology).

Vaccines: We have used replication-defective Human Adenovirus 5 (Ad5) as a viral vector to express fusion genes that encode both structural proteins (S, M, and N) and non-structural proteins (Nsp3 and Nsp8) from SARS-CoV-2 or MERS-CoV in various combinations. The “Sp/Nsp cocktail vaccine(s)” containing both structural protein(s) (Sps) and a non-structural protein(s) (Nsps) would stimulate effective complementary immune responses to combat all human CoVs. Our multicomponent Ad5-based vaccines would include: 1) S1 and MERS S1-RBD (912 aa); 2) S1-RBD, S2-HR2 and M protein (669 aa); 3) S1-RBD, S2-HR2, and N protein (419 aa); 4) S1-RBD, S2-HR2, M and N proteins (1103 aa); 5) S1-RBD and Nsp3 (2217aa); 6) S1-RBD, S2-HR2, Ubl1-Nsp3, 3Ecto-Nsp3, and Nsp8 (872 aa); 7) S1-Spike protein and Nsp8 (902 aa); 8) combinations of these multi-components with full Spike protein.

Generation of the constructs: To make the above constructs, the components in each fusion gene were interconnected via a small DNA fragment that encoded a 15 amino acid flexible linker (GGGGSGGGGSGGGGS (SEQ ID NO:33)). The fusion gene cassettes were codon optimized for expression in humans, which also allowed us to optimize secondary structures of the corresponding RNAs and removal of unwanted sites for the restriction enzymes, except for those used for cloning purposes. To improve expression of the corresponding fusion genes, the Kozak consensus sequence was also placed upstream of the start codon. The constructs were then synthesized and verified via DNA sequence analysis. Each verified synthetic construct was cloned into the pShuttleX vector under the control of a cytomegalovirus (CMV) promoter.

To generate recombinant adenoviruses, the above fusion gene constructs with their CMV promoters were removed from the pShuttleX vector and cloned into the replication-defective human type 5 adenovirus plasmid vector Adeno-X. The resulting recombinant plasmid vectors were then linearized by the PacI restriction enzyme digestion and transfected separately into human embryonic kidney 293 (HEK293) cells. The formation of recombinant adenovirus (rAd5) plaque was monitored, and now rAd5 will be purified. We then will examine expression of the target-protein-encoding genes in A549 human lung epithelial cells that have been infected with the rAd5 at 1,000 viral particles/cell. The host cell lysates will be harvested after 24 h post infection, resolved by SDS-PAGE, and subjected to Western blot analysis with specific viral antigen antibodies. Ad5-CMV-Empty vector will serve as a control. These methods are well described in our publication (26).

Animal immunizations: Animals (mice, hamsters, ferrets, or non-human primates [NHPs]) will be immunized via the intranasal route once on day 0 or 2 doses at 21 days apart. In ferrets and NHPs, vaccination will occur via an aerosol mist. The number of rAd5 viral particles inhaled will range from 1×10⁹-1×10¹¹. Other routes of vaccination, such as intramuscular route will also be tested. In some cases, the vaccines will be thermostabilized and tested orally. At various time intervals, antibody responses, neutralizing antibody titers, as well as T cell responses will be determined to determine correlates of protection. The model systems used are commonly accepted for the study of preventing infection by coronavirus. Examples of the model systems include non-human primates, mice, hamsters, ferrets and rabbits (Chan et al., Clin Infect Dis, (2020); Bao et al., Nature, (2020); Yuan et al., Emerg Microbes Infect 9, 949-961 (2020)).

### CITATIONS FOR EXAMPLE 1

1. J. F. Chan et al., Genomic characterization of the 2019 novel human-pathogenic coronavirus isolated from a patient with atypical pneumonia after visiting Wuhan. Emerg Microbes Infect 9, 221-236 (2020).
2. W. Shang, Y. Yang, Y. Rao, X. Rao, The outbreak of SARS-CoV-2 pneumonia calls for viral vaccines. NPJ Vaccines 5, 18 (2020).
3. J. S. Peiris et al., Coronavirus as a possible cause of severe acute respiratory syndrome. Lancet 361, 1319-1325 (2003).
4. M. L. Yeung et al., MERS coronavirus induces apoptosis in kidney and lung by upregulating Smad7 and FGF2. Nat Microbiol 1, 16004 (2016).
5. J. F. Chan, K. K. To, H. Tse, D. Y. Jin, K. Y. Yuen, Interspecies transmission and emergence of novel viruses: lessons from bats and birds. Trends Microbiol 21, 544-555 (2013).
6. F. Wu et al., A new coronavirus associated with human respiratory disease in China. Nature 579, 265-269 (2020).
7. C. Huang et al., Clinical features of patients infected with 2019 novel coronavirus in Wuhan, China. Lancet 395, 497-506 (2020).
8. S. Xia et al., Inhibition of SARS-CoV-2 (previously 2019-nCoV) infection by a highly potent pan-coronavirus fusion inhibitor targeting its spike protein that harbors a high capacity to mediate membrane fusion. Cell Res 30, 343-355 (2020).
9. C. T. Keng et al., Amino acids 1055 to 1192 in the S2 region of severe acute respiratory syndrome coronavirus S protein induce neutralizing antibodies: implications for the development of vaccines and antiviral agents. J Virol 79, 3289-3296 (2005).
10. D. Schoeman, B. C. Fielding, Coronavirus envelope protein: current knowledge. Virol J 16, 69 (2019).
11. S. Q. Shi et al., The expression of membrane protein augments the specific responses induced by SARS-CoV nucleocapsid DNA immunization. Mol Immunol 43, 1791-1798 (2006).
12. E. Ong, U. M. Wong, A. Huffman, Y. He, COVID-19 coronavirus vaccine design using reverse vaccinology and machine learning. bioRxiv, (2020).
13. J. Lei, Y. Kusov, R. Hilgenfeld, Nsp3 of coronaviruses: Structures and functions of a large multi-domain protein. Antiviral Res 149, 58-74 (2018).
14. P. Serrano et al., Nuclear magnetic resonance structure of the N-terminal domain of nonstructural protein 3 from the severe acute respiratory syndrome coronavirus. J Virol 81, 12049-12060 (2007).
15. K. R. Hurst, R. Ye, S. J. Goebel, P. Jayaraman, P. S. Masters, An interaction between the nucleocapsid protein and a component of the replicase-transcriptase complex is crucial for the infectivity of coronavirus genomic RNA. J Virol 84, 10276-10288 (2010).
16. K. R. Hurst, C. A. Koetzner, P. S. Masters, Characterization of a critical interaction between the coronavirus nucleocapsid protein and nonstructural protein 3 of the viral replicase-transcriptase complex. J Virol 87, 9159-9172 (2013).
17. B. H. Harcourt et al., Identification of severe acute respiratory syndrome coronavirus replicase products and characterization of papain-like protease activity. J Virol 78, 13600-13612 (2004).
18. M. C. Hagemeijer et al., Membrane rearrangements mediated by coronavirus nonstructural proteins 3 and 4. Virology 458-459, 125-135 (2014).
19. A. J. te Velthuis, S. H. van den Worm, E. J. Snijder, The SARS-coronavirus nsp7+nsp8 complex is a unique multimeric RNA polymerase capable of both de novo initiation and primer extension. Nucleic Acids Res 40, 1737-1747 (2012).
20. J. Grein et al., Compassionate Use of Remdesivir for Patients with Severe Covid-19. N Engl J Med, (2020).
21. M. L. Agostini et al., Coronavirus Susceptibility to the Antiviral Remdesivir (GS-5734) Is Mediated by the Viral Polymerase and the Proofreading Exoribonuclease. mBio 9, (2018).
22. F. C. Zhu et al., Safety, tolerability, and immunogenicity of a recombinant adenovirus type-5 vectored COVID-19 vaccine: a dose-escalation, open-label, non-randomised, first-in-human trial. Lancet, (2020).
23. T. R. F. Smith et al., Immunogenicity of a DNA vaccine candidate for COVID-19. Nat Commun 11, 2601 (2020).
24. E. de Wit, N. van Doremalen, D. Falzarano, V. J. Munster, SARS and MERS: recent insights into emerging coronaviruses. Nat Rev Microbiol 14, 523-534 (2016).
25. R. L. Roper, K. E. Rehm, SARS vaccines: where are we? Expert Rev Vaccines 8, 887-898 (2009).
26. J. Sha et al., A Replication-Defective Human Type 5 Adenovirus-Based Trivalent Vaccine Confers Complete Protection against Plague in Mice and Nonhuman Primates. Clin Vaccine Immunol 23, 586-600 (2016).
27. J. F. Chan et al., Simulation of the clinical and pathological manifestations of Coronavirus Disease 2019 (COVID-19) in golden Syrian hamster model: implications for disease pathogenesis and transmissibility. Clin Infect Dis, (2020).
28. L. Bao et al., The pathogenicity of SARS-CoV-2 in hACE2 transgenic mice. Nature, (2020).
29. L. Yuan, Q. Tang, T. Cheng, N. Xia, Animal models for emerging coronavirus: progress and new insights. Emerg Microbes Infect 9, 949-961 (2020).

### Example 2

Evaluation of the Expression of the SARS-CoV-2 Fusion Genes in Representative Ad5 Constructs by Western Blot Analysis.

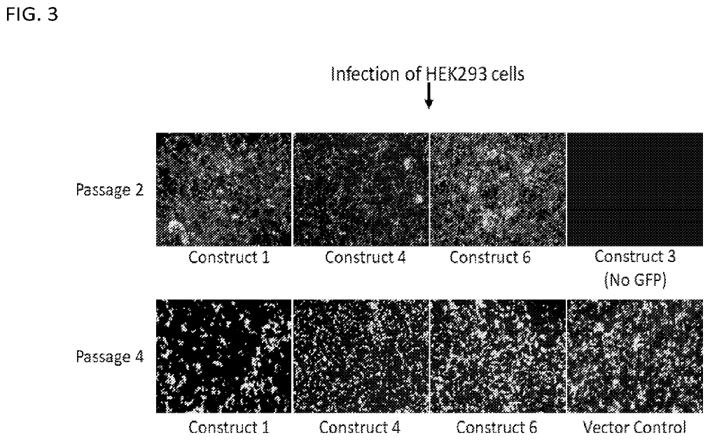

Table 1 shows Ad5 constructs described in Example 1 with their respective compositions (with SARS-CoV-2 antigens), number of amino acid residues and sizes, as well as whether or not these constructs harbored GFP (green florescent protein)-encoding gene for easy visulaization of its expression using fluorescence microscopy. As noted in FIG. 3, when construct #s 1,4, and 6 with the gfp gene were used to infect human embryonic kidney 293 (HEK293) cells, which support replication of Ad5, fluorescence could be seen in these cells. Similarly, the control Ad5 vector with the gfp gene was positive for green fluorescence, while the construct #3 without the gfp gene did not exhibit any fluorescence. For these studies, HEK293 cells were grown in DMEM+10% FBS to 80-100% confluency. Cells (in 6-well tissue culture plates) were then infected with 10 μL of purified virus (titer 1×10¹²) and incubated at 37° C.+5% C02 for 30 minutes. Medium was then aspirated and fresh medium added. GFP expression was measured after 24-48 hours until HEK293 cells began to detach. Subsequently, 50 μL of this original culture supernatant was added to fresh HEK293 cells grown to 80-100% confluency. Cells were then passed in this manner four times to observe expression of the gfp gene. Data for two serial passages of HEK293 cells (2^{nd} and 4^{th}) is shown (FIG. 3).

TABLE 1

| Construct | | # of Amino | Size |
|---|---|---|---|
| Number | Antigens | Acid residues | (kDa) |
| Vector control | Vector only + GFP | — | — |
| 1 | S1-Spike, MERS RBD | 912 | 100 |
| 2 | S1-RBD, S2-HR2, M | 669 | 74 |
| 3 | S1-RBD, S2-HR2, N | 419 | 46 |
| 4 | S1-RBD, S2-HR2, M, N | 1103 | 121 |
| 5 | S1-RBD, nsp3 | 2217 | 244 |
| 6 | S1-RBD, S2-HR2, Ubl 1-nsp3, | 872 | 96 |
| | 3Ecto-nsp3, nsp8 | | |
| 7 | S1-Spike, nsp8 | 902 | 100 |

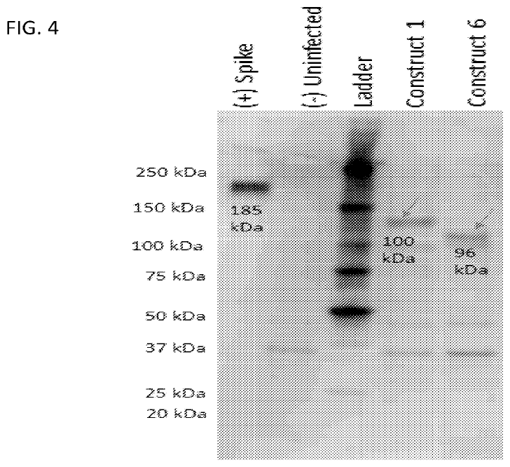

As proof-of-concept, 549 cells (adenocarcinomic human alveolar basal epithelial cells) were infected with 1000 virus particles/host cell in 6-well tissue culture plates. The A549 cells do not support replication of the virus; however, the transgenes would be expressed and can be detected by Western blot analysis using specific antibodies to the antigens. The whole cell lysates of infected A549 cells after 72 h post infection were lysed and subjected to 4-20% gradient SDS-PAGE (FIG. 4). This was followed by the transfer of proteins from the gel to the PVDF (polyvinylidene difluoride) membrane for Western blot analysis. The membrane was then probed with specific polyclonal antibodies to the spike protein. As noted from this figure, purified S protein (185 kDa) could be detected and used as a positive control. Uninfected cells served as a negative control, and construct #s 1 and 6 exhibited specific bands of 100 kDa and 96 kDa, which were the predicted sizes of the SARS-CoV-2 fusion proteins (Table 1).

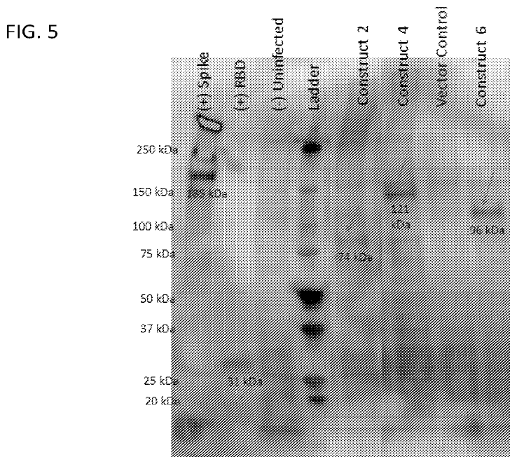

We also infected HEK293 cells with virus particles/host cell in 24-well tissue culture plates. As indicated earlier, HEK293 cells do support replication, the transgenes would be expressed, which then could be detected by Western blot analysis using specific antibodies to the antigens. The whole cell lysates of infected HEK293 cells after 24-48 h post infection were lysed and subjected to 4-20% gradient SDS-PAGE (FIG. 5). This was followed by the transfer of proteins from the gel to the PVDF (polyvinylidene difluoride) membrane for Western blot analysis. As noted from this figure, purified S protein (185 kDa) and its receptor binding domain (RBD, 31 kDa) could be detected and used as positive controls. Uninfected cells or cells infected with Ad5 vector alone served as negative controls. Representative construct #s 2, 4, and 6 exhibited specific bands of ˜74-76 kDa, 121 kDa, and 96 kDa, which were the predicted sizes of the SARS-CoV-2 fusion proteins (Table 1).

### Example 3

Evaluation of In Vivo Protection

Mouse Immunization Six- to eight-week-old female BALB/c mice (The Jackson Laboratory) were randomly grouped (5 mice per group) and allowed to acclimate for 7 days. The Ad5 vaccine candidates were administered by either the intranasal route (2 doses, 21 days apart) or one dose by the intranasal route and the second dose by the intramuscular route. Animals were immunized with 1×10¹⁰ virus particles for each dose. Negative control mice received the same volume of Ad5 vector alone. Blood was drawn from each animal on days 0 (pre-bleed), 21, and 42, and the sera were stored at −80° C.

The recommendations of the National Institutes of Health about mouse study (the Guide for the Care and Use of Laboratory Animals) were followed. All mouse experiments were approved by the Institutional Animal Care and Use Committee of the University of Texas Medical Branch (Galveston, TX). The SARS-CoV-2 virus challenge study was conducted in the animal biosafety level 3 (ABSL-3) suite in the Galveston National Laboratory (GNL).

Challenge of the mice with mouse-adapted live BSL-3 SARS-CoV-2 virus. Immunized mice were challenged with the mouse-adapted (MA) SARS-CoV-2/MA10 strain by the intranasal route. Briefly, mice were inoculated with 60 μl of SARS-CoV2-MA10 at a dose of ˜10⁵ TCID₅₀. The animals were weighed every day over the indicated period of time for monitoring the onset of morbidity (weight loss and other signs of illness) and mortality, as the endpoints for evaluating the vaccine efficacy.

In the first study, mice were immunized with Ad5 construct #s 1 and 6 (Table 1), with the first dose of the vaccine delivered by the intranasal route and the second dose by the intramuscular route on days 0 and 21, respectively.

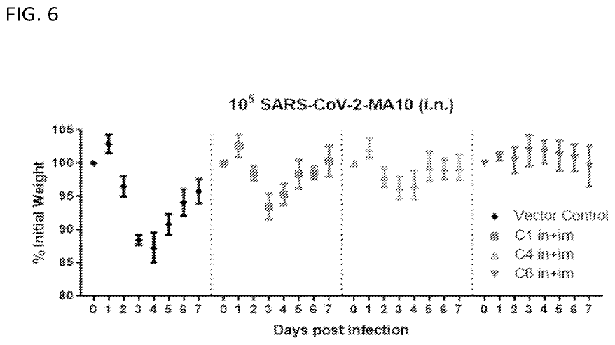

As noted from FIG. 6, animals immunized with the Ad5 vector alone, lost up to 15% of the body weight by day 4 and then showed steady recovery. However, animals immunized with Ad5 construct #s 1, 4, and 6 showed significantly less reduction in weight, with Ad5 construct #6 did not lose any weight. The loss in body weight is the most important single manifestation of infection with SARS-CoV-2. We subsequently observed that the titer of the Ad5 construct #1 was much lower (in the order of 1×10⁶ instead of 1×10¹⁰), and resulted in showing less protective effect in mice (FIG. 6).

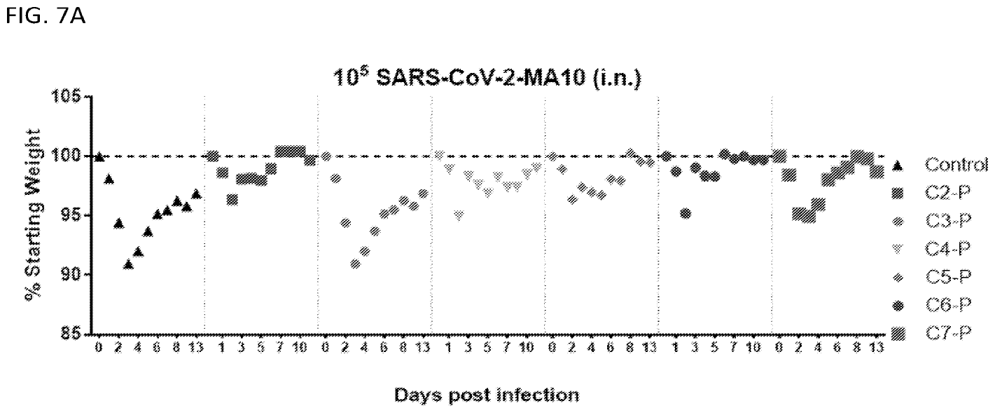

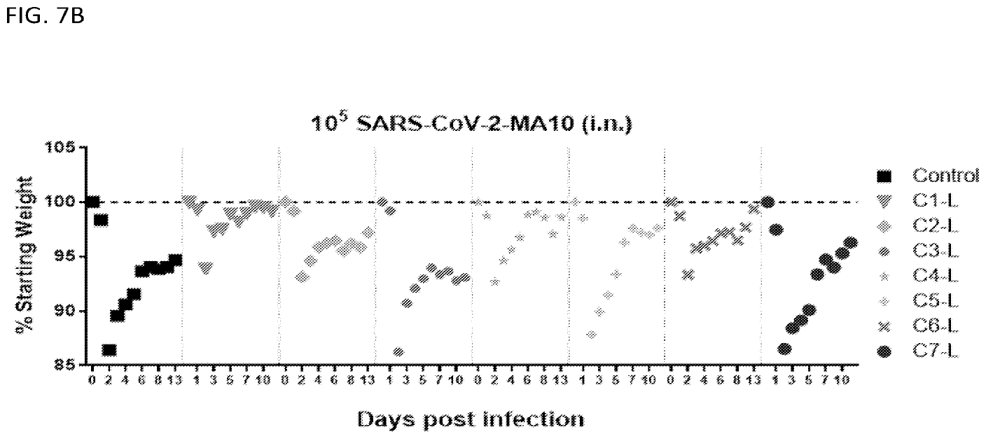

In the second experiment, mice were immunized with 2 doses of the Ad5 vaccine constructs only by the intranasal route on days 0 and 21. On days 0 and 42, animals were bled and challenged with the mouse adapted SARS-CoV-2-MA10 by the intranasal route. We used 1×10¹⁰ virus particles for immunization (indicated by the construct #s followed by the letter P-upper panel) or lower titer of the virus particles (1×10⁷, indicated by the construct #followed by the letter L-lower panel) (FIG. 7). High titer construct #1 was not available, and hence not used in the study (upper panel).

As noted from FIG. 7 (upper panel), Ad5 construct #s 2, 4, 5, 6, and 7 provided protection to mice against loss in body weight compared to Ad5 vector alone in which mice lost up to 10% of the body weight between days 2-4. While construct #2 lost minimal weight and recovered quickly from infection, construct #s 4, 5, 6, and 7 behaved similarly and were next best in terms of protective efficacy. Construct #3 did not seem to provide much protection.

When lower titer of the Ad5 constructs were used to immunize mice (FIG. 7, lower panel), Ad5 construct #s 1, 2, 4, and 6 were efficacious against infection with SARS-CoV-2 compared to animals that were vaccinated with the Ad5 vector alone. The latter group of mice lost up to 15% of the body weight by day 2. Animals immunized with Ad5 constructs #1, 2, 4, and 6 lost 5-7% body weight by day 2 but more or less fully recovered by day 7. However, Ad5 construct #s 3, 5, and 7 did not provide much protection to mice against COVID-19 at lower Ad5 dose.

### Example 4

Production of Antibody by Vaccine Constructs

ELISA for evaluating IgG antibodies to Spike protein. ELISA plates were coated with 100 μl per well of 1 μg/ml of SARS-CoV-2 S, M, or N protein in coating buffer (0.05 M sodium carbonate-sodium bicarbonate (pH 9.6)). After overnight incubation at 4° C., the plates were washed with PBS+0.05% Tween 20 buffer and blocked for 1 h at room temperature with 200 μl per well of PBS-0.1% BSA buffer. Serum samples were serially diluted (2 to 3 fold) in PBS-0.1% BSA. One hundred microliters of diluted serum samples were added to each well and the plates were incubated at room temperature for 1 h. After washing three times with PBST (PBS+0.05% Tween-20), the secondary antibody was added at 1:8,000 dilution in PBS-0.1% BSA buffer (100 μl per well) using goat-anti-mouse IgG-HRP. After incubation for 1 h at room temperature and three washes with PBST buffer, plates were developed using the TMB (3,3′, 5,5′-tetramethylbenzidine) Microwell Peroxidase Substrate System. After 2-3 min, the enzymatic reaction was stopped by adding 50 μl H₂SO₄. The absorbance was read at 450 nm on a VersaMax spectrophotometer. The endpoint titer was defined as the highest reciprocal dilution of serum that gives an absorbance more than 2-fold of the mean background of the assay.

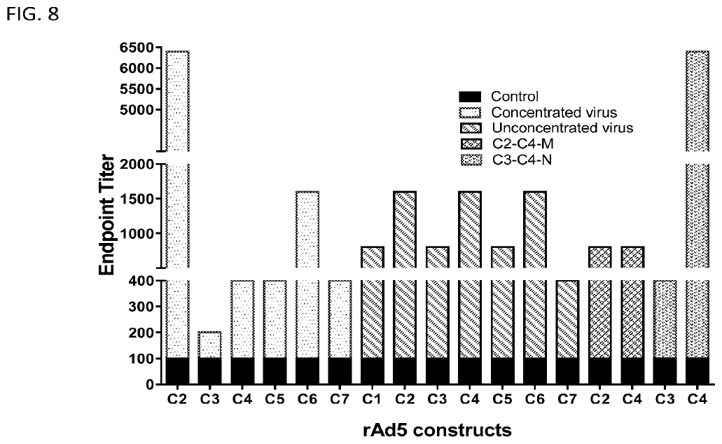

As can be noted (FIG. 8), it was possible to detect antibody titers to S, M, and N proteins with various Ad5 constructs, although their titers varied. Ad5 construct #2 generated the highest antibody titers to S protein followed by the Ad5 construct #6, while Ad5 construct #4 generated better antibody titers to the N protein compared to Ad5 construct #3. Both Ad5 construct #s 2 and 4 generated similar level of antibody titers to the M protein.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A polynucleotide encoding a protein, wherein the protein comprises:
a first protein comprising in any order a first domain comprising an amino acid sequence having at least 70% sequence identity to the amino acid sequence of SEQ ID NO:5, a second domain comprising an amino acid sequence having at least 70% sequence identity to the amino acid sequence of SEQ ID NO:6, a third domain comprising an amino acid sequence having at least 70% sequence identity to the amino acid sequence of SEQ ID NO:7, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a second protein comprising a first domain comprising an amino acid sequence having at least 70% sequence identity to the amino acid sequence of SEQ ID NO:25;
a third protein comprising in any order a first domain comprising an amino acid sequence having at least 70% sequence identity to the amino acid sequence of SEQ ID NO:1, a second domain comprising an amino acid sequence having at least 70% sequence identity to the amino acid sequence of SEQ ID NO:5, a third domain comprising an amino acid sequence having at least 70% sequence identity to the amino acid sequence of SEQ ID NO:6, and a fourth domain comprising an amino acid sequence having at least 70% sequence identity to the amino acid sequence of SEQ ID NO:10;
a fourth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:2;
a fifth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:5;
a sixth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:6;
a seventh protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:5, and a fourth domain comprising an amino acid sequence having at least 70% sequence identity to the amino acid sequence of SEQ ID NO:6;
an eighth protein comprising in any order a first domain comprising an amino acid sequence having at least 70% sequence identity to the amino acid sequence of SEQ ID NO:3, and a second domain comprising an amino acid sequence having at least 70% sequence identity to the amino acid sequence of SEQ ID NO:7;
a ninth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:8, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a tenth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:10; or
an eleventh protein comprising in any order a first domain comprising an amino acid sequence having at least 70% sequence identity to the amino acid sequence of SEQ ID NO:25, a second domain comprising an amino acid sequence having at least 70% sequence identity to the amino acid sequence of SEQ ID NO:8, a third domain comprising an amino acid sequence having at least 70% sequence identity to the amino acid sequence of SEQ ID NO:9, and a fourth domain comprising an amino acid sequence having at least 70% sequence identity to the amino acid sequence of SEQ ID NO:10.

2. (canceled)

3. The polynucleotide of claim 1, wherein the polynucleotide sequence is present in a vector.

4. The polynucleotide of claim 3, wherein the vector comprises a plasmid vector or a viral vector.

5. The polynucleotide of claim 3, wherein the viral vector is an adenovirus vector, a poxvirus vector, an alphavirus vector, a retrovirus vector, a vaccinia virus vector, or a lentivirus vector.

6. The polynucleotide of claim 5, wherein the adenovirus vector is a replication defective adenovirus vector.

7. The polynucleotide of claim 6, wherein the replication defective adenovirus vector is type-5 (Ad5).

8. The polynucleotide of claim 1, wherein the polynucleotide is a mRNA.

9. The polynucleotide of claim 8, wherein the mRNA is complexed with a lipid carrier.

10. The polynucleotide of claim 8, wherein the mRNA comprises a 5′ cap structure and a 3′ region.

11. (canceled)

12. A viral particle comprising the polynucleotide of claim 1.

13. (canceled)

14. A protein, wherein the protein comprises:
a first protein comprising in any order a first domain comprising an amino acid sequence having at least 70% sequence identity to the amino acid sequence of SEQ ID NO:5, a second domain comprising an amino acid sequence having at least 70% sequence identity to the amino acid sequence of SEQ ID NO:6 a third domain comprising an amino acid sequence having at least 70% sequence identity to the amino acid sequence of SEQ ID NO:7, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a second protein comprising a first domain comprising an amino acid sequence having at least 70% sequence identity to the amino acid sequence of SEQ ID NO:25;
a third protein comprising in any order a first domain comprising an amino acid sequence having at least 70% sequence identity to the amino acid sequence of SEQ ID NO:1, a second domain comprising an amino acid sequence having at least 70% sequence identity to the amino acid sequence of SEQ ID NO:5, a third domain comprising an amino acid sequence having at least 70% sequence identity to the amino acid sequence of SEQ ID NO:6, and a fourth domain comprising an amino acid sequence having at least 70% sequence identity to the amino acid sequence of SEQ ID NO:10;
a fourth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:2;
a fifth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:5;
a sixth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, and a third domain comprising an amino acid sequence having at least 70% sequence identity to the amino acid sequence of SEQ ID NO:6;
a seventh protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:5, and a fourth domain comprising an amino acid sequence having at least 70% sequence identity to the amino acid sequence of SEQ ID NO:6;
an eighth protein comprising in any order a first domain comprising an amino acid sequence having at least 70% sequence identity to the amino acid sequence of SEQ ID NO:3, and a second domain comprising an amino acid sequence having at least 70% sequence identity to the amino acid sequence of SEQ ID NO:7;
a ninth protein comprising in any order a first domain comprising an amino acid sequence having at least 70% sequence identity to the amino acid sequence of SEQ ID NO:3, a second domain comprising an amino acid sequence having at least 70% sequence identity to the amino acid sequence of SEQ ID NO:4, a third domain comprising an amino acid sequence having at least 70% sequence identity to the amino acid sequence of SEQ ID NO:8, a fourth domain comprising an amino acid sequence having at least 70% sequence identity to the amino acid sequence of SEQ ID NO:9, and a fifth domain comprising an amino acid sequence having at least 70% sequence identity to the amino acid sequence of SEQ ID NO:10;
a tenth protein comprising in any order a first domain comprising an amino acid sequence having at least 70% sequence identity to the amino acid sequence of SEQ ID NO:1, and a second domain comprising an amino acid sequence having at least 70% sequence identity to the amino acid sequence of SEQ ID NO:10; or
an eleventh protein comprising in any order a first domain comprising an amino acid sequence having at least 70% sequence identity to the amino acid sequence of SEQ ID NO:25, a second domain comprising an amino acid sequence having at least 70% sequence identity to the amino acid sequence of SEQ ID NO:8, a third domain comprising an amino acid sequence having at least 70% sequence identity to the amino acid sequence of SEQ ID NO:9, and a fourth domain comprising an amino acid sequence having at least 70% sequence identity to the amino acid sequence of SEQ ID NO:10.

15-19. (canceled)

20. The protein of claim 14, further comprising a protein encoded by a SARS-CoV-2 genome.

21. A composition comprising one of the proteins of claim 14.

22. The composition of claim 21, further comprising a pharmaceutically acceptable carrier and an adjuvant.

23. (canceled)

24. A composition comprising one of the polynucleotides of claim 1.

25. The composition of claim 24, further comprising a pharmaceutically acceptable carrier and an adjuvant.

26. (canceled)

27. A method comprising:
administering to a subject an amount of the composition of claim 21 effective to induce an immune response in the subject, wherein the immune response comprises (i) antibody, (ii) helper T cells, (iii) suppressor T cells, and/or (iv) cytotoxic T cells, directed to an epitope of a protein present in the composition or encoded by the polynucleotide.

28. The method of claim 27, wherein a single dose of the composition is administered.

29. The method of claim 27, wherein a first dose of the composition is administered and a second dose is administered as an additional administration.

30. The method of claim 27, wherein a first dose and a second dose are administered at the same time by different or similar routes.

31-65. (canceled)


SEQUENCE LISTING (33 sequences)

SEQ ID NO: 1 - protein, 674 aa, SARS-CoV-2
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 2 - protein, 208 aa, SARS-CoV-2 [sequence omitted]

SEQ ID NO: 3 - protein, 242 aa, SARS-CoV-2
FTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNF

SEQ ID NO: 4 - protein, 164 aa, SARS-CoV-2
YHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYI

SEQ ID NO: 5 - protein, 218 aa, SARS-CoV-2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIA

SEQ ID NO: 6 - protein, 419 aa, SARS-CoV-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 7 - protein, 1945 aa, SARS-CoV-2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 8 - protein, 111 aa, SARS-CoV-2
APTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDE

SEQ ID NO: 9 - protein, 82 aa, SARS-CoV-2
SNLGMPSYCTGYREGYLNSTNVTIATYCTGSIPCSVCLSGLDSLDTYPSLETIQITISSFKWDLTAFGLVAEWFLAYILFTR

SEQ ID NO: 10 - protein, 198 aa, SARS-CoV-2
AIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 11 - protein, 912 aa, artificial
  Fusion protein including SARS-CoV-2 S1 and MERS S1-RBD
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARGGGGSGGGGSGGGGSVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKL

SEQ ID NO: 12 - DNA, 2739 bp, artificial
  Nucleotide sequence encoding SEQ ID NO11
atgttcgtgtttttggtactgctgcctctggtgtcctcacaatgcgtaaatctcaccactagaacccaactgccacccgcttatacgaactccttcacacgaggtgtttactaccccgataaggtctttagatcatccgtactccactcaacacaggacttgttcttgccttttttcagtaatgtcacgtggtttcacgcgatacatgtgtcaggtacaaacggcacaaagcgattcgacaaccctgtgctcccctttaacgatggcgtctattttgcctctactgagaagtcaaatattatccgcgggtggatctttgggacaaccttggattcaaagactcagtctctgcttatagtaaataacgccaccaacgtcgtcatcaaggtttgtgagttccagttttgtaatgatccatttttgggtgtctactaccataaaaacaacaagtcatggatggaatccgagttcagagtttattcctcagcgaacaattgtacttttgaatatgtgagccagccgtttctgatggatctggagggaaagcagggaaacttcaagaacctgagagagttcgttttcaaaaacatcgatggctacttcaaaatctatagcaagcacacaccaattaacctggtgcgcgatctgccccaaggcttctccgcactggaacctctggtcgatctgccaattggaataaatatcacaagatttcagaccctccttgctcttcacagaagttatctgacaccaggcgatagtagctcaggctggacagcaggggcagctgcgtattatgtgggctatctgcaacccagaacctttctcctcaagtacaacgaaaacggaactatcaccgacgccgtagattgtgctcttgacccactctccgaaactaagtgtaccttgaaatcctttactgtggaaaagggcatttaccaaacctcaaattttagagttcaacccaccgaaagtattgtacgctttccgaacattacaaatttgtgccccttcggcgaagtgtttaacgccactagattcgctagttttactgtagaagtgtatgcctggaaccggaagcggattagtaactgtgtcgccgattacagcgtgctctataacagcgcttccttcagcacatttaagtgctatggggtcagtcccaccaaacttaacgacctgtgcttcaccaacgtgtatgccgacagctttgtgatacgaggcgatgaggtgagacagattgctccagggcagacaggtaagatcgccgattacaactataaactcccagacgattttaccggttgcgtgattgcttggaattccaataatttggacagcaaggtcgggggcaactataattatctgtacagactgtttcgcaagtccaacctgaagccctttgagagagacatttcaaccgagatctatcaagctggttcaacgccttgtaatggcgtcgaaggatttaactgctatttccccctccagagttacggcttccagcccaccaacggagttggataccaaccttatcgcgtagtcgtactttcctttgaactgctgcatgcccctgctactgtctgcggacccaaaaaatcaactaatctggtaaagaacaaatgcgtgaactttaatttcaatggcctgactggtaccggtgtcctcaccgaatccaacaagaaattcctgccttttcagcaattcggtcgcgatatcgctgacacaactgacgcagtgagggacccacaaacactggaaatattggacataacgccctgtagctttggcggagtgtctgtcattacccctggcacaaatacaagcaaccaggtcgccgtcctgtaccaagatgtcaattgtacagaggtgcctgtcgcaatacatgccgaccaactcacccccacctggcgagtgtacagcaccggatcaaatgtcttccagacccgcgctgggtgcctgattggagcagaacatgtcaataactcctatgaatgtgatatacccattggcgctggaatttgcgcttcttaccaaactcagaccaatagtcctcggcgggcccgcggcggaggtggttcaggcggcggcggatctggcggaggagggtcagtcgagtgtgatttctcccccctgttgtcaggtacgcctcctcaggtgtataatttcaagaggctggtgttcacaaattgcaactacaaccttacaaagctcctgagcctgttctccgtaaatgatttcacctgttctcagatctccccagcagctattgcaagcaactgttattccagcctcattctggattactttagctatcccctgagcatgaagtctgatctgtccgtttcatctgctggacctataagtcaattcaactataaacagagctttagtaaccccacatgtctgatcctcgccaccgtgcctcataatctcaccaccatcacgaaaccactgaagtatagttacattaacaagtgttccaggcttttgagtgatgatcggactgaggtaccacagcttgtcaatgccaaccagtattccccttgcgttagcatcgttccctccacagtgtgggaggacggagactactatcgcaaacagctgtcccccttggagggcggagggtggctcgtcgcttcagggtctaccgttgctatgacagagcagctgcaaatgggcttcggtatcaccgttcagtatggcacagacaccaattccgtctgccccaagctgtga

SEQ ID NO: 13 - protein, 669 aa, artificial
  Fusion protein including S1-RBD, S2-HR2 and M
MFVFLVLLPLVSSQCFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFGGGGSGGGGSGGGGSYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIGGGGSGGGGSGGGGSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIA

SEQ ID NO: 14 - DNA, 2010 bp, artificial
  Nucleotide sequence encoding SEQ ID NO13
atgtttgtgttcctggtgcttcttccgctggtctcttcccaatgtttcactgtggagaaaggcatctatcagacttcaaatttccgggttcagccaaccgagagtattgtccgattcccaaatataacaaatctgtgccctttcggagaagtatttaacgccaccagattcgcatcctttaccgttgaggtctacgcatggaatagaaaaaggatctcaaactgcgtagctgattacagcgttctgtacaactccgcatcattctcaaccttcaagtgctatggcgtcagtcccacaaagcttaacgatctctgcttcaccaatgtgtacgccgactcattcgtgattcgaggagatgaggtacggcaaattgccccgggtcaaactggtaagattgcagactataactataagctgcctgatgactttacagggtgtgtcatagcctggaattcaaacaatctggactctaaagtgggtggcaattataattatttgtatagactgttcaggaaatctaacctcaagccctttgagcgggacatcagcaccgaaatctatcaagccggatctaccccctgcaacggagtagagggcttcaattgttatttccctttgcagagctacgggttccagccaaccaatggagtgggatatcaaccatatcgcgtggtggtgcttagctttgagttgttgcacgctcctgcaactgtatgtggcccgaaaaaatcaacaaatctggtaaaaaacaaatgtgttaactttaacttcggtggaggcggcagcggtggcggcggaagtggcggcggaggtagctaccatctgatgtcattcccacagagcgctccgcacggcgtggtcttcttgcacgtgacgtacgtacccgcacaggaaaagaactttacgacagctcctgccatttgtcacgacggtaaggcccatttccccagagagggagtctttgtatcaaatggaacccactggtttgttacacaaagaaatttttatgagccacagataatcactaccgacaacactttcgtgagtggcaattgtgacgttgtgatcgggatagtaaacaataccgtctatgacccactgcagcccgaactggatagtttcaaggaagagcttgataaatacttcaaaaatcatacatcacctgatgttgatctcggagacatcagtggcatcaacgcatcagtggtaaatattcaaaaagagatcgacaggctgaatgaagttgcaaaaaatctcaatgaatctcttatcgatctgcaggagctgggaaagtatgaacagtacataggtggggggggaagtggaggcggcggctccggcggaggcggttctatggctgacagcaatggtacgatcactgtggaagagcttaaaaagctcctggagcaatggaacttggtcataggtttcctctttctgacatggatttgtttgctgcagtttgcctatgccaatcgcaatcgcttcttgtatattatcaagctcatttttttgtggcttctgtggccagtcactcttgcatgcttcgtgctcgccgccgtatatcgaattaactggattactggcggaattgccatagcgatggcgtgtctcgtcggccttatgtggctgtcctacttcattgcaagctttagactgtttgccagaaccagatccatgtggagttttaacccagagacaaacattctgcttaatgttccccttcacggaactatcctgacccggccactgttggaatccgaactcgtgattggcgcagtgatcctgcgggggcacctgagaatcgcaggtcatcatctcggaagatgcgacatcaaggatttgccgaaggaaattaccgtggccacctcaaggactctgtcttactacaagctgggagcgtcacagagggtcgccggcgactccggctttgcagcgtattctcgctacaggattggcaattacaagcttaacaccgaccacagttcatcctcagacaacattgcgtga

SEQ ID NO: 15 - protein, 870 aa, artificial
  Fusion protein including S1-RBD, S2-HR2, and N
MFVFLVLLPLVSSQCFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFGGGGSGGGGSGGGGSYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 16 - DNA, 2613 bp, artificial
  Nucleotide sequence encoding SEQ ID NO15
atgttcgtgtttctcgtgcttctccctctggtctcatctcagtgctttaccgtcgaaaagggaatctatcaaacgagtaatttccgggtgcagccaaccgaaagtatcgtaagatttcccaacatcactaacctttgcccattcggcgaagtttttaatgctactagattcgccagtttcacagtcgaagtgtatgcctggaatagaaagagaatctccaactgcgttgctgactatagcgtgctgtacaactccgcgtcattctctactttcaagtgttacggcgtatctcctaccaagctgaacgatctctgctttaccaatgtgtacgccgattcattcgtgatacgcggtgatgaggtacgccaaattgccccgggccagacaggaaagatcgctgactataactataaactcccggacgactttactggatgcgttatcgcctggaatagtaataatctggattccaaagtggggggcaactataattatttgtatcgcctgttcaggaaatccaatctgaaacctttcgagcgcgacatatcaactgagatatatcaggccggttcaaccccatgtaatggcgtcgaaggattcaactgttattttcccctgcaaagctatggttttcagccgacaaacggcgtaggataccaaccttacagagtggtggtgttgagtttcgagctcctgcacgcccctgctacagtctgcggtcccaagaagagtacaaacctggttaagaataaatgcgtcaattttaactttgggggaggtgggtctgggggtggcggctcaggggggggcggttcctaccatctgatgagctttccccagtccgcaccacatggcgtggtattcctccacgttacctacgtacccgcccaagagaagaattttactaccgccccagcaatatgtcacgacgggaaagcacatttcccaagagagggtgtttttgtgtccaatggtacacactggtttgttacgcagcgcaatttctacgaaccacaaatcataaccacagataatacattcgtgtccggaaattgtgacgtcgtaatcggtattgtcaacaacactgtgtacgatcccctgcagccagagctggatagctttaaagaagagttggacaaatattttaaaaatcacacatcacccgatgtcgacctgggagacattagtgggatcaacgcctctgtcgtaaacatccaaaaagagattgaccggctgaatgaggttgctaagaacctgaatgagagcctgattgacttgcaagaactgggcaaatacgagcagtatatcggcggagggggttcaggtgggggtggctccgggggaggcggatcaatgagcgacaatggaccccagaaccaaagaaacgctccccgcatcacatttgggggaccctctgactcaaccggaagcaaccagaatggtgaacggtccggcgccaggtcaaagcagaggagaccccaggggcttccgaataatactgcctcctggttcactgccctcacccagcacggaaaggaggacctcaagtttcctagaggacagggagtgccaatcaacacaaattcaagcccagacgaccaaatcggctattatagacgcgccactcgacgaattcgcggaggagacggtaaaatgaaggatctttctccccgctggtatttttactatctcggaacaggaccagaggcaggactcccttatggagctaacaaggacggtatcatttgggtggccacagagggggcccttaacacacccaaggaccacattggtacaaggaatcccgctaacaacgcagcgattgttctccaactgcctcagggaaccaccctccccaagggtttctacgctgaggggagccgcgggggcagtcaggcgagctcacgctcatcttccagaagtcgaaatagctcccggaatagtacacctggttcaagtcgcggaacttcccctgcacggatggccggcaatggcggagacgctgcccttgcactgctgctgcttgacaggcttaaccagctggaatccaaaatgtccggtaagggtcagcaacagcagggacagaccgtgacgaaaaaaagtgcagccgaggccagtaaaaaaccaagacaaaagcggaccgcgacaaaggcctacaatgtgacccaggctttcggccggcggggcccagagcagacacaaggtaatttcggcgaccaggagctgatcagacaaggaactgactacaagcattggccgcagatcgcgcaattcgcaccttctgcatccgccttcttcggtatgtcacggattggaatggaagtgacccctagcggaacctggctgacgtacacaggagccataaaactggatgacaaggacccaaacttcaaagatcaggtaatcctcttgaataagcacatcgacgcgtacaagacctttcctccaaccgaacccaaaaaagacaaaaagaagaaagctgacgagacccaagcgctgccccagagacagaagaagcaacagaccgtgactctcttgcccgccgcagatctcgatgacttctccaaacagctgcaacagtcaatgtccagtgctgacagcacccaagcctaa

SEQ ID NO: 17 - protein, 1103 aa, artificial
  Fusion protein including S1-RBD, S2-HR2, M, and N
MFVFLVLLPLVSSQCFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFGGGGSGGGGSGGGGSYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIGGGGSGGGGSGGGGSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIAGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 18 - DNA, 3312 bp, artificial
  Nucleotide sequence encoding SEQ ID NO17
atgttcgtgttcctggtgctgctgccattggtgtcctcccagtgctttacggtagaaaagggaatttaccagacgtctaacttccgagtccagcccaccgagtctatcgtgcggttccccaacataaccaatctgtgcccattcggtgaggtgttcaatgccacacgcttcgctagttttactgtggaggtctatgcatggaatcgcaaacggattagtaactgcgtagctgattatagcgtgctctacaacagtgcatctttctctacgttcaaatgttatggcgtgtccccgaccaaactgaacgatctgtgcttcaccaatgtttacgccgactcattcgtcatcagaggggatgaagtgaggcaaatcgcccctggtcaaaccggtaagatcgctgactacaattataaacttcctgacgacttcaccggttgtgtgatagcatggaattccaacaatctggactccaaagtaggtggaaactataactatctctatcgactgtttagaaaaagtaatctgaaacccttcgagcgcgacatatccaccgagatttatcaggccggcagcactccctgcaacggcgtagagggatttaattgctatttcccattgcaaagctacgggttccagcccacaaatggcgtgggttaccaaccctacagggtcgtggtcctttcctttgaacttttgcacgcacccgctacagtttgtgggcccaaaaaaagcacaaatctcgtgaaaaataaatgcgtcaacttcaatttcgggggtggcggctccggcggcggtggctccgggggagggggatcttaccatttgatgagctttcctcagtctgcacctcatggggttgtctttttgcatgtgacctacgtccctgctcaggaaaagaattttaccaccgcccccgctatttgccacgatggtaaagcgcattttccgagggaaggggtgttcgtctccaacggaacccactggtttgtcactcagagaaatttttacgagccccagatcatcacaacagataacacctttgtaagcggtaactgtgacgtggtcataggtatagttaataacacggtctatgatcctctgcaacccgagctggacagcttcaaggaagaactcgataagtattttaaaaaccacacttcacctgatgtcgatcttggcgatatctccgggataaatgccagcgtagtcaacatccagaaagagatagataggctgaacgaggtcgctaaaaatctcaacgaatccctgatagatctgcaggagttgggtaagtatgagcagtatataggggggggcggcagtggaggcggagggtcaggaggcggcgggtctatggccgactccaacggaacaataaccgtcgaggagctgaaaaaactcctggaacagtggaatcttgtgattggatttctcttcctcacatggatttgcctgttgcagttcgcctatgccaataggaatagatttctttacatcatcaagctgatcttcctctggctgctttggcctgtgaccttggcatgttttgtgctggcggctgtgtacagaattaattggattaccggaggtatagccattgccatggcttgtctggtagggctgatgtggttgagctactttatagcctccttccgcctgtttgcgcgaacaagaagcatgtggtctttcaatcctgaaacgaacatcttgcttaatgtgcccctgcacggtacaatccttactagaccactgctggagtctgaactcgtgatcggagccgtgattcttcgcggacatctgagaatcgctggccaccacctgggccgctgcgacattaaagaccttcctaaagaaattaccgtcgccacatcccgcacactgtcatactacaaacttggcgccagccaacgcgtggctggagactccgggtttgccgcctatagccgatacaggatcggcaactataagctgaacactgaccattctagtagcagcgacaacattgctggagggggaggctccggtgggggtgggtctgggggagggggcagcatgtcagataacggccctcaaaatcagcggaacgccccgcgcataaccttcggaggcccaagtgactcaacaggaagtaaccagaacggagagaggagtggcgcgcgcagtaaacagagacgacctcagggcctgccaaacaacacagcttcttggttcaccgccctcactcagcacgggaaggaagatctcaagttcccaagagggcagggagttccaattaacaccaacagcagccccgatgatcaaatcggctactatcgaagggctacaagaagaattcggggcggtgatggaaagatgaaggatctgtccccaagatggtacttttactaccttggaacaggccccgaagcaggtctgccgtatggtgcaaacaaggatggaattatttgggtcgccacggaaggagccctgaatacacctaaggatcatatcggcacccggaaccctgctaacaatgcagcaattgtgctgcagctcccccagggcactacgctgccaaagggattctacgcagaaggcagtaggggtggatctcaagcttcctcaaggtcttctagccggtcaagaaactcaagtagaaacagtaccccaggatctagccgcggaacatccccagcacgcatggccggaaatgggggcgatgcggccctcgctcttcttcttcttgatcgcctcaaccagttggagtccaaaatgagcgggaagggccaacagcagcagggacagaccgtaaccaagaaaagcgcagccgaagcgtctaaaaaacccagacagaagcgaaccgcgacaaaagcatataacgttacgcaagccttcggaaggagaggccctgaacaaacacagggaaacttcggagatcaagaactgattagacaagggactgactacaaacattggccacagatcgcacagtttgccccctccgcgtctgccttttttgggatgtctcggataggcatggaagtcactccctccgggacctggttgacatataccggagcaataaaactggatgataaagaccctaattttaaggaccaagttatactgttgaacaagcacattgatgcttacaagacattcccccctacagaaccaaagaaagacaaaaagaagaaagccgatgaaacacaggctctcccccaacgacaaaaaaaacaacagacggtgactttgctccccgcggccgatcttgatgacttctccaaacagctgcaacagagcatgtctagcgctgatagtacccaggcttag

SEQ ID NO: 19 - protein, 2217 aa, artificial
  Fusion protein including S1-RBD and Nsp3
MFVFLVLLPLVSSQCFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFGGGGSGGGGSGGGGSAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGG

SEQ ID NO: 20 - DNA, 6654 bp, artificial
  Nucleotide sequence encoding SEQ ID NO19
atgtttgtgttcctggtcctgctccccttggtgtcttctcagtgctttacagtggagaaagggatttaccaaacctcaaactttcgggtgcagcccaccgaatcaattgtgcgctttccaaacattaccaatctgtgtccctttggcgaagtattcaacgccaccaggtttgcaagttttaccgtagaagtctatgcttggaatcggaaacgaatctctaattgtgtagccgactactctgtgctttacaatagtgcgtcattctctaccttcaagtgctacggagttagtcctacgaagcttaacgacctgtgcttcactaacgtatacgcggatagttttgtgattcgcggagatgaggttcggcagatcgctcctggacagacaggaaaaattgcggattacaattacaagctgcctgacgacttcacggggtgcgttatcgcatggaattccaacaaccttgacagcaaagtgggcgggaactacaactatctgtaccggctctttagaaagagtaacctgaagcccttcgagcgggacataagtaccgagatatatcaggccgggagcactccctgcaatggcgtagaaggattcaattgctattttcccctgcagtcctatggatttcagccaactaatggagtgggttatcagccttatcgggtcgtggtccttagttttgagctcttgcatgcgcctgccactgtgtgcgggccaaaaaagtctacaaaccttgttaagaacaaatgcgtgaattttaatttcggcggaggcgggagtggcggtgggggcagtggtgggggcggctcagcccctactaaggtcaccttcggggacgacaccgtaattgaagtacaaggatacaaaagtgtgaacattaccttcgagctggacgagcggattgacaaagtgctgaatgagaaatgtagtgcttacacagtagagcttggcactgaagtgaatgaattcgcatgcgttgtggctgatgccgtgataaagaccctgcagcccgtgagcgagctcctcacccctctgggcatcgacctggacgagtggagcatggcgacctactacctgttcgacgaatctggcgaatttaagctggcttctcatatgtactgtagcttctatccccccgatgaggacgaggaagagggagattgcgaagaggaagaatttgaacccagcactcagtatgaatatggaactgaagatgattaccaaggtaaacctctggaatttggagcaacgagtgcagctctgcaacccgaggaggagcaggaggaagactggctcgacgatgattcccaacagacggtcggccaacaggatgggtccgaggataatcagacgaccactatccagactatagtcgaggttcagccacaactggaaatggagcttactccagtagtgcagaccatagaggtcaatagctttagcggatacctgaaactgactgataatgtctacattaagaatgcagatatagtcgaagaagccaaaaaggtgaaacctaccgtggttgtcaatgccgccaacgtctacctgaaacacgggggaggcgtagccggcgccctgaataaagcaacaaacaacgccatgcaggtagagtcagatgactacatcgcaaccaatgggcctttgaaggtgggaggcagctgtgtcctgtctggccacaatctggccaaacattgtctccacgtggttggaccgaatgtgaacaagggcgaggatattcagttgctcaagagcgcatatgagaattttaaccagcacgaggtactgttggccccactgcttagcgcagggattttcggcgctgaccctattcatagtcttcgagtgtgtgtggatactgttagaacaaatgtctacctggcagtcttcgacaaaaatctgtatgacaaacttgtctcatcattccttgaaatgaagtcagaaaagcaagtcgagcagaagatcgcagagatcccaaaagaggaagtgaagccatttatcaccgagagcaaacccagtgtggagcaaaggaaacaggatgacaagaaaattaaggcatgtgtggaggaagtcaccacaactctggaggaaacgaaattcctgacagagaatctcctcttgtatattgacattaatggaaaccttcaccccgacagcgcaacgctggtctctgacatcgatattacgtttcttaagaaagatgctccttatatcgtgggcgacgtcgtgcaagaaggagtgctgaccgccgttgtcatcccgacaaagaaggccggcgggactacagaaatgctggcaaaggccctgcggaaagtgccaacagataattacatcacgacctatcctgggcaaggcctcaacggctacaccgtggaagaggccaagacagtgctgaaaaagtgcaaaagcgccttttatatcttgccctctattatcagtaatgagaaacaggaaattctgggaactgtgagctggaacctgagagagatgctggcccacgcggaggaaacgagaaaattgatgcccgtgtgtgtggaaactaaggctatcgtgagcactattcagcggaagtacaaaggcataaaaatccaggaaggcgtggtggattatggcgctagattctatttttatacatccaagacgactgttgcatctctgatcaacacactcaacgatctgaatgagaccctggttactatgcctcttgggtacgtgacacatggtctgaacctggaagaagccgctcgatatatgaggagcctgaaggtccccgccaccgttagcgtctcctcccctgacgccgtgacagcctacaacggatacctgacctcctcctctaagaccccagaggagcacttcatcgaaactatctccttggccggaagctataaggattggagttattctggacagagcacacaattgggcattgagttcctgaagagaggcgacaagagcgtgtactacacctccaatcctaccaccttccatctggacggagaagtaattacatttgacaatctgaagacacttctcagccttagggaggtgcgcaccattaaggtattcaccaccgttgataacattaacctccatacccaggtggtggacatgagtatgacgtacggtcaacagttcgggccaacatatttggatggggcagacgtgaccaagattaagccccataattcccatgagggaaagacattctacgtcctgcccaacgatgacacccttcgagtagaggcatttgaatactaccacacaacagacccgtctttcttgggacgctacatgagcgcactcaatcacactaagaaatggaaatacccgcaggttaacggacttacctccattaagtgggcagataataactgttacctcgctacagccctgctgacattgcaacagatcgagctgaaattcaacccccccgcactccaagatgcctactaccgagcacgagccggcgaggctgccaacttttgcgccctgatcttggcttactgcaacaaaactgtaggagaactgggtgacgtgcgagaaacgatgagctatctgttccagcacgcaaatctggactcatgcaaacgagtactcaacgtggtctgcaagacatgcggtcagcagcaaactacactcaagggagtagaggctgtgatgtatatgggcacactgtcctacgagcagttcaagaagggcgtgcagattccttgtacttgcggtaagcaggccaccaaatatttggtacagcaggaaagcccattcgtgatgatgtccgcaccccccgctcagtatgaactcaaacatggtacattcacctgcgcctcagagtacacaggaaattaccagtgcggccactataagcatatcacctcaaaggagaccctgtattgtattgacggcgcccttctgaccaagagctctgagtacaagggcccaatcacagacgttttttataaggagaactcttacaccacaaccatcaagcccgtgacctacaagctggatggggtggtgtgcacagaaatagatccgaagctggataactattataaaaaagacaacagctacttcaccgagcaacctatcgacttggtcccaaaccagccatacccgaacgccagttttgacaattttaagtttgtctgcgacaacattaagtttgccgacgacctgaatcagctcactggatacaagaagccagcaagcagggagctgaaggtgacctttttccccgacctcaacggcgacgtggtggccattgactataaacactataccccaagtttcaagaagggcgccaaactccttcataagcctatcgtgtggcatgttaataatgcaactaataaagctacatacaaacctaacacatggtgtatacgctgcctttggtctactaaaccagtcgagactagtaacagctttgatgtgcttaaatccgaggacgcccaaggaatggacaaccttgcctgtgaagacttgaagccggtcagcgaggaggtggtcgagaacccgacaatacaaaaggacgtgcttgagtgtaacgttaagaccacggaggtagttggggacatcattctcaagccagccaataactcacttaaaatcaccgaggaggtgggtcacactgatctgatggctgcctacgtggacaactcctcacttactataaaaaaacccaatgagcttagcagggtgcttggtctgaagacacttgcaacccacgggcttgcggcggtcaattcagtgccctgggataccattgccaattacgcaaagccctttcttaataaagtcgtttctaccacaactaacatcgttaccagatgcctgaacagagtttgtacaaactacatgccttacttctttacactcctgctgcagctctgtacctttactcggagtacaaattcacgcataaaggcctccatgccaacaacgatcgcgaagaacaccgtgaagtcagtaggcaagttctgtctggaagcatccttcaactatctcaagtctcccaacttttctaagctgatcaacataatcatttggttcctcttgctgtcagtatgcctggggtcactcatctacagcaccgctgcactgggcgtattgatgtcaaatctgggaatgccatcttattgtactggatacagagagggatatctgaacagtactaatgtgacaattgccacctattgcactggtagcatcccttgctctgtatgcctttcaggcctggattcccttgatacatatcccagcctcgagaccattcagattacaatatcctcattcaagtgggatctgaccgcttttggactggttgctgaatggttcctggcttacatcctcttcacacgcttcttttatgtgctgggcctggccgctatcatgcaactgttcttcagttattttgctgtccacttcatctctaacagttggctgatgtggctcatcatcaacctggtgcaaatggcccctatttccgccatggtgcgcatgtatatatttttcgcatccttctactatgtttggaaatcttacgtgcatgtagtggacggttgcaacagttcaacttgtatgatgtgctacaaaagaaatcgagccacacgcgtcgagtgcacaacgattgtcaacggtgtccgaaggtccttctacgtgtacgccaatggaggtaaagggttctgcaagcttcataattggaattgcgtgaattgcgacacattctgcgccggtagtaccttcatctcagacgaagtggcccgcgatctgtccctccaatttaagcgacctataaatccaaccgatcagagtagctacattgtggactctgtgaccgttaagaacggcagcattcatctgtactttgataaagcaggacaaaagacgtacgagcggcactctctgtctcatttcgttaacctcgacaatctccgcgccaataacaccaagggaagtttgccaatcaacgtaattgttttcgatggtaagagtaagtgcgaagagtcttccgctaaaagtgcttctgtgtactactcacagttgatgtgtcagcctatccttctgctggaccaggccctggtgtccgatgtcggagattcagccgaggttgctgtcaagatgttcgacgcttacgtaaacaccttcagctctacgtttaatgtgcctatggagaaacttaagacacttgtggcaacagccgaggcagagctggcgaaaaacgtgtcactggacaacgtcctgtccacttttatatccgccgctcggcaggggttcgtggattccgacgtggagacaaaagatgtggtcgagtgcttgaaactgtctcaccaatccgatatagaagtgaccggcgacagctgcaacaactacatgctcacatacaataaggtggagaacatgactccgagggatttgggagcatgcattgactgttcagctagacatattaacgctcaagtggccaaaagccataacattgcactgatctggaacgttaaagacttcatgtccctgtccgagcagctccgcaaacaaatcaggtccgccgccaagaagaataacttgccctttaaactcacttgcgccacaacaagacaggtggttaatgtcgttactactaagattgcactgaagggaggctga

SEQ ID NO: 21 - protein, 872 aa, artificial
  Fusion protein including S1-RBD, S2-HR2, Ubl1- nsp3, 3Ecto-Nsp3 and Nsp8
MFVFLVLLPLVSSQCFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFGGGGSGGGGSGGGGSYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIGGGGSGGGGSGGGGSAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEGGGGSGGGGSGGGGSSNLGMPSYCTGYREGYLNSTNVTIATYCTGSIPCSVCLSGLDSLDTYPSLETIQITISSFKWDLTAFGLVAEWFLAYILFTRGGGGSGGGGSGGGGSAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 22 - DNA, 2619 bp, artificial
  Nucleotide sequence encoding SEQ ID NO21
atgtttgtgtttctggtgctgcttcccctggtatcatctcagtgcttcacagtggagaaagggatttaccagacgagcaactttcgggtccagccaaccgaaagtatagtgaggttccccaatattactaacctttgtcccttcggtgaagtgttcaatgcaacccgatttgcttctttcacggtcgaggtctacgcttggaataggaaaagaatctccaattgtgtggccgattactccgttctgtataatagtgcgtcattttccaccttcaagtgctatggtgtgtccccaacaaaattgaatgatctttgttttaccaacgtatacgcagacagcttcgtgataaggggggacgaggtgcggcagatagccccaggtcagaccggaaaaatagcagattacaattataagctcccagatgattttacaggctgcgtgatcgcctggaactctaataacctcgattcaaaggttggcggaaattacaactacttgtataggcttttcaggaagtctaacttgaagcccttcgaacgcgacattagcacagagatttaccaggccggttccactccttgtaatggtgtagaaggcttcaattgctacttccctctgcaatcttatggttttcagccaaccaatggggtgggctatcagccttatcgcgtggtggtgctgtcattcgagcttcttcatgctccagcaacagtgtgcggaccaaaaaaatctacaaacttggtgaagaataagtgcgttaatttcaattttggcggaggaggatcaggaggcggcggcagcggcggcgggggttcttaccacctcatgtctttcccgcagtctgctccccacggcgttgtatttctgcatgtcacttatgtccctgctcaggagaagaacttcacaacagcgcctgcaatttgccacgatggaaaagcacacttcccccgagagggcgtcttcgtcagcaacgggacccactggttcgtgactcagaggaatttctacgaaccgcagatcataacaaccgacaacacgtttgtgtcagggaactgcgacgtcgtgatcgggatagtcaacaacaccgtatacgatcctctccagcctgagctggacagctttaaggaggagctcgataaatattttaagaatcacacttctccggacgtggacctcggcgatatttctggcattaatgcttcagtggtgaacattcaaaaggagattgacaggctgaacgaggttgccaagaatctgaatgaatctctcattgaccttcaggaactggggaaatatgaacaatatattggtggcggaggatccggaggcggaggatcaggcggcggaggaagcgcccctacaaaggtcactttcggagatgacaccgttattgaggtgcagggctacaaatctgtgaatattacctttgagctggatgaaagaatcgacaaagtgttgaatgagaaatgctccgcttatactgtcgaactggggactgaagttaatgaattcgcgtgtgtggtggcagacgccgttataaaaactttgcagcctgtatccgagttgctgacccctttggggatcgatcttgatgaatggtccatggcaacatactacctctttgacgagtcaggagaattcaagctggcctctcacatgtactgttcattttatccacccgacgaaggaggcggagggagcggtggcggtggttctggcggaggcggttcatcaaatctcggaatgccaagctactgcactgggtatagggagggttaccttaattctacaaacgtgaccatagccacttactgcactggatcaataccttgcagcgtgtgtctcagcgggcttgactccctcgatacatacccctcactggaaactattcagatcactatatcttccttcaaatgggacctgactgccttcgggctggtcgccgaatggtttctggcctacattctttttactaggggaggcgggggctccggtggaggaggctccgggggcggcggcagcgcaattgcctcagaattctcctctctgccatcatacgctgctttcgcaactgcccaggaagcctacgaacaagctgttgcgaatggggactccgaagtggtgcttaagaaactgaaaaaatctctcaacgtcgcgaagagtgaattcgatcgagacgcagcaatgcagaggaagctggagaagatggccgatcaagcaatgacacaaatgtataaacaggcccgaagtgaggataagcgggcgaaggtcacatccgccatgcagactatgctctttactatgctgagaaagctcgacaatgacgccctgaacaacattattaataatgcaagggatggttgtgtgcccctcaacattatacctctgactacagccgctaaacttatggtggtgattcctgattacaatacttataaaaatacatgtgacggcacaactttcacatatgccagtgcactgtgggagattcagcaggtggttgacgcagacagtaagattgtgcaactgtcagaaattagtatggataatagcccgaacctcgcgtggccactgattgttaccgccctgagggctaattcagctgtcaagctgcagtag

SEQ ID NO: 23 - protein, 902 aa, artificial
  Fusion protein including S1-Spike protein and Nsp8
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARGGGGSGGGGSGGGGSAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 24 - DNA, 2709 bp, artificial
  Nucleotide sequence encoding SEQ ID NO23
atgttcgtcttccttgtgctcctgcctctggtgtcatcccagtgcgtaaacctgacaacaagaacccagcttcctccagcctacactaattccttcactagaggggtgtactaccccgataaagtatttaggtcctccgtgctccactccacacaagatcttttcctcccgttcttctccaatgtcacatggtttcatgcgatccatgttagtgggacaaatggcacaaaacgctttgataacccagtcctcccttttaatgacggggtgtatttcgcatctactgagaaaagcaacatcatcagaggatggattttcgggaccacactggattctaaaacacagagcctgctgatagtaaacaatgcaactaatgtggtgatcaaagtgtgcgaatttcagttctgcaacgacccattccttggcgtttactatcacaagaacaataaaagttggatggagtccgaatttagagtgtattcaagcgctaacaactgtactttcgagtacgtgtcccagccatttctgatggatctcgaaggcaaacagggcaactttaaaaatctgcgggaattcgtcttcaagaatatcgacgggtacttcaaaatctattccaaacatacccccataaaccttgtgagggacctgccccaaggatttagcgcattggaacccttggtggacctgcctattggaatcaatatcactaggtttcagacactgctggccctgcaccgctcctaccttacgcctggggactcctcatccggttggaccgctggcgcagctgcttattatgtgggttacctgcaaccccggacattcctccttaaatacaatgaaaacgggaccataacggacgccgtcgattgtgccctcgatccactcagcgagacgaagtgcacactgaagtccttcacagtggagaaggggatatatcagaccagtaattttcgggtccagcctacagagtcaattgtgcgctttccaaatatcaccaacctttgtccattcggcgaggtttttaatgcaaccagattcgcatcctttaccgtagaagtttatgcttggaatcgaaagaggatttcaaattgcgtcgctgactatagcgtgctgtataatagcgcttcattttcaaccttcaaatgttatggagttagcccaaccaagttgaatgatctttgcttcacaaatgtgtatgccgactcatttgttatccgcggagacgaggtcagacaaatcgcccccggacagacgggcaagatcgcagactataactacaaactccccgacgattttaccgggtgcgtgattgcctggaactctaataaccttgatagtaaagttgggggaaattataattatctgtacaggctctttcggaaatcaaacctgaaaccattcgagcgggatatttccacagagatctatcaggctggctcaacgccttgcaacggggtagagggatttaactgttattttccgttgcaatcctatggtttccaacctaccaacggtgtgggatatcagccgtatagggtggtcgtgcttagcttcgagctgctgcacgctccagccacagtctgtggcccgaaaaagtcaactaatcttgtgaaaaataaatgcgtgaattttaatttcaatgggttgaccggaacaggagttctgaccgagagcaataaaaagtttttgccctttcagcaatttggccgggatatagccgacacaaccgatgcggtccgcgatcctcaaacactggaaattttggatatcaccccttgtagctttggtggcgtctctgtcatcacccccggaaccaatacttctaaccaggtggccgtcttgtaccaggatgttaactgtaccgaggtgcctgtggcgattcacgcagaccagcttacccccacatggagagtgtattctacaggatctaacgtcttccagacacgagcgggctgcttgattggcgctgaacacgtgaataattcctacgagtgtgacattccgataggcgcgggaatctgcgcatcatatcagacacagactaatagtccgaggagagctagagggggcggcgggtcaggggggggaggcagtgggggagggggctcagcgatcgcgtccgagttttctagtctgccgagctatgctgcattcgccacagcccaagaagcatatgaacaagccgtcgctaacggtgactctgaggtggtgctgaagaagctgaagaagagccttaatgtggcaaagagcgagttcgacagggacgccgcaatgcagcgcaagctggaaaaaatggctgaccaggccatgacccagatgtataagcaggctagatcagaagataagagagccaaagtgacttccgcgatgcaaaccatgcttttcacaatgctgcggaaactggataacgatgctctgaataacataattaacaatgcccgagacggctgtgtccctctcaatattatcccccttaccaccgcggcaaaacttatggtggtgatacccgactacaacacttacaagaacacatgcgacgggacgacattcacgtacgcgtccgctctctgggaaattcaacaagtggttgacgctgattccaagattgtgcagctgtcagaaatctcaatggataattcacctaatttggcctggcctctgatcgtgactgcattgagggcaaattccgccgtcaagttgcagtga

SEQ ID NO: 25 - protein, 1277 aa, artificial
  Protein including full spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - DNA, 3834 bp, artificial
  Nucleotide sequence encoding SEQ ID NO25
atgttcgtcttcctggtcctcctccctcttgtctcttctcagtgtgtgaacctcactacaaggacccaactccccccagcttatacaaactccttcacgcgaggagtgtactaccccgataaagtcttcagaagcagcgttctccatagcacgcaggatctgttcctgccgttcttttctaacgtaacttggtttcacgccattcatgtatcaggcactaatggaaccaaacggtttgacaatcctgtgttgccctttaacgacggggtttacttcgcctctaccgagaaatcaaacatcatcaggggatggattttcggcactacccttgactctaagacacagagcctcctcatcgtgaataacgccaccaacgtagttatcaaggtgtgtgaatttcagttctgcaacgacccgttcttgggagtatattaccacaaaaacaacaaatcatggatggagtccgagtttcgggtgtactccagcgccaataactgcacattcgagtacgtttcccagcccttccttatggaccttgaagggaagcagggtaacttcaaaaaccttcgggaattcgtatttaaaaacatcgatggctattttaagatatactctaaacacacacctatcaacctcgtgagagaccttcctcagggattctctgctcttgagcctctcgttgatttgcctataggcataaatattacccgattccagaccctgcttgctctgcacagatcctatttgacccccggcgatagctcctccgggtggaccgccggcgcagccgcctattatgtggggtatctgcagcccagaaccttcttgctcaagtacaacgagaatggaacaataaccgatgcagtggactgtgccctcgatccccttagcgagactaaatgcacgctgaagagctttaccgtcgaaaagggaatctaccagacaagcaattttcgggtccagccaaccgagagtatcgtgagatttccgaacattaccaacctgtgccctttcggggaggttttcaacgctacccgctttgcttccttcacagtcgaagtgtacgcctggaaccgaaagcggatctcaaattgtgtagccgactatagtgtcttgtataatagcgcctctttttcaacatttaaatgctatggagtgagtccaacgaaacttaatgacctctgtttcaccaacgtgtacgcggatagcttcgtgatcagaggagacgaggtgagacaaatcgcacctggccagacaggaaagatagctgactacaactataagctgcccgatgattttacgggatgtgtgatagcctggaactcaaacaacctggactccaaggtcggaggtaattataattatctgtaccggctcttcaggaaaagtaacctcaaacctttcgagcgggacatttctacggagatctaccaggctggcagcaccccctgcaatggagtagagggtttcaactgttattttcctttgcagagctatggctttcagcccaccaacggagtgggttaccaaccctacagagtggttgtactgtccttcgagcttctccacgcgcccgcaactgtgtgcggtccaaagaaatctactaaccttgtgaagaacaagtgcgtcaatttcaatttcaacgggcttaccggaactggtgtcctgacagagtctaataagaaattcctgcccttccagcaattcggacgcgatatcgctgacactactgatgcagtgagggatccccagacactcgagattctggatatcaccccatgctccttcggaggggtatcagtaatcactccaggcacaaacacaagtaatcaggtggccgtgctttatcaggatgtaaattgcactgaagtgcctgtggccattcacgccgaccaactcacccccacatggcgagtgtacagcaccggcagtaacgtattccaaactcgcgcaggctgtctgattggcgcagagcacgtgaacaacagttatgagtgtgatattcctattggggccggcatatgcgcttcttaccagacccagacaaattctcctcggcgcgccagatcagtagccagtcaatctataatcgcgtataccatgtctttgggcgccgagaactccgtcgcttactccaacaacagtattgccattccgaccaatttcaccatttcagtcaccacagaaattcttcccgtgtcaatgaccaagactagcgtagattgcacgatgtacatttgtggcgacagtacagaatgtagtaacctcctgttgcaatatggaagcttctgtacgcaattgaacagagccttgactggtattgctgttgagcaagacaaaaatacacaggaggtgttcgcccaggttaagcagatttacaagactccccccatcaaggacttcggtggtttcaacttctctcaaattctgccagatcccagtaagcctagcaagcggtccttcattgaagacctgctgttcaataaggtgacactggccgatgcgggctttataaagcagtacggcgattgcctgggagatatcgccgcaagagatctgatatgcgctcaaaaatttaatgggttgactgtcctgccccctctgctcacggacgagatgatcgcacaatacactagcgccctcctggccggtacgataacatctggctggacattcggcgcaggggccgccctgcagatacccttcgctatgcagatggcatataggttcaatggcattggggtaactcagaacgtactgtatgagaatcagaagctgattgcgaaccaattcaattctgctatcggtaagattcaggactcactgagctccaccgcatccgcactcggcaagcttcaggatgttgtaaatcagaacgctcaggctttgaataccctggtaaaacaactctcatcaaacttcggcgccatctccagcgtactcaatgacattctcagccggttggacaaggtggaggcggaagttcagatcgacagactgatcaccggccgactccagtcactgcagacgtacgttacacaacaactcattcgggccgctgagattcgggccagtgccaacctcgccgctacgaagatgagcgaatgcgtgctcgggcagtctaagagggttgacttttgcggcaagggctatcatctgatgagttttccgcaatccgctccccatggagttgtattcctccatgtgacatatgttcccgctcaggaaaagaactttaccacagctcctgccatctgtcatgacggcaaggcccattttcctcgagaaggggttttcgtttctaacggaactcactggttcgtgacccagcgcaatttctacgaaccgcagattatcacaacagacaatacttttgtgtcaggaaactgtgatgtggtaataggcatagtcaacaatacagtttacgacccccttcagcctgagctggatagctttaaggaagaactggataagtacttcaaaaaccatacctctcccgacgtagatcttggggatatctctggcattaatgcttccgttgtgaatatccagaaagaaatagaccggcttaacgaagttgccaaaaatttgaacgaaagcctgatcgatcttcaagaattgggtaaatacgaacagtacataaaatggccctggtatatctggctgggcttcatcgctggcttgatagctattgtcatggttacgattatgctctgctgtatgacaagctgctgcagctgtctcaaaggatgttgttcatgcgggtcatgttgcaaatttgatgaagacgattcagagccagtattgaaaggcgtgaaactgcactatacctag

SEQ ID NO: 27 - protein, 1713 aa, artificial
  Fusion protein including full spike protein, UBl1-Nsp3, 3Ecto-Nsp3,and Nsp8
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGGGGSGGGGSGGGGSAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEGGGGSGGGGSGGGGSSNLGMPSYCTGYREGYLNSTNVTIATYCTGSIPCSVCLSGLDSLDTYPSLETIQITISSFKWDLTAFGLVAEWFLAYILFTRGGGGSGGGGSGGGGSAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 28 - DNA, 5142 bp, artificial
  Nucleotide sequence encoding SEQ ID NO27
atgtttgttttcctcgtgctcttgcctcttgtgagctctcagtgcgtgaatttgaccacaagaacacagcttcctcccgcatacaccaatagtttcaccaggggcgtgtactatcctgataaggtcttcaggagctcagtgcttcatagcacccaagatcttttcctcccattttttagtaatgtcacttggtttcatgcaattcatgtatccggaacgaacgggacgaagcgcttcgataatcctgtgctcccttttaacgatggggtttattttgcctctacagagaaatccaatattatcagaggatggattttcggcactactcttgactctaagacacagtccctgttgatcgtgaacaacgccactaatgtggtgattaaagtgtgtgaatttcagttttgtaacgaccctttcctgggggtctattatcataagaataataaaagctggatggaatccgaattccgggtgtatagctctgccaacaactgcacctttgaatatgtaagccagcccttcctcatggaccttgagggcaagcagggaaactttaagaatctgcgcgaatttgtgtttaagaacatcgacggctacttcaaaatctattctaaacatacaccgatcaatttggtgagagaccttccacaaggattctccgccctggagcccctggtcgatctgcccatcggaatcaacatcactaggttccagactttgctggccctgcataggtcatatctgacccccggggattcatcttctggatggaccgcaggtgcagctgcctattatgtgggctatttgcaacccaggaccttcctgctgaaatataatgaaaatggtacaatcactgacgctgtagactgcgcacttgatcctctgtccgagacaaaatgtacactgaagtcattcacggtggaaaaaggcatttatcagacatctaactttcgggtacagcctacagaaagtatcgtgagattccctaacattaccaatctctgtccttttggagaggtgtttaatgccacacgatttgcatccttcacagtggaggtatacgcctggaaccgaaagcggatctccaactgcgtcgccgactattctgtcctgtataattcagcctcattcagtaccttcaagtgttatggcgtgtccccaaccaaattgaatgacctgtgtttcaccaatgtgtacgcggattctttcgtgattcggggtgacgaagtgagacagatcgcgccaggacaaacagggaaaatcgcggattataattacaagctgcccgatgacttcactggatgtgtgattgcttggaactcaaacaatttggacagtaaagttggtggcaattataactacctgtatagactctttaggaagtcaaacctgaagccttttgaacgcgatataagcactgagatctatcaggcagggtctactccgtgcaatggggtagaggggttcaattgctattttcctctccaatcatacggctttcagccgactaatggcgtgggctatcagccttacagggtagtcgtcctgagctttgaactgctccatgcaccagccacagtatgtgggccgaagaaatccacgaacctcgtgaagaataaatgcgttaacttcaacttcaatggcctcacaggcacaggagttctgactgagtctaacaagaagttcctcccattccaacagttcggccgcgacattgctgatacaactgacgctgtcagagatcctcagacccttgagatactggatataacaccatgctcattcgggggcgtcagcgtgatcacacccggcaccaatacaagtaaccaggtcgcggtgctgtatcaggatgtgaactgtactgaggtccctgtcgccattcatgccgatcagctgactcctacatggagggtgtactctacgggatctaatgtctttcaaacacgcgctggctgtcttataggggccgaacatgttaataactcatacgaatgcgacatacctatcggcgccggaatttgcgcctcatatcaaacacaaaccaatagcccccgccgcgcgaggagcgttgctagtcaaagcatcattgcctacactatgtcactcggggcagagaattccgttgcctacagcaacaatagtattgcaattccaactaatttcaccattagcgtgactaccgaaatcttgcctgttagcatgaccaagacctctgtggattgtacaatgtatatatgcggggacagcactgaatgttcaaatttgctgttgcaatatgggtcattctgcactcaactcaacagggcactgaccgggattgctgtggagcaggacaagaacacccaagaggtattcgctcaggtaaaacaaatttacaaaaccccacctattaaggatttcggggggttcaatttttctcagatcctccccgaccccagtaaaccctcaaagcggagcttcattgaagacctgctctttaacaaggtaactctggcggatgccggctttattaagcagtacggagattgtctgggtgatatcgccgctcgagacctcatttgcgctcaaaaatttaatggtcttacagtactgccaccactgctcactgacgagatgatagcccagtacacatctgccctccttgcgggcactatcacaagcggctggacgttcggcgcaggagcggcgctgcaaattccattcgcaatgcagatggcctatagatttaatggcattggcgttacacaaaatgttttgtatgagaaccagaagctgattgccaaccaattcaatagcgctatcggtaaaatccaggacagcctgtcctcaacagcatcagccctggggaagttgcaggacgtggtaaatcagaacgcacaagccctcaatacccttgtcaagcagctgagtagtaactttggggcaatttcctccgtgctgaacgacatcttgtcacgactggacaaggttgaggccgaagttcagattgaccggctcattaccgggagactccagtccttgcaaacgtacgtcacccagcagttgatcagggctgcggagatcagagccagtgctaacctggcggcaaccaagatgagcgagtgtgttctcggccaatccaagcgggtcgatttctgcggcaaaggataccacctgatgtctttcccgcaaagcgcgccccatggggtggtctttcttcatgtgacgtatgttccggctcaagagaagaattttacaaccgcccccgcgatttgtcacgacggtaaagcccacttcccaagagagggagttttcgtgtctaatggaacccattggtttgtgactcagcgcaatttctacgaaccccagatcataacgacagataacacgttcgtgtctggtaattgcgatgtagtgattggcatagtcaacaataccgtatatgaccctcttcagcccgagctcgatagctttaaagaagaactggataaatatttcaagaatcacactagccctgacgtcgacctcggtgacatctccggaattaacgcttccgtggtaaatattcagaaggaaattgaccggctcaacgaggtggctaaaaacctgaacgagagtctgattgatctccaagaactggggaaatatgagcaatacatcaagtggccctggtatatttggctgggcttcattgcaggcctgatcgcaatagtgatggtgaccattatgctgtgctgcatgacgagctgttgttcatgtcttaagggttgttgctcttgtggatcttgctgtaagtttgacgaggacgattccgagccagttctgaagggagtgaaactccactataccggaggcggtggcagcggaggaggaggctcaggcggtggcgggtcagctcctacaaaggttaccttcggtgatgacacggtcatcgaggtccaggggtataagtccgtcaacattacgtttgaactcgacgagcggatcgataaagtgctgaacgaaaaatgcagcgcttacactgtcgaactggggacagaggtgaatgaatttgcttgtgtggttgctgatgccgtcatcaagaccctgcaacctgtgtccgagcttttgactcctttgggaatcgatcttgatgagtggagcatggcaacgtactatctctttgacgagagcggggagttcaaactggccagtcacatgtactgctccttttaccctccggacgaaggtggcggaggctcagggggaggtggtagcggtgggggtgggagctccaatctgggaatgccgagttactgcactgggtatagggaaggttaccttaacagcactaatgttaccatcgctacgtattgcacaggcagtatcccttgttcagtgtgtcttagcggtctggattccctcgatacatacccctctctcgaaacaatacaaatcacgatctcatcttttaagtgggacctcaccgcgtttggcctggttgctgagtggttcctggcgtatatcctgttcactagaggcggaggaggatccggaggtggaggttctggaggcgggggatctgccatagcttccgaattctcatccttgcccagctatgccgcatttgccactgctcaggaggcgtacgagcaggccgtggccaacggtgattccgaggtggttcttaagaagctgaagaagagtcttaatgttgctaagtccgagtttgatcgcgatgccgcaatgcagaggaaactggaaaagatggcagaccaggctatgactcagatgtataaacaggcccgcagtgaggacaaacgggctaaagtaaccagcgcaatgcagacaatgttgtttacgatgctgaggaaattggacaatgacgctctcaataacattattaataacgctagggacggatgtgtgcccctgaacatcatcccccttactacggcggccaaattgatggtggtgattcctgattataacacatacaagaacacttgcgacggtacaacttttacttatgctagtgcactgtgggagatccaacaagtggtggatgctgattccaaaatagtacaattgtctgagatcagtatggataattccccaaaccttgcctggcctttgatagtgactgccctgcgagccaacagtgccgttaagctgcaatga

SEQ ID NO: 29 - protein, 1569 aa, artificial
  Fusion protein including S1, M, N, and Nsp8
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASFTVEVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARGGGGSGGGGSGGGGSMADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIAGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGSGGGGSGGGGSAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 30 - DNA, 4710 bp, artificial
  Nucleotide sequence (SEQ ID NO30) encoding SEQ ID NO29
atgttcgtgtttcttgtgctgctgccactggtctcttcccagtgcgttaacctcactacgagaacacaactccctcctgcttatacgaatagtttcacgcgcggtgtttattaccccgacaaggtgttccggtcatccgtgcttcactcaacgcaagaccttttcttgccctttttctccaacgtgacttggttccacgccatccatgttagcggcacaaatgggaccaagcgattcgacaatcccgtactgccctttaacgatggcgtgtactttgcttccactgagaagtccaacatcattcgcggatggatattcggcaccaccctcgacagcaaaacacagtctctgctgatcgttaacaatgcaaccaatgtggtcatcaaggtctgcgagtttcagttctgcaacgaccctttcctcggcgtctattatcataaaaataacaaaagttggatggaatcagagttcagagtgtactcctcagctaataactgcacgtttgagtacgtctcacaaccttttctcatggacctcgagggtaaacaggggaatttcaagaatctgcgagagttcgtcttcaaaaatatcgatggatacttcaaaatctatagtaaacacacaccaatcaacctggtcagggacttgccccagggtttttccgcactggagcctctcgtggatcttcccatcgggatcaatatcacccggttccagactctcctggctcttcatcgcagctatctgactcctggggatagcagtagcggctggaccgctggcgccgctgcctactacgtcgggtatctccagcctcgaacctttttgctgaaatacaatgagaatgggactatcactgatgcagttgactgcgccctggaccccctgtctgagactaagtgtaccctgaaaagcttcactgttgaaaaggggatatatcaaacatccaacttccgggtacagccaactgaaagcatcgttaggtttcccaatattacaaacctgtgcccttttggggaagttttcaacgctactagattcgctagttttaccgtggaagtttatgcttggaaccggaaaaggatttcaaattgtgttgcagattatagcgtcctgtataatagtgccagcttctctacgtttaagtgttacggcgtgagccccaccaagctgaatgacctgtgtttcactaatgtgtatgcagacagctttgtcattagaggggatgaagtaagacagatcgcccccggccagaccgggaaaattgccgactataattacaagctgcccgacgactttaccggctgtgttatcgcttggaactcaaataacctcgattccaaggtaggagggaactataactatttgtacaggctcttcagaaaaagcaacttgaagcccttcgagagggatatcagcaccgagatttatcaagctggatcaacaccttgtaatggcgtggagggattcaactgctacttcccactgcaaagctacggctttcagccaacaaatggggtcgggtatcagccttacagagtagtggtcctcagttttgaactgctccacgctcctgcgacagtatgtggccctaagaagtctactaacctcgtgaaaaacaaatgtgttaatttcaatttcaatggactgacaggcacaggcgtgctcacagaaagtaataaaaagttcctgccctttcagcagtttggccgagacatcgctgataccaccgacgctgtgagagatccccagacgctcgagattctggatatcaccccttgcagctttggcggggtgtcagtcatcacccccggcactaatacatccaatcaggtggccgtactctatcaggacgtcaactgcacggaggtcccggttgccatccacgccgaccaattgactcctacgtggcgagtgtattccactggaagcaacgtatttcaaacacgagctggttgtctgatcggagccgaacacgtgaataactcttacgaatgcgacatcccaataggcgctggcatctgcgcatcttatcagactcagacaaacagtcccagaagagcaagggggggtggtggttctggcggagggggttcaggggggggaggctccatggccgattccaatggtactattaccgtagaggaactcaagaaactgctcgaacagtggaatctcgtgatcggatttctctttctcacctggatatgcttgcttcaattcgcctacgccaatcgcaaccgatttctctacatcatcaagttgatctttctttggcttctgtggcccgtgacactggcatgctttgtgctggccgctgtttatcggatcaattggataacggggggaatcgccattgcaatggcctgtctcgtgggactcatgtggttgtcctacttcatcgcaagttttaggctgtttgctaggacccgcagcatgtggagttttaaccccgaaaccaacattctgctcaacgtgccccttcacggtacaatcttgacaaggcctctgctcgaatcagaattggtgatcggcgccgtgatcctgcgcggacacctcagaatcgcaggacatcatttgggcaggtgtgacatcaaggatctccccaaagaaataactgtggccacttctcgcacactgagttactacaagctgggtgccagtcagcgcgtcgcgggcgactctggcttcgctgcctactctcgctaccggattggcaattacaaattgaacaccgaccactcatctagctccgacaacattgcaggtggcggcggaagtggcggtggaggctctggcggaggaggctcaatgtcagataatggcccacagaaccagcggaacgccccaagaatcacctttggtggtccatctgatagcaccggcagcaaccagaacggggagcgcagcggagcaaggtcaaaacagcgcaggccccagggactgccgaacaacacagcctcatggttcacagccctcacacagcatggtaaagaagatctcaaattcccacgaggtcagggcgtgccaattaataccaatagttctcctgacgatcaaatcggatattacagaagagccacccggcggatccgcggtggtgatgggaaaatgaaggacctcagcccaagatggtatttttattacttggggactggcccagaggcaggactcccttacggggctaataaagacggaattatatgggtggcaaccgaaggagctcttaacacacccaaagatcatattggcaccagaaaccctgccaacaacgcagcaattgtgctgcaactgccgcagggaaccacactgccaaaaggattttatgccgaaggatcacgcggcggatcacaagcctccagtcggtcctccagtcggtcacgcaactccagccgaaattctaccccgggctctagcagggggacttcacccgccagaatggccggcaatggcggagatgcagcactcgcacttttgctgctggataggctgaaccagctggaaagtaaaatgtctggcaagggccagcagcagcaaggacaaaccgtgaccaagaagagcgccgccgaggcctctaagaagccaagacaaaaacgcacagccacgaaggcctataacgtgacacaggcattcggtcgccgcggccctgagcagacgcaaggcaattttggtgatcaggagttgattagacaagggactgactacaagcattggccccaaattgcccagtttgctccttcagcctctgcttttttcgggatgtccagaataggaatggaagtgactcccagtggcacttggcttacatacacaggggctatcaagctggacgataaggacccgaatttcaaggatcaggttatactccttaacaaacatatcgacgcctataagacgtttccacctactgaacctaagaaagacaagaagaagaaggctgatgagactcaagcacttcctcagcgccagaagaaacagcagactgttacactgctgcctgccgccgatctcgacgacttcagcaagcaactgcagcaaagcatgagttcagccgacagtacccaggccggaggaggcggatctgggggcggtgggtcagggggggggggatctgctatcgcgagcgaattttcatctttgccgtcctacgctgcatttgccactgcgcaggaggcctatgagcaggcggtggcgaatggcgactctgaagtggtgcttaaaaagttgaagaaatccctcaacgtagccaaatcagaattcgaccgagatgctgccatgcagcgcaagcttgaaaagatggccgaccaggcaatgacacagatgtacaagcaggcgagatccgaggataaacgagctaaggtgacgtccgcgatgcagacaatgctgttcactatgctgcgcaaactggataacgatgctctgaacaacatcattaataacgccagagatggatgtgttccactgaatatcatacctttgacaactgccgctaagttgatggtagtcatccccgattacaatacttacaaaaacacttgcgacgggaccacgttcacttacgcttccgccctttgggagatccagcaggtcgtggatgccgattcaaagattgtgcaactctccgaaatttcaatggataactcacccaacctcgcgtggcccctgatcgtgaccgcactgcgagctaattccgctgttaaacttcaatga

SEQ ID NO: 31 - protein, 1088 aa, artificial
  Fusion protein including M, N, Ubl1-Nsp3, 3Ecto-Nsp3, Nsp8
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIAGGGGSGGGGSGGGGSMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGSGGGGSGGGGSAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEGGGGSGGGGSGGGGSSNLGMPSYCTGYREGYLNSTNVTIATYCTGSIPCSVCLSGLDSLDTYPSLETIQITISSFKWDLTAFGLVAEWFLAYILFTRGGGGSGGGGSGGGGSAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQ

SEQ ID NO: 32 - DNA, 3267 bp, artificial
  Nucleotide sequence encoding SEQ ID NO31
atggctgatagcaacggcaccatcacagttgaagagctcaagaagcttctggaacagtggaacctggtcattggctttttgtttctgacatggatttgcctgctccagtttgcctacgctaaccggaacagattcctgtacattatcaaactgatcttcctgtggcttctttggcccgtgacccttgcatgcttcgtgctcgccgccgtgtacaggatcaactggataaccggaggaatcgctatcgctatggcttgcctcgttgggttgatgtggctgtcctacttcatcgcttctttccgcctcttcgcacgcacaagatccatgtggtcatttaaccctgaaactaacatcctgcttaatgtgcctctccatggcactatcctcacccgcccactgctggagtcagagctcgtgattggggcggttatcttgcgcggtcatctgaggatagctgggcaccatctggggcggtgtgacataaaggatctgcccaaagagatcacggttgcaacaagtagaactctgagctattacaaactcggagcttcacaaagggtggccggggactccggctttgccgcctattcacggtacagaatcgggaactacaaactcaatacagatcactccagttcctctgataacattgccggcggtgggggcagtggcggcggcgggtcaggcggggggggcagcatgagcgacaacggaccccagaatcagagaaacgctcctcgaatcacatttggtggacctagcgattccactgggagcaatcagaatggtgagaggtccggcgcccgcagcaagcagcggcggccccagggcctgccaaacaacacagctagttggtttaccgctcttacccagcacggaaaggaggatttgaagtttcccaggggacagggggtcccaataaacaccaacagctcacctgatgatcagattggctattaccggagggccacccggcgcatccgcggaggcgatgggaagatgaaagacctgtctccacggtggtacttctattatctgggaactggacccgaggcagggctgccctacggtgcgaataaggatggcattatttgggttgcaacagaaggtgcactgaatacgcctaaggaccacatcggtacaagaaatccagcaaacaacgctgccattgtgcttcaactcccacagggcacgactctgcctaagggcttttacgcagagggaagccgcggtggcagccaagcttccagcagatcctctagtaggtcccggaactcttctcggaactcaacccctggctccagtcgcgggacaagtccagctagaatggccggcaatgggggggatgcagcactcgcactcctgttgctcgacagattgaaccaactggagtctaaaatgtctggtaaaggacagcaacagcagggccagaccgtaacaaagaaatctgccgctgaggcgtctaaaaagccccgccagaagaggaccgccaccaaggcatataatgttactcaggcatttggtagacgcggacctgaacagactcaaggaaacttcggagaccaggagctgatacgccagggaacggactacaagcactggccgcagatagcacagttcgcgccaagcgcaagcgccttttttggcatgtcccgcatcggaatggaagtaacaccttctggaacttggttgacctataccggagccataaaattggacgataaggatcctaacttcaaagaccaagtgatcttgctcaacaaacatattgacgcctataaaacctttcccccgacagagcctaagaaagataaaaaaaagaaggctgacgaaacacaggccctcccacaacggcaaaagaagcagcagactgtcacattgcttcctgccgcagacctggacgacttctccaagcagctgcaacagtctatgtcctccgcagactccacacaagcaggtggtggcggatctggagggggtggctctggtggtggtggcagcgctccaacgaaagtaaccttcggggatgacacagtgatagaggtgcagggatataaatcagtgaacatcacatttgaactggacgagaggatcgataaagtactgaacgaaaaatgctcagcttacaccgtagaactgggcacagaagtcaacgaattcgcatgcgtcgtggctgacgctgttattaaaaccctgcagcctgtcagcgagttgctcacacccctgggaatcgatctggacgaatggagcatggcaacatattacctcttcgacgaatctggagagttcaaactcgcttctcacatgtattgctccttctatcctccggatgaaggcggcggcggatccgggggaggcgggtctggcggcggcggctcttctaatctcgggatgccttcatactgtaccggctacagagagggctacctcaacagcacaaacgtaaccatagccacctattgcaccggaagtataccgtgctccgtgtgtctgagtggacttgatagcctcgacacttatccatcactggaaacaatccaaataaccattagctctttcaaatgggatcttaccgccttcggactcgtggccgagtggtttttggcttatattctgttcaccaggggcggcggtggatctggaggcggtgggagtgggggcggcggctccgctatcgccagtgaatttagcagcctcccatcttatgctgcctttgctaccgctcaggaggcttacgagcaggccgtcgccaacggagatagcgaagtggtgctgaaaaagctgaaaaaatccctgaatgtggccaaaagcgagttcgaccgcgatgctgcaatgcagaggaagctggaaaagatggcagatcaggccatgacgcagatgtacaaacaggctcgctcagaggataaacgggccaaggtgacctctgctatgcagactatgctgtttaccatgctcagaaagctggacaacgatgccctcaataacatcataaataatgcaagagacgggtgcgtgcctcttaacatcatcccccttactacggccgccaaactgatggtcgttataccagattacaatacttacaagaatacgtgcgatgggacaacctttacttatgccagcgctctgtgggagatacagcaggtagtggacgcggatagcaaaattgttcaactgagcgagatctccatggataacagtccgaacctggcctggccactgatcgtcaccgccctcagagctaatagtgccgtcaagctgcaatga

SEQ ID NO: 33 - protein, 15 aa, artificial
  linker
GGGGSGGGGSGGGGS